(19) United States
(12) Patent Application Publication
(10) Pub. No.: US 2023/0136718 A1
(43) Pub. Date: May 4, 2023

(54) FUNCTIONALIZED LIPOSOMES FOR IMAGING MISFOLDED PROTEINS

(71) Applicant: Alzeca Biosciences, LLC, Houston, TX (US)

(72) Inventors: Ananth V. Annapragada, Manvel, TX (US); Peter Nilsson, Linköping (SE); Carlo Medici, Reno, NV (US); Eric A. Tanifum, Houston, TX (US)

(73) Assignee: Alzeca Biosciences, LLC, Houston, TX (US)

(21) Appl. No.: 18/055,100

(22) Filed: Nov. 14, 2022

Related U.S. Application Data
(63) Continuation of application No. 16/751,943, filed on Jan. 24, 2020, now abandoned.
(60) Provisional application No. 62/828,669, filed on Apr. 3, 2019.
(60) Provisional application No. 62/796,189, filed on Jan. 24, 2019.
(60) Provisional application No. 62/796,193, filed on Jan. 24, 2019.
(60) Provisional application No. 62/796,196, filed on Jan. 24, 2019.
(60) Provisional application No. 62/796,198, filed on Jan. 24, 2019.
(60) Provisional application No. 62/796,201, filed on Jan. 24, 2019.

(51) Int. Cl. A61K 49/12; A61K 9/127

(52) U.S. Cl. CPC A61K 49/128; A61K 9/1273

(57) ABSTRACT

Phospholipid-polymer-aromatic conjugates comprising binding ligands, liposome compositions including the phospholipid-polymer-aromatic conjugates, and binding ligands having an affinity for misfolded proteins are described. The phospholipid-polymer-aromatic conjugate may be represented by Structural Formula I: PL-AL-HP-X-BL (I). In Formula I, PL is a phospholipid, AL is an aliphatic linkage, HP is hydrophilic polymer, X is a link between the phospholipid-polymer and the binding ligand, and BL is polycyclic aromatic compound that functions as a binding ligand. The liposomal compositions may be useful for the imaging of misfolded and/or aggregated proteins.

### CROSS-REFERENCE TO RELATED APPLICATIONS

This application claims priority from U.S. Provisional Pat. App. No. 62/828,669, filed on Apr. 3, 2019; U.S. Provisional Pat. App. No. 62/796,186, filed on Jan. 24, 2019; U.S. Provisional Pat. App. No. 62/796,189, filed on Jan. 24, 2019; U.S. Provisional Pat. App. No. 62/796,193, filed on Jan. 24, 2019; U.S. Provisional Pat. App. No. 62/796,196, filed on Jan. 24, 2019; U.S. Provisional Pat. App. No. 62/796,198, filed on Jan. 24, 2019; and U.S. Provisional Pat. App. No. 62/796,201, filed on Jan. 24, 2019, the entire disclosures of which are entirely incorporated by reference herein.

### BACKGROUND

Protein misfolding disorders (PMDs) include, for example, Alzheimer's disease (AD), Parkinson's disease (PD), type 2 diabetes, Huntington's disease (HD), amyotrophic lateral sclerosis (ALS), systemic amyloidosis, prion diseases, and the like. Misfolded and/or aggregated proteins may be formed and accumulate. The misfolded and/or aggregated proteins may induce cellular dysfunction and tissue damage, among other effects. For example, AD may include misfolding and aggregation of beta-amyloid (Aβ), leading to formation of Aβ plaques. Also, PD may include aggregation of αsynuclein (αS) to form fibrils. Both AD and PD may include misfolding and aggregation of tau to form fibrils. Such PMDs may induce cellular dysfunction and tissue damage, among other effects, leading to progressive neurological damage, dementia, and death.

At present, PMDs are typically only conclusively diagnosed by post-mortem histopathological analysis. Diagnosis in living subjects relies primarily on clinical psychiatric testing to detect cognitive impairment. However, the major neuropathological hallmarks of AD—extracellular plaque deposits and intracellular neurofibrillary tangles—manifest long before clinical symptoms are discernable. AP deposits also represent a major risk factor for hemorrhagic stroke.

Various positron emission tomography (PET) imaging agents that bind specifically to amyloid plaques are under investigation or have recently been approved by the FDA, and can be used for the detection of amyloid plaques. However, the spatial resolution of the PET modality is on the order of 5-10 mm, limiting the anatomy-specific information that can be provided by the image. See Moses, W., Nucl Instrum Methods Phys Res A. 648 Supplement 1: S236-S240 (2011). PET imaging also requires the use of radio-isotopes, and all of the attendant radiation-related risks. An amyloid scan is estimated to expose the subject to about 7 mSv of radiation dose, roughly equivalent to several CT scans, as a typical head CT may be about 2 mSv. The short half-life of radioactive PET agents also limits their availability. A non-radioactive amyloid imaging agent would be of significant interest, addressing both the distribution challenges and the radiation dose concerns with current PET imaging agents.

Previous efforts on developing non-radioactive amyloid-targeting MRI agent have primarily focused on either proton T2 (using the T2 relaxivities of iron oxide nanoparticles), or ¹⁹F imaging (using high signal-to-noise ratios achievable due to the absence of endogenous F signal). High T2 relaxivities lead to suppression of the overall signal, making detection and differentiation from inherent hypo-intense regions challenging, and quantitation of the images unreliable. Further, in the case of ¹⁹F imaging, the absence of endogenous MR-visible fluorine means there is no anatomical landmark for the ¹⁹F image.

Other previous work demonstrated that liposomes targeted to amyloid plaque by ligands such as the thioflavine analog Methoxy-XO4, penetrated the blood-brain barrier (BBB), and successfully bound the majority of amyloid plaques in the APP/PSEN1 mouse model of AD. However, existing amyloid binding ligands, including methoxy-XO4, are significantly hydrophobic. In liposomal formulations, this hydrophobicity interferes with the lipid bilayer of the liposome. When loaded with Gd chelates for MRI T1 contrast, methoxy-XO4 targeted liposomes were unstable to the osmotic gradient created by the high Gd chelate internal concentration, and were destabilized. Accordingly, there remains a need for improved imaging agents for detecting misfolded proteins such as those that form amyloid deposits.

### SUMMARY

The present invention provides improved imaging agents. In one aspect, the present invention provides a phospholipid-polymer-aromatic conjugate. The phospholipid-polymer-aromatic conjugate may be represented by Structural Formula II:

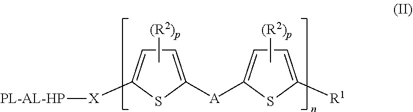

or a pharmaceutically acceptable salt thereof.

Structural Formula II is further defined as follows. PL may be a phospholipid. AL may be an aliphatic linkage. HP may be a hydrophilic polymer. X may be a bond, —O—, —Rⁱ—O—, —Rⁱ—O(C═O)—, —Rⁱ—N(Rⁱⁱ)—O(C═O)—, —Rⁱ—N(Rⁱⁱ)(C═O)—, or —Rⁱ—N(Rⁱⁱ)—. Rⁱ may be a linking group including 1 to 6 carbon atoms. Rⁱⁱ may be hydrogen, C₁-C₆ alkyl, or C₁-C₆ alkoxyalkyl. Each p may be an integer independently selected from 0, 1, or 2, and n may be an integer selected from 1, 2, 3, or 4. Each Rⁱ may be independently selected from H, alkyl, phenyl, and thienyl, wherein Rⁱ other than H may be optionally and independently substituted with 1, 2, or 3 of R⁴. Each A may be independently selected from alkylene, alkenylene, A′-alkylene, A′-alkenylene, alkylene-A′, alkenylene-A′, alkylene-A′-alkylene, alkenylene-A′-alkenylene, and A′. Each A′ may be one of thienylene, phenylene, fluorenylene, benzothienylene, ethylenedioxythienylene, benzothiadiazolylene, and vinylene. Each A may be independently and optionally substituted with 1 or 2 of R³. Each R², R³, and R⁴ may be independently selected from: halogen, hydroxy, alkyl, hydroxyalkyl, aryl, —O-aryl or —(O-alkylene)₁₋₆ optionally substituted with —OH or halogen, amino, aminoalkyl, aminodialkyl, carboxy, sulfonyl, carbamoyl, glycosyl, hydroxyalkoxy, hydroxyalkoxyalkyl, hydroxypolyoxyalkylene, alkoxy, alkoxyalkyl, polyoxyalkylene, carboxy, carboxyalkyl, carboxyalkoxy, carboxyalkoxyalkyl, carboxypolyoxyalkylene, alkoxycarbonyl, alkoxycarbonylalkyl, alkoxycarbonylalkoxy, alkoxycarbonylalkoxyalkyl, alkoxycarbonylpolyoxyalkylene, amino, aminoalkyl, aminodialkyl, alkylaminoalkyl, dialkylaminoalkyl, aminoalkoxy, alkylaminoalkoxy, dialkylaminoalkoxy, aminopolyoxyalkylene, alkylaminopolyoxyalkylene, dialkylaminopolyoxyalkylene, aminoalkoxyalkyl, alkylaminoalkoxyalkyl, dialkylaminoalkoxyalkyl, (amino) (carboxy)alkyl, (alkylamino) (carboxy)alkyl, (dialkylamino) (carboxy)alkyl, (amino) (carboxy)alkoxy, (alkylamino) (carboxy)alkoxy, (dialkylamino) (carboxy)alkoxy, (amino) (carboxy)alkoxyalkyl, (alkylamino) (carboxy) alkoxyalkyl, (dialkylamino) (carboxy) alkoxyalkyl, (amino) (carboxy) polyoxyalkylene, (alkylamino) (carboxy) polyoxyalkylene, (dialkylamino) (carboxy) polyoxyalkylene, (alkoxycarbonyl) (amino) alkyl, (alkoxycarbonyl) (alkylamino) alkyl, (alkoxycarbonyl) (dialkylamino) alkyl, (alkoxycarbonyl) (amino) alkoxy, (alkoxycarbonyl) (alkylamino) alkoxy, (alkoxycarbonyl) (dialkylamino) alkoxy, (alkoxycarbonyl) (amino) alkoxyalkyl, (alkoxycarbonyl) (alkylamino) alkoxyalkyl, (alkoxycarbonyl) (dialkylamino) alkoxyalkyl, (alkoxycarbonyl) (amino) polyoxyalkylene, (alkoxycarbonyl) (alkylamino) polyoxyalkylene, (alkoxycarbonyl) (dialkylamino) polyoxyalkylene, acylamino, acylaminoalkyl, acylaminoalkoxy, acylaminoalkoxyalkyl, acylaminopolyoxyalkylene, acylalkylamino, acylalkylaminoalkyl, acylalkylaminoalkoxy, acylalkylaminoalkoxyalkyl, acylalkylaminopolyoxyalkylene, hydrazinocarbonyl, hydrazinocarbonylalkyl, hydrazinocarbonylalkoxy, hydrazinocarbonylalkoxyalkyl, hydrazinocarbonylpolyoxyalkylene, nitro, nitroalkyl, nitroalkoxy, nitroalkoxyalkyl, nitropolyoxyalkylene, cyano, cyanoalkyl, cyanoalkoxy, cyanoalkoxyalkyl, cyanopolyoxyalkylene, sulfo, sulfoalkyl, sulfoalkoxy, sulfoalkoxyalkyl and sulfopolyoxyalkylene. Two R², attached to the same thiophene ring, may together represent alkylenedioxy, optionally substituted with sulfoalkyl, sulfoalkoxy, sulfoalkoxyalkyl or sulfopolyoxyalkylene. Each alkyl or alkylene group represented in Structural Formula II or variables therein may be independently selected from C₁-C₆ alkyl or C₁-C₆ alkylene. Each alkenyl or alkenylene group represented in Structural Formula II or variables therein may be independently selected from C₂-C₆ alkenyl or C₂-C₆ alkenylene. Each NH₂ represented in Structural Formula II or variables therein may optionally and independently be protected by a group selected from tert-butyl carbamate, benzyl carbamate or 9-fluorenylmethyl carbamate or substituted with biotinyl.

In another aspect, a liposomal composition is provided. The liposomal composition may include a membrane. The membrane may include a phospholipid-polymer-aromatic conjugate represented by Structural Formula II:

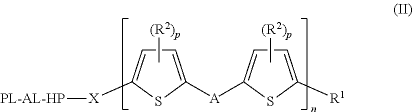

or a pharmaceutically acceptable salt thereof. In Structural Formula II, the variables R¹, R², p, n, A, X, HP, AL, and PL may be independently selected from the corresponding values described for Structural Formula II herein.

Another aspect of the invention a method for imaging one or more misfolded and/or aggregated proteins in a subject. The method may include introducing into the subject a detectable quantity of a liposomal composition. The method may include allowing sufficient time for the liposomal composition to be associated with the one or more misfolded and/or aggregated proteins. The method may include detecting the liposomal composition associated with the one or more misfolded and/or aggregated proteins. The liposomal composition of the method may include a membrane. The membrane may include a phospholipid-polymer-aromatic conjugate represented by Structural Formula II:

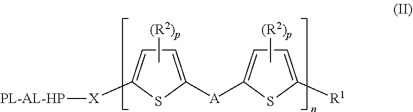

or a pharmaceutically acceptable salt thereof. In Structural Formula II, the variables R¹, R², p, n, A, X, HP, AL, and PL may be independently selected from the corresponding values described herein.

In another embodiment, a liposomal composition is provided for use in a method for imaging one or more misfolded and/or aggregated proteins in a subject. The method may include introducing into the subject a detectable quantity of a liposomal composition. The method may include allowing sufficient time for the liposomal composition to be associated with the one or more misfolded and/or aggregated proteins. The method may include detecting the liposomal composition associated with the one or more misfolded and/or aggregated proteins. The liposomal composition of the method may include a membrane. The membrane may include a phospholipid-polymer-aromatic conjugate represented by Structural Formula II:

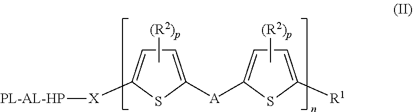

or a pharmaceutically acceptable salt thereof. In Structural Formula II, the variables R¹, R², p, n, A, X, HP, AL, and PL may be independently selected from the corresponding values described herein.

In another aspect, a binding ligand represented by Structural Formula III is provided:

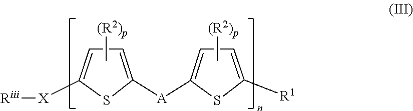

or a pharmaceutically acceptable salt thereof. In Structural Formula III, Rⁱⁱⁱ may be hydrogen, hydroxyl, H—Rⁱ—, HO—R¹—, H—R¹—N(Rⁱⁱ)—, or HO—Rⁱ—N(Rⁱⁱ). R¹ may be a linking group including 1 to 6 carbon atoms, e.g., one of: alkylene and alkoxyalkylene. R¹ may be substituted with zero, one or more of: hydroxyl, C₁-C₆ alkyl, and C₁-C₆ hydroxyalkyl. Rⁱⁱ may be hydrogen, C₁-C₆ alkyl, or C₁-C₆ alkoxyalkyl. Rⁱⁱ other than hydrogen may be independently substituted with zero, one or more of: halogen; —OH; alkyl, —O-alkyl, aryl, —O-aryl or —(O-alkylene)₁₋₆ optionally substituted with —OH or halogen; —NH₂; —NH-alkyl; —N-dialkyl; carboxyl; sulfonyl; carbamoyl; and glycosyl. In Structural Formula III, the variables R¹, R², p, n, A, and X may be independently selected from the corresponding values described for Structural Formula II herein.

In one embodiment, a phospholipid-polymer-aromatic conjugate is provided for use in a method for imaging one or more misfolded and/or aggregated proteins in a subject. The method may include introducing into the subject a detectable quantity of a liposomal composition. The method may include allowing sufficient time for the liposomal composition to be associated with the one or more misfolded and/or aggregated proteins. The method may include detecting the liposomal composition associated with the one or more misfolded and/or aggregated proteins. The membrane may include the phospholipid-polymer-aromatic conjugate. The aromatic moiety in the phospholipid-polymer-aromatic conjugate may be represented by Structural Formula II:

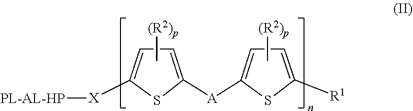

or a pharmaceutically acceptable salt thereof. In Structural Formula II, the variables R¹, R², p, n, A, X, HP, AL, and PL may be independently selected from the corresponding values described for Structural Formula II herein.

The present methods, compound, conjugates, and liposomes are believed to readily facilitate crossing the BBB in humans. It is known from MRI studies performed in AD and MCI patients that the BBB may indeed be compromised and the extent of compromise may be independent of amyloid burden. Also, a recent study using DCE-MRI confirmed that the BBB in the aging human hippocampus breaks down and becomes permeable. Accordingly, the present methods, ligands, conjugates and liposomes may function in humans.

The described MRI imaging may offer a number of substantial benefits over current non-invasive imaging technologies, such as PET imaging, including increased availability, reduced cost, and enhanced resolution. The availability of known, approved PET agents for the imaging of amyloid plaques may be limited and restricted to large academic medical centers. By contrast, the work described herein may offer worldwide availability. Moreover, T1 agents may be extremely attractive because of their positive signal, leading to increased confidence in signal interpretation. The work described herein is targeted for use in low field (1-3T) scanners consistent with state-of-the-art MRI scanners for human imaging.

In another aspect, a kit for imaging one or more misfolded and/or aggregated proteins in a subject is provided. The kit may include instructions and a liposomal composition. The instructions may direct a user to introduce into the subject a detectable quantity of the liposomal composition. The instructions may direct the user to allow sufficient time for the liposomal composition to be associated with the one or more misfolded and/or aggregated proteins. The instructions may direct the user to detect the liposomal composition associated with the one or more misfolded and/or aggregated proteins. The liposomal composition of the kit may include a membrane. The membrane may include a phospholipid-polymer-aromatic conjugate represented by Structural Formula II:

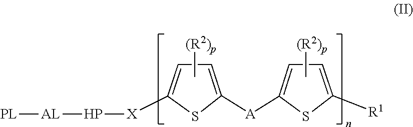

or a pharmaceutically acceptable salt thereof. In Structural Formula II, the variables R¹, R², p, n, A, X, HP, AL, and PL may be independently selected from the corresponding values described for Structural Formula II herein.

In another embodiment, a kit for imaging one or more misfolded and/or aggregated proteins in a subject is provided. The kit may include the phospholipid-polymer-aromatic conjugate represented by Structural Formula II:

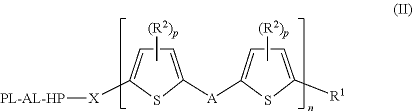

or a pharmaceutically acceptable salt thereof. In Structural Formula II, the variables R¹, R², p, n, A, X, HP, AL, and PL may be independently selected from the corresponding values described for Structural Formula II herein. The kit may include instructions directing the user to employ the phospholipid-polymer-aromatic conjugate represented by Structural Formula II to form the liposomal composition. The instructions may direct a user to introduce into the subject a detectable quantity of the liposomal composition. The instructions may direct the user to allow sufficient time for the liposomal composition to be associated with the one or more misfolded and/or aggregated proteins. The instructions may direct the user to detect the liposomal composition associated with the one or more misfolded and/or aggregated proteins.

### BRIEF DESCRIPTION OF THE DRAWINGS

In the accompanying figures, chemical formulas, chemical structures, and experimental data are given that, together with the detailed description provided below, describe example embodiments of the claimed invention.

FIG. 1A provides chemical drawings showing Structural Formulas i-viii.

FIG. 1B provides chemical drawings showing Structural Formulas ix-xiv.

FIG. 2A provides chemical drawings showing Structural Formulas xv-xiii.

FIG. 2B provides chemical drawings showing Structural Formulas xix-xxii.

FIG. 2C provides chemical drawings showing Structural Formulas xxiii-xxvi.

FIG. 3 is a chemical drawing depicting structures for Conjugate A and Conjugate A′.

FIG. 4A is a reaction scheme illustrating the chemical reactions described in Example 1.

FIG. 4B is a mass spectrum showing that the found average neutral mass for Conjugate-A was 5141.23, calculated for molecular weight 5142.21 (C₂₃₇H₄₃₁N₅O₁₀₀PS₅).

FIG. 5A is a graph showing a standard curves for free ligand p-FTAA and Conjugate-A-liposomes were quantified (>43% in the supernatant) used for binding curve assay, using a standard curve.

FIG. 5B is a graph showing experimental data and calculated fit lines for Conjugate-A-liposomes and the free ligand, p-FTAA for Aβ binding. The binding constant (k_{b}) for Conjugate-A-liposomes was 2.0 nM, half of that for the free ligand, p-FTAA, which was 4 nM.

FIG. 6A is a photograph showing that Conjugate-A-liposomes readily entered deep into brain tissues to stain concentrated Aβ deposits.

FIG. 6B is a photograph showing that Conjugate-A-liposomes readily entered deep into brain tissues to stain tau tangles.

FIG. 6C is a photograph showing that Conjugate-A-liposomes readily entered deep into brain tissues to stain neuritic plaques.

FIG. 6D is a photograph showing that Conjugate-A-liposomes readily entered deep into brain tissues to stain diffuse plaques.

FIG. 7A is a graph showing experimental data and calculated fit lines for Conjugate-A-liposomes for α-Synuclein, from which a dissociation constant, K_{d} of 1.75 nM was determined for Conjugate A liposomes.

FIG. 7B is a graph showing experimental data and calculated fit lines for the free ligand p-FTAA for α-Synuclein, from which a dissociation constant, K_{d} of 3 nM was determined for free ligand p-FTAA.

FIG. 8 provides the image of a SDS PAGE gel run to confirm the phosphorylation of tau.

FIG. 9A is a graph showing an increase in fluorescence for p-FTAA with tau fibrils in comparison to p-FTAA only fluorescence, indicating binding of pFTAA to tau fibrils.

FIG. 9B is a graph of the ratio of the fluorescence of tau fibril—pFTAA to pFTAA only.

### DETAILED DESCRIPTION

Provided are phospholipid-polymer-aromatic conjugates comprising binding ligands, liposome compositions including the phospholipid-polymer-aromatic conjugates, and binding ligands having an affinity for misfolded proteins are described. The liposomal compositions may be useful for the imaging of misfolded and/or aggregated proteins.

### Phospholipid-Polymer-Aromatic Conjugates

In one aspect, a phospholipid-polymer-aromatic conjugate is provided. The phospholipid-polymer-aromatic conjugate may be represented by Structural Formula I:

PL-AL-HP-X-BL (I)

or a pharmaceutically acceptable salt thereof. PL is a phospholipid. AL is an aliphatic linkage. HP is a hydrophilic polymer. X is a link between the phospholipid-polymer and the binding ligand, which can be simply a bond, and BL is a binding ligand that is an polycyclic aromatic compound, and in particular polycyclic aromatic compounds having an affinity for one or more misfolded proteins.

In some embodiments, the phospholipid-polymer aromatic conjugate has a structure according to Structural Formula II

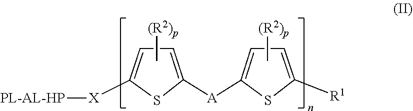

or a pharmaceutically acceptable salt thereof, is provided. X may be a bond, —O—, -Ri O—, -Ri O(C═O), Ri-N(Rⁱⁱ) O(C═O), Ri-N(Rii)(C═O)—, or Ri-N(Rii)—. Ri may be a linking group including 1 to 6 carbon atoms, e.g., one of: alkylene and alkoxyalkylene. Rii may be hydrogen, C₁-C₆ alkyl, or C₁-C₆ alkoxyalkyl.

In Structural Formula II, n may be independently selected in a range of between about 1 and about 12, between 1 and about 8, between 1 and about 4, or, an integer, for example selected from 1, 2, 3, or 4. Each p may be an integer independently selected from 0, 1, or 2. Each R¹ may be independently selected from H, alkyl, phenyl, and thienyl, wherein R¹ other than H may be optionally and independently substituted with 1, 2, or 3 of R⁴. Each A may be independently selected from alkylene, alkenylene, A′-alkylene, A′-alkenylene, alkylene-A′, alkenylene-A′, alkylene-A′-alkylene, alkenylene-A′-alkenylene, and A′. Each A′ may be one of thienylene, phenylene, fluorenylene, benzothienylene, ethylenedioxythienylene, benzothiadiazolylene, and vinylene.

Each A may be independently and optionally substituted with 1 or 2 of R³. Each R², R³, and R⁴ may be independently selected from: halogen, hydroxy, alkyl, hydroxyalkyl, aryl, —O-aryl or —(O-alkylene)₁₋₆ optionally substituted with —OH or halogen, amino, aminoalkyl, aminodialkyl, carboxy, sulfonyl, carbamoyl, glycosyl, hydroxyalkoxy, hydroxyalkoxyalkyl, hydroxypolyoxyalkylene, alkoxy, alkoxyalkyl, polyoxyalkylene, carboxy, carboxyalkyl, carboxyalkoxy, carboxyalkoxyalkyl, carboxypolyoxyalkylene, alkoxycarbonyl, alkoxycarbonylalkyl, alkoxycarbonylalkoxy, alkoxycarbonylalkoxyalkyl, alkoxycarbonylpolyoxyalkylene, amino, aminoalkyl, aminodialkyl, alkylaminoalkyl, dialkylaminoalkyl, aminoalkoxy, alkylaminoalkoxy, dialkylaminoalkoxy, aminopolyoxyalkylene, alkylaminopolyoxyalkylene, dialkylaminopolyoxyalkylene, aminoalkoxyalkyl, alkylaminoalkoxyalkyl, dialkylaminoalkoxyalkyl, (amino) (carboxy)alkyl, (alkylamino) (carboxy)alkyl, (dialkylamino) (carboxy)alkyl, (amino) (carboxy)alkoxy, (alkylamino) (carboxy)alkoxy, (dialkylamino) (carboxy)alkoxy, (amino) (carboxy)alkoxyalkyl, (alkylamino) (carboxy) alkoxyalkyl, (dialkylamino) (carboxy) alkoxyalkyl, (amino) (carboxy) polyoxyalkylene, (alkylamino) (carboxy) polyoxyalkylene, (dialkylamino) (carboxy) polyoxyalkylene, (alkoxycarbonyl) (amino) alkyl, (alkoxycarbonyl) (alkylamino) alkyl, (alkoxycarbonyl) (dialkylamino) alkyl, (alkoxycarbonyl) (amino) alkoxy, (alkoxycarbonyl) (alkylamino) alkoxy, (alkoxycarbonyl) (dialkylamino) alkoxy, (alkoxycarbonyl) (amino) alkoxyalkyl, (alkoxycarbonyl) (alkylamino) alkoxyalkyl, (alkoxycarbonyl) (dialkylamino) alkoxyalkyl, (alkoxycarbonyl) (amino) polyoxyalkylene, (alkoxycarbonyl) (alkylamino) polyoxyalkylene, (alkoxycarbonyl) (dialkylamino) polyoxyalkylene, acylamino, acylaminoalkyl, acylaminoalkoxy, acylaminoalkoxyalkyl, acylaminopolyoxyalkylene, acylalkylamino, acylalkylaminoalkyl, acylalkylaminoalkoxy, acylalkylaminoalkoxyalkyl, acylalkylaminopolyoxyalkylene, hydrazinocarbonyl, hydrazinocarbonylalkyl, hydrazinocarbonylalkoxy, hydrazinocarbonylalkoxyalkyl, hydrazinocarbonylpolyoxyalkylene, nitro, nitroalkyl, nitroalkoxy, nitroalkoxyalkyl, nitropolyoxyalkylene, cyano, cyanoalkyl, cyanoalkoxy, cyanoalkoxyalkyl, cyanopolyoxyalkylene, sulfo, sulfoalkyl, sulfoalkoxy, sulfoalkoxyalkyl and sulfopolyoxyalkylene. Two R², attached to the same thiophene ring, may together represent alkylenedioxy, optionally substituted with sulfoalkyl, sulfoalkoxy, sulfoalkoxyalkyl or sulfopolyoxyalkylene.

Each alkyl or alkylene group represented in Structural Formula II or variables therein may be independently selected from C₁-C₆ alkyl or C₁-C₆ alkylene. Each alkenyl or alkenylene group represented in Structural Formula II or variables therein may be independently selected from C₂-C₆ alkenyl or C₂-C₆ alkenylene. Each NH₂ represented in Structural Formula II or variables therein may optionally and independently be protected by a group selected from tert-butyl carbamate, benzyl carbamate or 9-fluorenylmethyl carbamate or substituted with biotinyl.

In various embodiments, each amine and heteroaromatic ring nitrogen may independently and optionally be alkylated to form a quaternary ammonium accompanied by a pharmaceutically acceptable anion, e.g., a halide ion, an acetate ion, and the like. For example, each amine, thiazole, and benzothiazole nitrogen may independently and optionally be alkylated to form a quaternary ammonium accompanied by a pharmaceutically acceptable anion, e.g., a halide ion, an acetate ion, and the like.

In several embodiments, a binding ligand corresponding to the aromatic compound represented by Structural Formula III is provided:

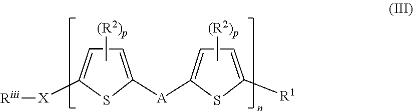

or a pharmaceutically acceptable salt thereof. In Structural Formula III, Rⁱⁱⁱ may be hydrogen, hydroxyl, H—Rⁱ—, HO—Rⁱ—, H—Rⁱ—N(R′)—, or HO—Rⁱ—N(Rⁱⁱ). In some embodiments, Rⁱⁱⁱ may be hydroxyl, H—Rⁱ—, HO—Rⁱ—, H—Rⁱ—N(Rⁱⁱ)—, or HO—Rⁱ—N(Rⁱⁱ)—. Rⁱⁱ may be H—Rⁱ—, HO—Rⁱ—, H—Rⁱ—N(Rⁱⁱ)—, or HO—Rⁱ—N(R′)—. Rⁱⁱⁱ may be H—Rⁱ— or H—R¹—N(Rⁱⁱ)—. Rⁱⁱⁱ may be HO—Rⁱ— or HO—Rⁱ—N(Rⁱⁱ)—. R^{H} may be H—Rⁱ— or HO—Rⁱ—. Rⁱ may be a linking group including 1 to 6 carbon atoms, e.g., one of: alkylene and alkoxyalkylene. Rⁱ may be substituted with zero, one or more of: hydroxyl, C₁-C₆ alkyl, and C₁-C₆ hydroxyalkyl. Rⁱⁱ may be hydrogen, C₁-C₆ alkyl, or C₁-C₆ alkoxyalkyl. Rⁱ other than hydrogen may be independently substituted with zero, one or more of: halogen; —OH; alkyl, —O-alkyl, aryl, —O-aryl or —(O-alkylene)₁₋₆ optionally substituted with —OH or halogen; —NH₂; —NH-alkyl; —N-dialkyl; carboxyl; sulfonyl; carbamoyl; and glycosyl. In Structural Formula III, the variables R¹, R², p, n, A, and X may be independently selected from the corresponding values described for Structural Formula II herein.

In various embodiments, conjugates or binding ligands may be uniform or non-uniform with respect to structural repeat units in corresponding chemical structures depicted herein. For example, Structural Formulas II and III each include a bracketed repeat unit, denoted by the repeat unit variable n. Other structures disclosed herein disclose repeat units, such as the bracketed ethylene oxide repeat units shown in various structures and denoted by the repeat unit variables q and r. Some structures disclosed herein depict —CH₂— repeat units denoted by the repeat unit variable s. For each structure which includes repeat unit variables n, q, r, and/or s, one of ordinary skill in the art will appreciate that the variables have integer values for a particular molecule. One of ordinary skill in the art will also appreciate that for a non-uniform collection of molecules described by a structure with repeat unit variables n, q, r, and/or s, each variable may independently be an average value over the non-uniform collection of molecules, and may have average values represented by fractional values between integers. One of ordinary skill in the art will also recognize that a uniform collection of molecules may be described with repeat unit variables n, q, r, and/or s that are, or are substantially integer values.

Accordingly, in some embodiments, the conjugates or binding ligands represented by Structural Formulas II or III may be uniform with respect to one or more of n, q, r, and/or s. The conjugates or binding ligands represented by Structural Formulas II or III may be substantially uniform with respect to one or more of n, q, r, and/or s. In some embodiments, the conjugates or binding ligands represented by Structural Formulas II or III may include a mixture of at least two uniform conjugates or binding ligands.

In some embodiments, n may be from 1 to 4; e.g., from 1 to 3, such as 1 or 2; and each p may be independently 0-2; e.g. 0 or 1; each A may be a moiety independently selected from thienylene, phenylene, fluorenylene, benzothienylene, ethylenedioxythienylene, benzothiadiazolylene and vinylene; e.g., thienylene, phenylene, and ethylenedioxythienylene; or e.g., thienylene. Each A may be optionally substituted with 1 or 2 groups R³ as described herein. Each R¹ may be independently selected from H, phenyl and thienyl, e.g., H and thienyl. Each R¹ may be optionally substituted with 1-3 groups R⁴; e.g. 1 or 2 groups, or 1 group R⁴, as described herein. In some embodiments, each A may be unsubstituted.

In some embodiments, thienylene, ethylenedioxythienylene, or benzothienylene when present in A may be, for example, coupled 2,5 with respect to the thienyl ring:

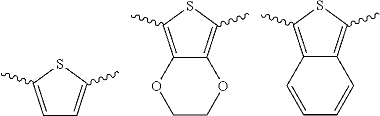

In several embodiments, phenylene, when present in A, may be coupled 1,4, or para:

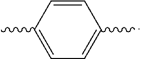

Benzothiadiazolylene, when present in A, may be 4,7-coupled:

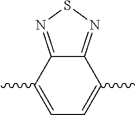

Fluorenyl, when present in A, may be 2,7 coupled:

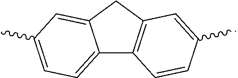

Vinylene, when present in A, may be in a cis or trans configuration, e.g., the thiophene rings coupled through a vinylene may be in a trans configuration:

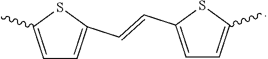

In several embodiments of Structural Formulas II and III, R², R³, and R⁴ may be independently substituted with zero, one or more of: F, C₁, Br, I, alkyl, aryl, —OH, —O-alkyl, —O-aryl, —NH₂, —NH-alkyl, —N-dialkyl, carboxyl, sulfonyl, carbamoyl, and glycosyl.

In various embodiments of Structural Formulas II and III, X may be a bond. X may be —O— or —R¹—O—. X may be —R—O(C═O)—, —R¹—N(Rⁱⁱ)—O(C═O), or —R¹—N(Rⁱⁱ)(C═O)—. X may be —R¹—N(Rⁱⁱ)—. R¹ may be substituted with zero, one or more —OH. Rⁱⁱ may be C₁-C₆ alkyl substituted with zero, one or more of: —OH and alkyl optionally substituted with one or more —OH. Rⁱⁱ may be C₁-C₃ alkyl or hydroxyalkyl.

In some embodiments, each R², R³ and R⁴ may be independently selected from halogen, alkoxy, alkoxyalkyl, polyoxyalkylene, carboxy, carboxyalkyl, carboxyalkoxy, carboxy polyoxyalkylene, alkoxycarbonyl, alkoxycarbonylalkyl, alkoxycarbonylalkoxy, alkoxycarbonyl polyoxyalkylene, amino, alkylamino, dialkylamino, aminoalkyl, alkylaminoalkyl, dialkylaminoalkyl, aminoalkoxy, alkylaminoalkoxy, dialkylaminoalkoxy, amino polyoxyalkylene, alkylamino polyoxyalkylene, dialkylamino polyoxyalkylene, (amino) (carboxy) alkyl, (alkylamino) (carboxy)alkyl, (dialkylamino) (carboxy) alkyl, (amino) (carboxy)alkoxy, (alkylamino) (carboxy) alkoxy, (dialkylamino) (carboxy) alkoxy, (amino) (carboxy) polyoxyalkylene, (alkylamino) (carboxy) polyoxyalkylene, (dialkylamino) (carboxy) polyoxyalkylene, (alkoxycarbonyl) (amino)alkyl, (alkoxycarbonyl) (alkylamino)alkyl, (alkoxycarbonyl) (dialkylamino) alkyl, (alkoxycarbonyl) (amino) alkoxy, (alkoxycarbonyl) (alkylamino) alkoxy, (alkoxycarbonyl) (dialkylamino) alkoxy, (alkoxycarbonyl) (amino)polyoxyalkylene, (alkoxycarbonyl) (alkylamino) polyoxyalkylene, (alkoxycarbonyl) (dialkylamino) polyoxyalkylene, (alkoxycarbonyl) (alkylamino) alkoxy, (alkoxycarbonyl) (dialkylamino) alkoxy, and sulfoalkyl, sulfoalkoxyalkyl, and sulfopolyoxyalkylene. Two R² attached to the same ring, e.g., thienyl, may be taken together to represent alkylenedioxy, optionally substituted with sulfoalkyl, sulfoalkoxyalkyl or sulfopolyoxyalkylene. Each NH² may optionally be protected as a tert-butyl carbamate, benzyl carbamate or 9-fluorenylmethyl carbamate or substituted with a biotinyl moiety.

In some embodiments, each R², R³ and R⁴ may be independently selected from halogen, alkoxy, carboxy, carboxyalkyl, alkoxycarbonylalkyl, aminoalkyl, diaminoalkoxy, (amino) (carboxy)alkoxyalkyl, (alkylamino) (carboxy)alkoxyalkyl, (dialkylamino) (carboxy)alkoxyalkyl, (alkoxycarbonyl) (amino) alkoxyalkyl, (alkoxycarbonyl) (alkylamino) alkoxyalkyl, (alkoxycarbonyl) (dialkylamino) alkoxyalkyl, and sulfoalkoxyalkyl. Two R² attached to the same ring, e.g., thienyl ring, may be taken together to represent alkylene dioxy, optionally substituted with sulfoalkyl, sulfoalkoxy, sulfoalkoxyalkyl or sulfopolyoxyalkylene. Each primary amino group may be optionally protected as a tert-butyl carbamate, benzyl carbamate or 9-fluorenylmethyl carbamate.

In some embodiments, the phospholipid-polymer-aromatic conjugate of Structural Formula II may be represented by one of:

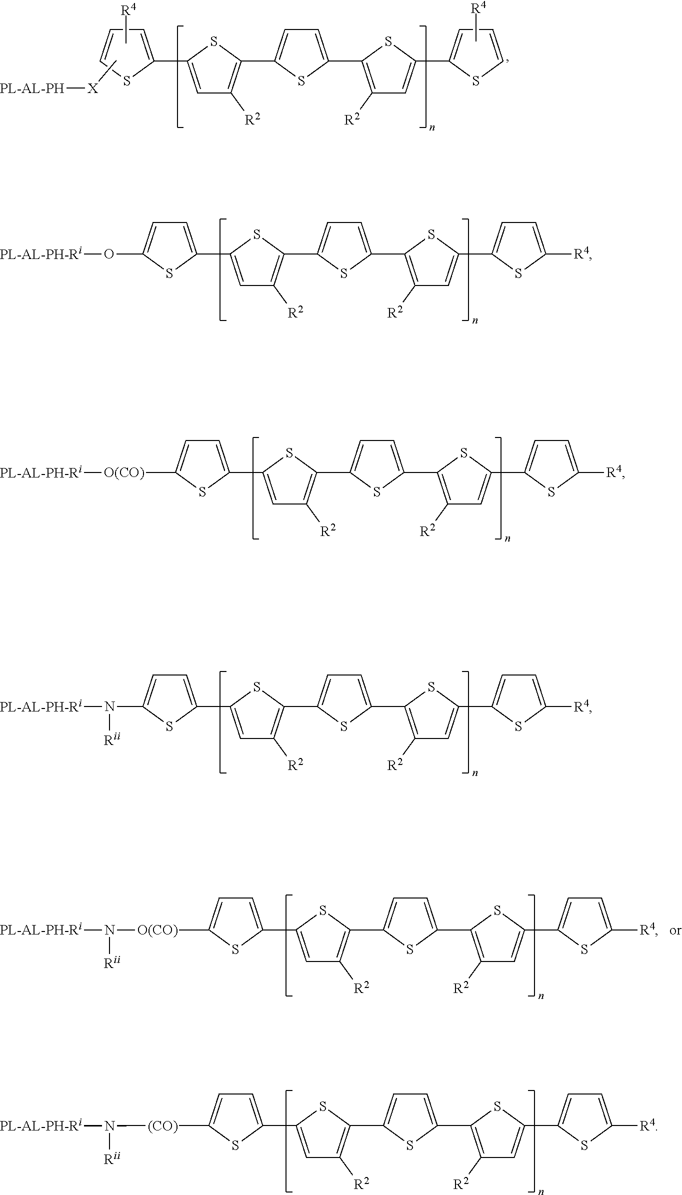

Likewise, a binding ligand of Structural Formula III may be represented by:

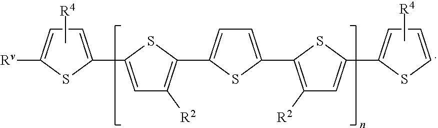

The variable m may be 1-4; e.g. 1-3, or 1 or 2; e.g. 1. Each R² may be independently selected from carboxy, carboxyalkyl, alkoxycarbonylalkyl, aminoalkyl, (amino) (carboxy)alkoxyalkyl, (dialkylamino) (carboxy) alkoxyalkyl, (amino) (alkoxycarbonyl)alkoxyalkyl and (amino) (phenoxycarbonyl) alkoxyalkyl. Each R⁴ may be independently selected from hydrogen, halogen, carboxy, carboxyalkyl, alkoxycarbonylalkyl, aminoalkyl, (amino) (carboxy) alkoxyalkyl, (dialkylamino) (carboxy) alkoxyalkyl, (amino) (alkoxycarbonyl)alkoxyalkyl, (amino) (phenoxycarbonyl)alkoxyalkyl, acylamino, acylaminoalkyl, acylalkylamino and acylalkylaminoalkyl.

In some embodiments, each R² may be independently selected from carboxy, carboxymethyl, methoxycarbonylmethyl, aminomethyl, (amino) (carboxy)ethoxyethyl, (dimethylamino) (carboxy) ethoxyethyl, (amino) (methoxycarbonyl) ethoxyethyl and (amino) (phenoxycarbonyl)ethoxyethyl. Each R⁴ may be independently selected from hydrogen, halogen, carboxy, carboxymethyl, methoxycarbonylmethyl, aminomethyl, (amino) (carboxy)ethoxyethyl, (dimethylamino) (carboxy)ethoxyethyl, (amino) (methoxycarbonyl) ethoxyethyl, and (amino) (phenoxycarbonyl)ethoxyethyl.

In some embodiments, all groups R² may be the same, or all R² and R⁴ groups may be the same. For example, all R² groups, or all R² and R⁴ groups, may be the same one of: —(C═O)OH or a metal salt thereof, e.g., —(C═O)O.M⁺, where M⁺ is a metal ion, e.g., an alkali metal ion such as sodium ion; —(C═O)—C₁-C₆ alkyl, e.g., —(C═O)OCH₃; —CH₂(C═O)OH or a metal salt thereof, e.g., —(C═O)O.M⁺, where M⁺ is a metal ion, e.g., an alkali metal ion such as sodium ion; —CH₂(C═O)—C₁-C₆ alkyl, e.g., —CH₂(C═O)OCH₃; —NH₂; —CH₂NH₂; —CH₂(CH)(NH₂)((C═O)OH); or —OCH₂(CH)(NH₂)((C═O)OH). Each R² or R⁴ that is —CH₂(CH)(NH₂)((C═O)OH) may independently be R or S, or may be the same of R and S. Each R² or R⁴ that is —OCH₂(CH)(NH₂)((C═O)OH) may independently be R or S, or may be the same of R and S.

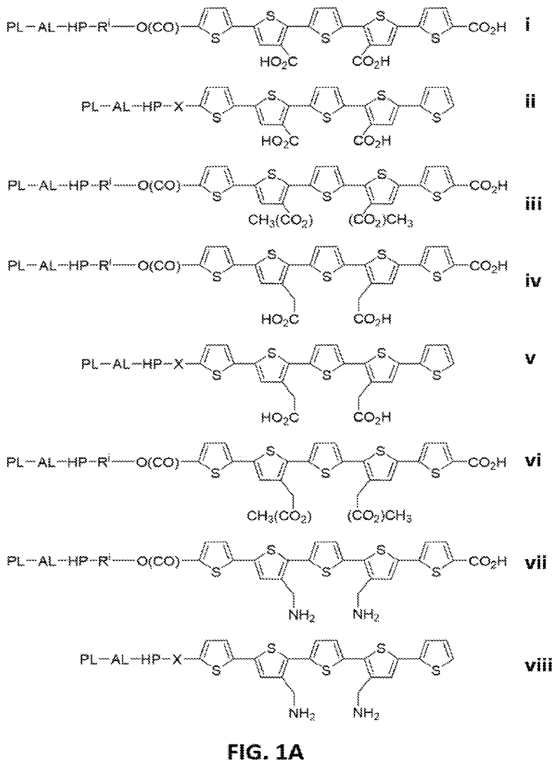

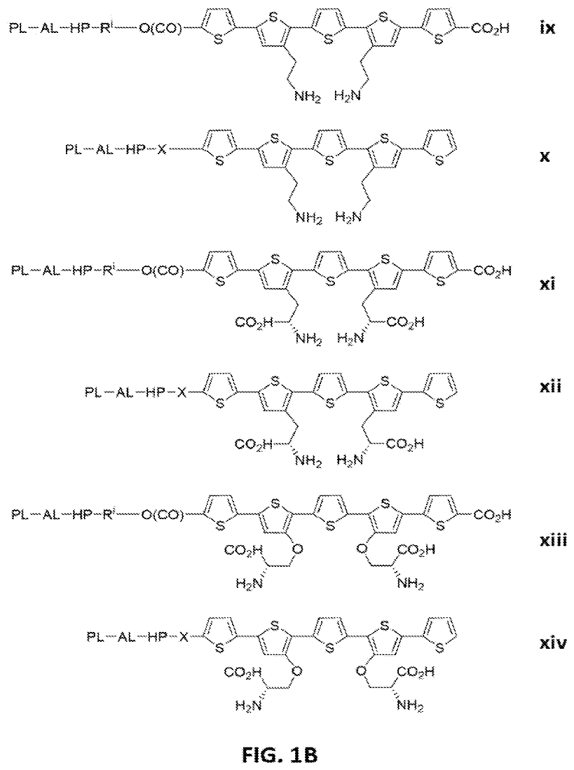

In various embodiments, the phospholipid-polymer-aromatic conjugate is represented by one of Structural Formulas i-xiv shown in FIGS. 1A and 1B. Each variable therein, e.g., PL, AL, HP, X, R, and Rⁱⁱ, may be as described herein.

In some embodiments, the phospholipid-polymer-aromatic conjugate of Structural Formula II may be represented by:

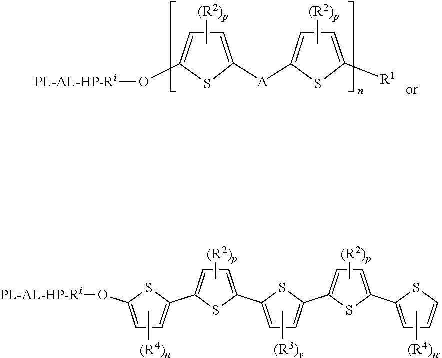

The variable p may be any integer independently selected from 0, 1, and 2; v may be any integer independently selected from 0, 1, and 2; and u may be any integer independently selected from 0, 1, 2, and 3; provided that not all of p, v, and u are simultaneously 0.

Similarly, the binding ligands of Structural Formula III may be represented by:

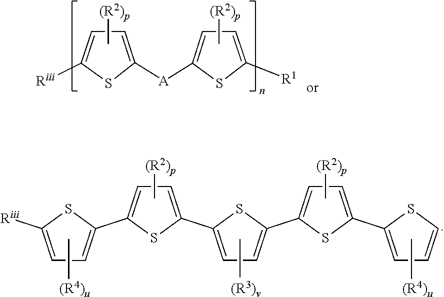

Rⁱⁱⁱ may be H, hydroxyl, H—Rⁱ—, HO—R¹—, H—R¹—N(Rⁱⁱ)—, or HO—R¹—N(Rⁱⁱ)—. The variable p may be any integer independently selected from 0, 1, and 2; v may be any integer independently selected from 0, 1, and 2; and u may be any integer independently selected from 0, 1, 2, and 3; provided that not all of p, v, and u are simultaneously 0. In some embodiments, Rⁱⁱ may be hydroxyl.

In several embodiments, the phospholipid-polymer-aromatic conjugate of Structural Formula II may be represented by:

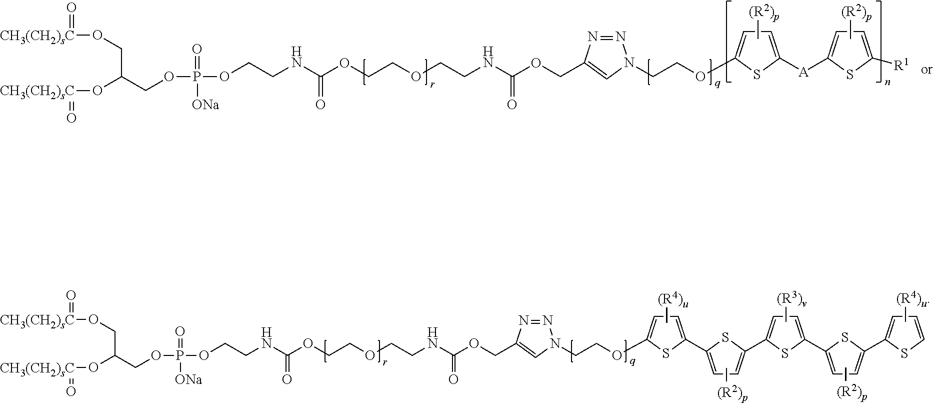

The variable r may be independently selected from a range of between about 10 to about 100, between about 60 to about 100, between about 70 to about 90, between about 75 to about 85, about 77, and the like. The variable s may be independently selected from a range of between about 12 and about 18, one of: 12, 13, 14, 15, 16, 17, or 18, one of 12, 14, 16, or 18, or 14 or 16. For example, r may be 77 and s may be 14. In another example, r may be 77 and s may be 16. The variable p may be any integer independently selected from 0, 1, and 2; v may be any integer independently selected from 0, 1, and 2; and u may be any integer independently selected from 0, 1, 2, and 3; provided that not all of p, v, and u are simultaneously 0. The variable q may be independently selected from a range of between about 1 and about 12, between about 1 and about 8, or between about 1 and about 4, e.g., 1, 2, 3, or 4.

In various embodiments, the phospholipid-polymer-aromatic conjugate of Structural Formula II may be represented by:

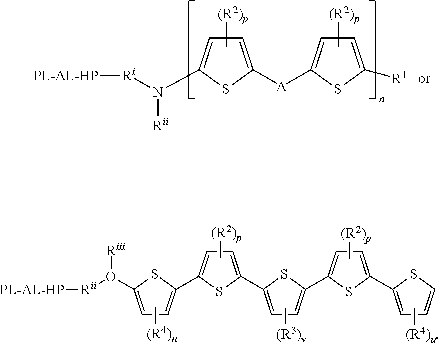

R¹ may be substituted with zero, one or more —OH. Rⁱⁱ may be C₁-C₆ alkyl substituted with zero, one or more of: —OH and alkyl optionally substituted with one or more —OH. For example, Rⁱⁱ may be C₁-C₃ alkyl or hydroxyalkyl.

Similarly, the binding ligand of Structural Formula III may be represented by:

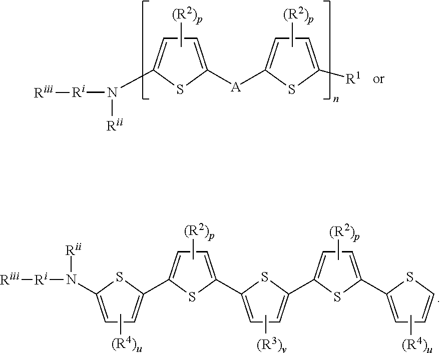

Rⁱⁱ may be hydrogen or hydroxyl. R¹ may be substituted with zero, one or more —OH. Rⁱⁱ may be C₁-C₆ alkyl substituted with zero, one or more of: —OH and alkyl optionally substituted with one or more —OH. For example, Rⁱⁱ may be C₁-C₃ alkyl or hydroxyalkyl.

### Additional Phospholipid-Polymer-Aromatic Conjugates

A variety of other binding ligands can be linked to a phospholipid-polymer to provide additional phospholipid-polymer aromatic conjugates according to Structural Formula I. In some embodiments, these phospholipid-polymer aromatic conjugates are represented by Structural Formula IV:

PL-AL-HP-X—(Ar—R¹-Het) (IV)

or a pharmaceutically acceptable salt thereof. PL may be a phospholipid. AL may be an aliphatic linkage. HP may be a hydrophilic polymer. X may be a bond, —O—, —R² O—, —R² O(C═O), R²—N(R³) O(C═O), R²—N(R³)(C═O)—, or R²—N(R³)—. R¹ may be C₂-C₆ alkyl or alkenyl. R² may be a linking group including 1 to 6 carbon atoms. R² may include one of: alkylene or alkoxyalkylene. R³ may be hydrogen, C₁-C₆ alkyl, or C₁-C₆ alkoxyalkyl. Ar may be a monocyclic or polycyclic group. Ar may include at least one aromatic or heteroaromatic ring.

Various different embodiments for the heteroaryl group (Het) are encompassed by the present invention. In some embodiments, Het may be a fused polycyclic group that contains at least one heteroaromatic ring containing at least two ring heteroatoms, wherein each ring heteroatom is nitrogen. In other embodiments, Het may be a fused polycyclic group that contains at least one heteroaromatic ring containing at least one ring heteroatom, each heteroatom being oxygen or sulfur. In some embodiments, each ring heteroatom in Het is oxygen. In yet other embodiments, Het may be a fused polycyclic group that contains at least one heteroaromatic ring containing at least two ring heteroatoms, at least one ring heteroatom being nitrogen and at least one ring heteroatom being oxygen. In further embodiments, Het may be a fused polycyclic group that contains at least one heteroaromatic ring containing at least one ring heteroatom, the at least one ring heteroatom being sulfur. In yet further embodiments, Het may be a fused polycyclic group that contains at least one heteroaromatic ring containing at least two ring heteroatoms, at least one ring heteroatom being nitrogen and at least one ring heteroatom being sulfur.

Further with regard to Structural Formula IV, X may be bonded to one of Ar or Het. The X, Ar, R¹, Het, and variables therein such as R² and R³ may further be substituted. For example, R² may be substituted with zero, one or more of: hydroxyl, C₁-C₆ alkyl, and C₁-C₆ hydroxyalkyl. Ar, Het, R¹, and R³ other than hydrogen may be independently substituted with 1, 2, or 3 of R⁶. Each R⁶ may be independently selected from —H; halogen; optionally alkylated methylenemalononitrile; —OH; —SH; alkyl; —O-alkyl; —S-alkyl; aryl; —O-aryl or —(O-alkylene)₁₋₆ optionally substituted with —OH or halogen; —NH₂; —NH-alkyl; —N-dialkyl; carboxyl; sulfonyl; carbamoyl; and glycosyl. In some embodiments, R⁶ may be —H, —OH, —SMe, or —I. The variables, e.g., X, Ar, R¹, Het, and the like may represent the same moieties in Structural Formula IV as described for Structural Formula II herein.

In some embodiments, a binding ligand represented by Structural Formula V is provided:

R⁵—(Ar—R¹-Het) (V)

or a pharmaceutically acceptable salt thereof, wherein the variables, e.g., Ar, R¹, Het, R⁵ and the like may represent the same moieties as in Structural Formula IV of the phospholipid-polymer-aromatic conjugate as described herein.

In Structural Formula V, R⁵ may be hydrogen, hydroxyl, H—R²—, HO—R²—, H—R²—N(R³)—, or HO—R²—N(R³)—. In some embodiments, R⁵ may be hydroxyl, H—R²—, HO—R²—, H—R²—N(R³)—, or HO—R²—N(R³)—. R⁵ may be H—R²—, HO—R²—, H—R²—N(R³)—, or HO—R²—N(R³)—. R⁵ may be H—R²— or H—R²—N(R³)—. R⁵ may be HO—R²— or HO—R²—N(R³)—. R⁵ may be H—R²— or HO—R²—.

In various embodiments of Structural Formulas IV and V, R¹ may be C₂ alkyl or alkenyl. For example, R¹ may be C₂-C₆ alkenyl. R¹ may be C₂-C₆ alkenyl in a trans or cis configuration, for example, trans. R¹ may be trans 1,2-ethenyl.

In some embodiments of Structural Formulas IV and V, one, two, three, or four ring atoms of the heteroaromatic rings included by Ar each independently may be one of: N, O, or S. Ar may include at least one heteroaromatic ring selected from the group consisting of: pyridine, pyrimidine, pyrazine, pyridazine, thiophene, furan, pyrrole, thiazole, oxazole, diazole, thiadiazole, oxadiazole, and triazole. Ar may include, for example, one of: phenyl, pyridine, pyrimidine, pyrazine, pyridazine, thiophene, furan, pyrrole, thiazole, oxazole, diazole, thiadiazole, oxadiazole, triazole, benzofuran, indole, benzothiophene, thienopyrimidine, benzooxazole, benzothiazole, benzooxadiazole, or benzothiadiazole. Ar may include one of phenyl or indole. Het may be one of imidazo[1,2-a]pyridine, imidazo[1,5-a]pyridine, pyrazolo[1,5-a]pyridine, pyrrolo[1,2-a]pyrimidine, pyrrolo[1,2-a]pyrazine, pyrrolo[1,2-c]pyrimidine, pyrrolo[1,2-b]pyridazine, quinazoline, quinoxaline, 1,5-naphthyridine, 1,6-naphthyridine, 1,7-naphthyridine, or 1,8-naphthyridine. Het may be one of quinazoline, quinoxaline, 1,5-naphthyridine, 1,6-naphthyridine, 1,7-naphthyridine, or 1,8-naphthyridine. Het may be one of imidazo[1,2-a]pyridine, imidazo[1,5-a]pyridine, or pyrazolo[1,5-a]pyridine. Het may be one of pyrrolo[1,2-a]pyrimidine, pyrrolo[1,2-a]pyrazine, pyrrolo[1,2-c]pyrimidine, or pyrrolo[1,2-b]pyridazine, Het may be imidazo[1,2-a]pyridine.

In various embodiments of Structural Formulas IV and V, each amine and heteroaromatic ring nitrogen may independently and optionally be alkylated to form a quaternary ammonium accompanied by a pharmaceutically acceptable anion, e.g., a halide ion, an acetate ion, and the like. For example, each amine, thiazole, and benzothiazole nitrogen may independently and optionally be alkylated to form a quaternary ammonium accompanied by a pharmaceutically acceptable anion, e.g., a halide ion, an acetate ion, and the like.

In several embodiments of Structural Formulas IV and V, Ar and Het may be independently substituted with zero, one or more of: F, Cl, Br, I, alkyl, aryl, —OH, —O-alkyl, —O-aryl, —NH₂, —NH-alkyl, —N-dialkyl, carboxyl, sulfonyl, carbamoyl, and glycosyl.

In various embodiments of Structural Formulas IV and V, the phospholipid-polymer-aromatic conjugate may be represented by PL-AL-HP—O—(Ar—R¹-Het). The compound may be represented by H—O—(Ar—R¹-Het). Het and/or Ar may be substituted by —O-alkyl. Het and/or Ar may be substituted by methoxy.

Examples of compounds in which Het is a fused polycyclic group that contains at least one heteroaromatic ring containing at least two ring heteroatoms, wherein each ring heteroatom is nitrogen, are shown below. In some embodiments of Structural Formula IV, the phospholipid-polymer-aromatic conjugate may be represented by:

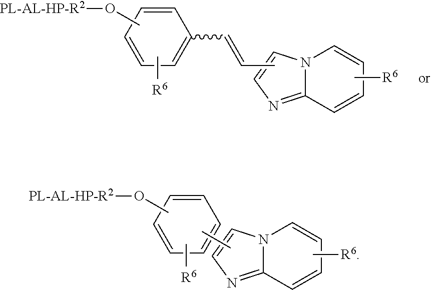

Similarly, the compound of Structural Formula V may be represented by:

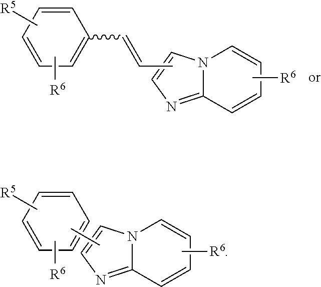

R⁵ may be H, hydroxyl, H—R²—, HO—R²—, H—R²—N(R³)—, or HO—R²—N(R³)—. R⁶ may independently be —H, —OH, —O-alkyl, —S-alkyl, —NH₂, or —I.

In some embodiments, the phospholipid-polymer-aromatic conjugate may be represented by:

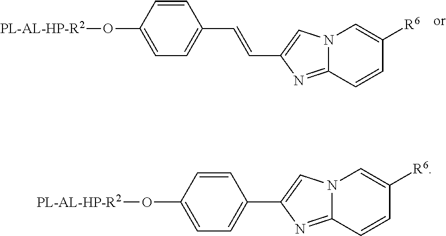

Similarly, the compound of Structural Formula V may be represented by:

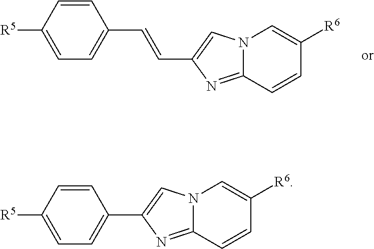

R⁵ may be H, hydroxyl, H—R²—, HO—R²—, H—R²—N(R³)—, or HO—R²—N(R³)—. In some embodiments, R⁵ may be hydroxyl. Each R⁶ may independently be —H, —OH, —O-alkyl, —S-alkyl, —NH₂, or —I. In several embodiments, the phospholipid-polymer-aromatic conjugate may be represented by:

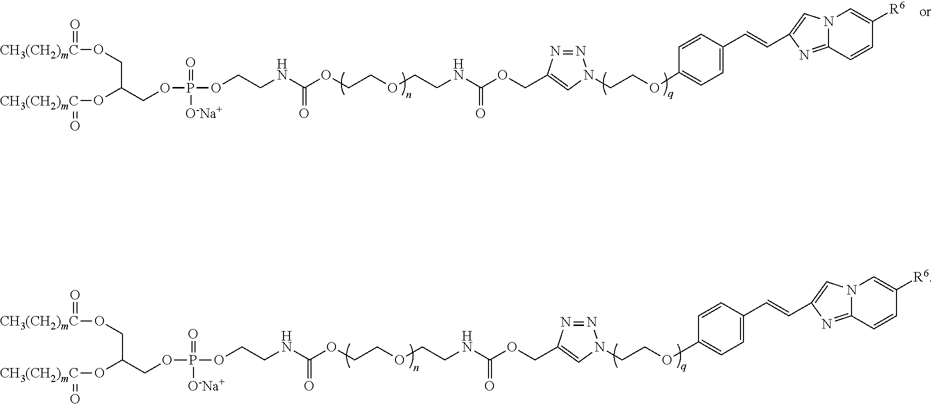

The variable n may be any integer from about 10 to about 100, for example, about 60 to about 100, about 70 to about 90, about 75 to about 85, about 77, and the like. The variable m may be one of: 12, 13, 14, 15, 16, 17, or 18. The variable q may be independently selected from a range of between about 1 and about 12, between about 1 and about 8, or between about 1 and about 4, e.g., 1, 2, 3, or 4. For example, n may be 77, q may be 4, and m may be 14. In another example, n may be 77, q may be 1, and m may be 16. Each R⁶ may independently be —H, —OH, —O-alkyl, —S-alkyl, —NH₂, or —I.

In various embodiments, the phospholipid-polymer-aromatic conjugate may be represented by PL-AL-HP—R²—N(R³)—(Ar—R¹-Het). Ar may be unsubstituted. Ar may be monocyclic. Ar may include a carbocyclic aromatic ring, for example, Ar may be a phenyl ring. Ar may be indole. For example, Ar may be unsubstituted 1,4-phenylene or unsubstituted 1,5-indolyl. R² may be substituted with zero, one or more —OH. R³ may be C₁-C₆ alkyl substituted with zero, one or more of: —OH and alkyl optionally substituted with one or more —OH. For example, R³ may be C₁-C₃ alkyl or hydroxyalkyl.

In some embodiments, the phospholipid-polymer-aromatic conjugate may be represented by:

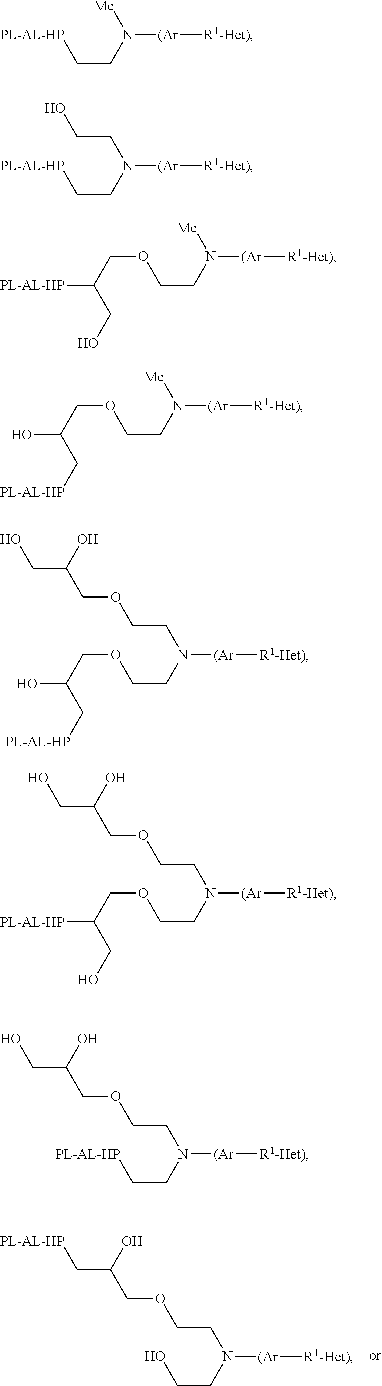

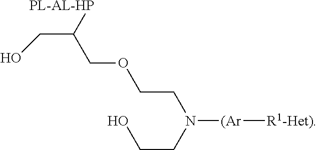

The binding ligand (Ar—R¹-Het) may be bonded to the rest of the phospholipid-polymer-aromatic conjugate by Ar or Het, e.g., by Ar.

In some embodiments of Structural Formula IV, the phospholipid-polymer-aromatic conjugate may be represented by:

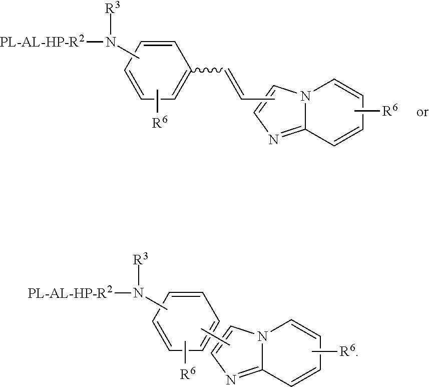

Similarly, the compound of Structural Formula V may be represented by:

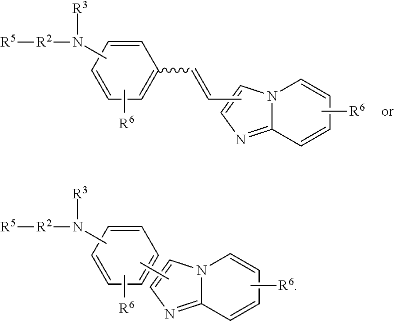

R⁵ may be H, hydroxyl, H—R²—, HO—R²—, H—R²—N(R³)—, or HO—R²—N(R³)—. Each R⁶ may independently be —H, —OH, —O-alkyl, —S-alkyl, —NH₂, or —I.

In some embodiments of Structural Formula IV, the phospholipid-polymer-aromatic conjugate may be represented by:

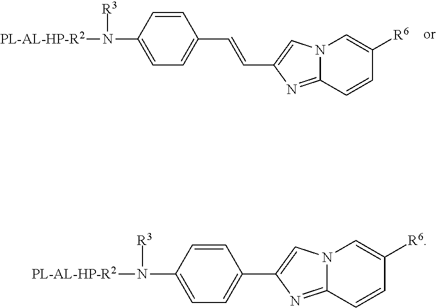

Similarly, the compound of Structural Formula V may be represented by:

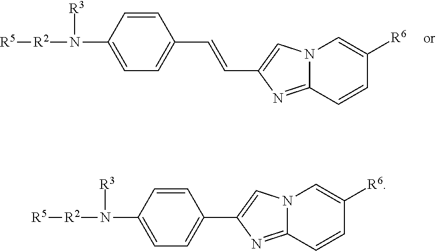

R⁵ may be H, hydroxyl, H—R²—, HO—R²—, H—R²—N(R³)—, or HO—R²—N(R³)—. Each R⁶ may independently be —H, —OH, —O-alkyl, —S-alkyl, —NH₂, or —I.

In several embodiments, the phospholipid-polymer-aromatic conjugate may be represented by one of:

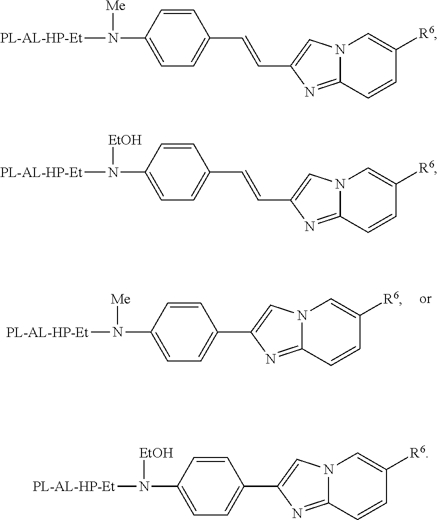

Each R⁶ may independently be —H, —OH, —O-alkyl, —S-alkyl, —NH₂, or —I.

In various embodiments, the phospholipid-polymer-aromatic conjugate may be represented by one of:

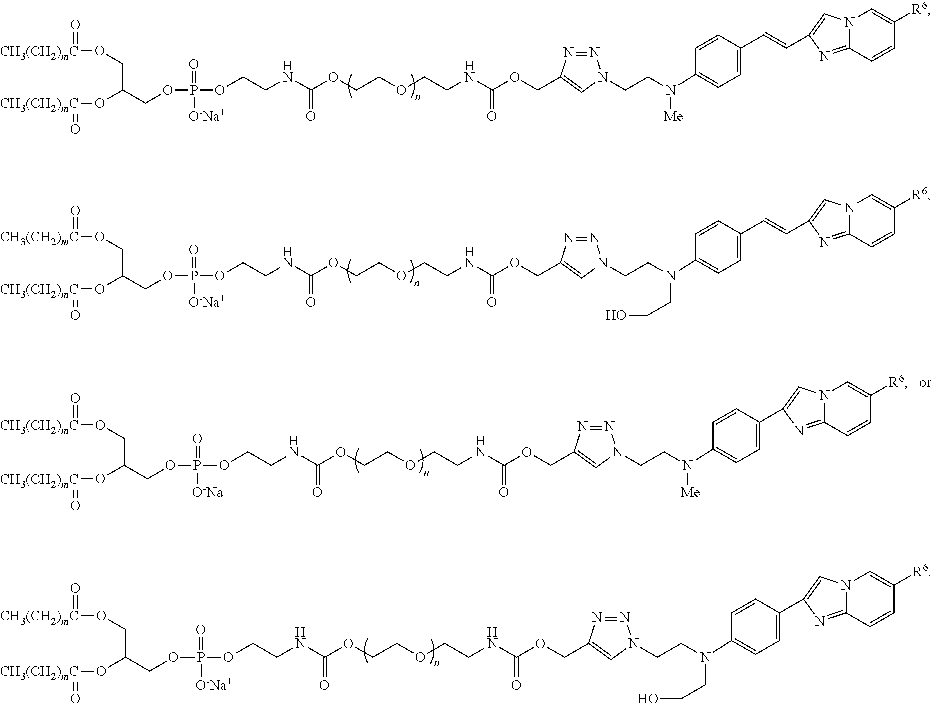

The variable n may be any integer from about 10 to about 100, for example, about 60 to about 100, about 70 to about 90, about 75 to about 85, or about 77. The variable m may be one of: 12, 13, 14, 15, 16, 17, or 18. For example, n may be 77 and m may be 14. In another example, n may be 77 and m may be 16. Each R⁶ may independently be —H, —OH, —O-alkyl, —S-alkyl, —NH₂, or —I.

In various embodiments, the phospholipid-polymer-aromatic conjugate may be represented by one of:

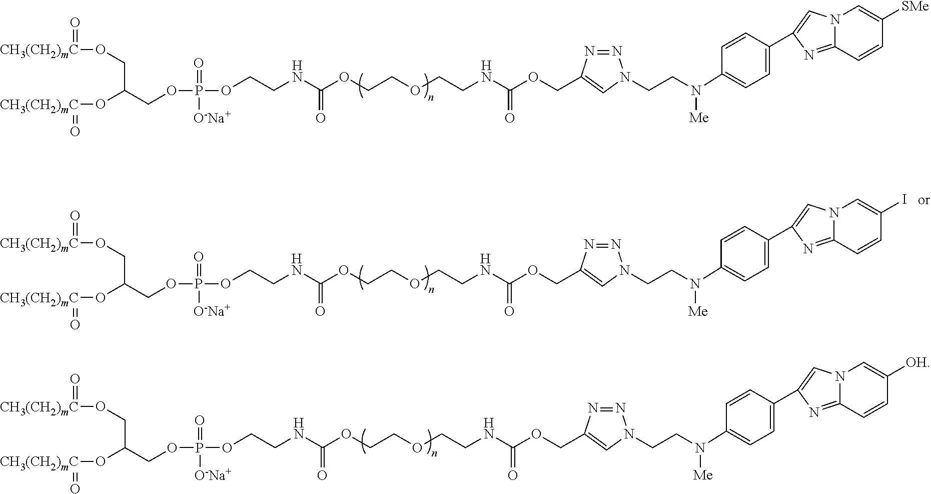

The variable n may be any integer from about 10 to about 100, for example, about 60 to about 100, about 70 to about 90, about 75 to about 85, or about 77. The variable m may be one of: 12, 13, 14, 15, 16, 17, or 18. For example, n may be 77 and m may be 14. In another example, n may be 77 and m may be 16.

Examples of conjugates and binding ligands in which Het is a fused polycyclic group that contains at least one heteroaromatic ring containing at least one ring heteroatom, each heteroatom being oxygen or sulfur are provided below. In some embodiments, each ring heteroatom in Het is oxygen. In some embodiments of Structural Formula IV, the phospholipid-polymer-aromatic conjugate may be represented by:

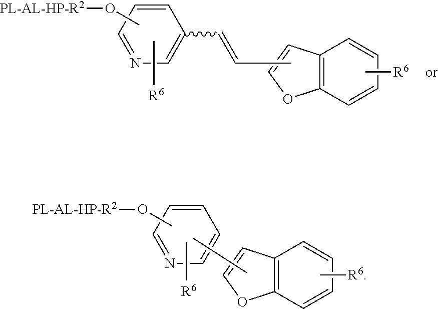

Similarly, the binding ligands of Structural Formula V may be represented by:

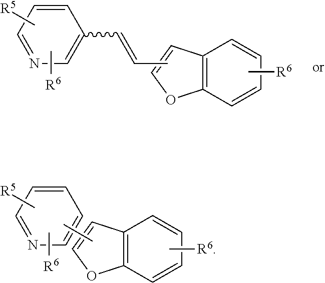

R⁵ may be H, hydroxyl, H—R²—, HO—R²—, H—R²—N(R³)—, or HO—R²—N(R³)—. R⁶ may independently be —H, —OH, —O-alkyl, —S-alkyl, —NH₂, or —F.

In some embodiments, the phospholipid-polymer-aromatic conjugate may be represented by:

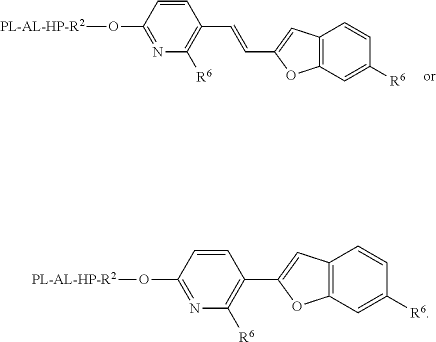

Similarly, the compound of Structural Formula V may be represented by:

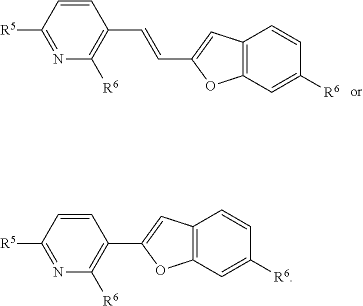

R⁵ may be H, hydroxyl, H—R²—, HO—R²—, H—R²—N(R³)—, or HO—R²—N(R³)—. In some embodiments, R⁵ may be hydroxyl. Each R⁶ may independently be —H, —OH, —O-alkyl, —S-alkyl, —NH₂, or —F. In several embodiments, the phospholipid-polymer-aromatic conjugate may be represented by:

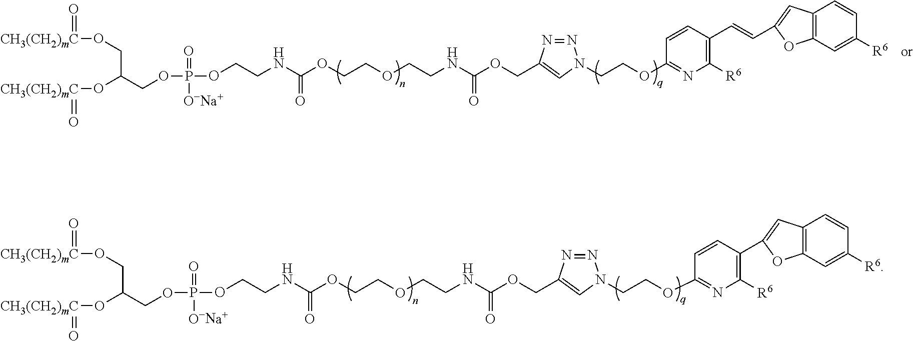

The variable n may be any integer from about 10 to about 100, for example, about 60 to about 100, about 70 to about 90, about 75 to about 85, about 77, and the like. The variable m may be one of: 12, 13, 14, 15, 16, 17, or 18. The variable q may be independently selected from a range of between about 1 and about 12, between about 1 and about 8, or between about 1 and about 4, e.g., 1, 2, 3, or 4. For example, n may be 77, q may be 4, and m may be 14. In another example, n may be 77, q may be 1, and m may be 16. Each R⁶ may independently be —H, —OH, —O-alkyl, —S-alkyl, —NH₂, or —F.

In some embodiments, the phospholipid-polymer-aromatic conjugate may be represented by:

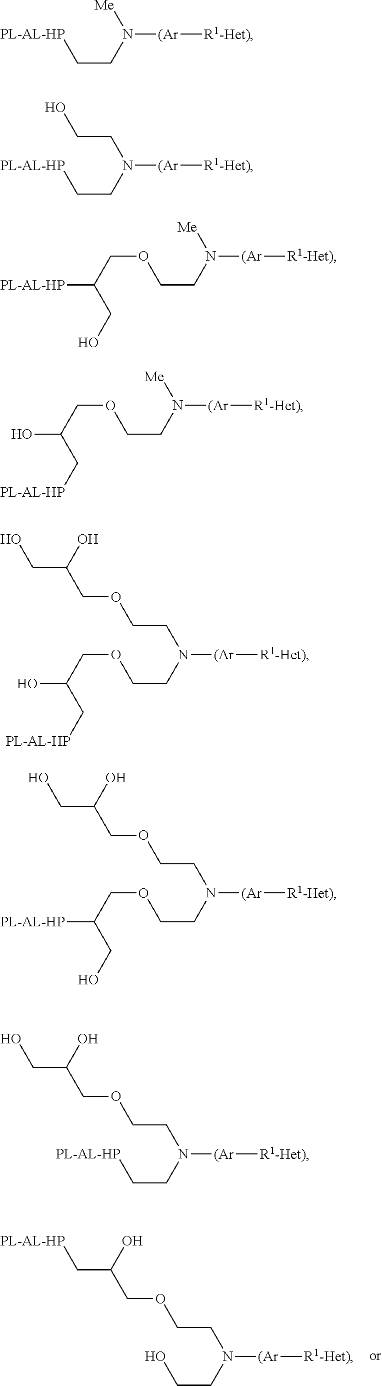

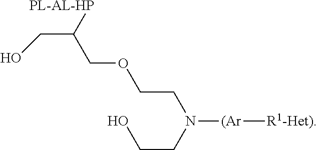

The group (Ar—R¹-Het) may be bonded to the rest of the phospholipid-polymer-aromatic conjugate by Ar or Het, e.g., by Ar.

In some embodiments of Structural Formula IV, the phospholipid-polymer-aromatic conjugate may be represented by:

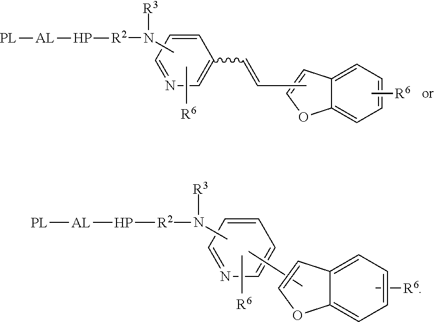

Similarly, the compound of Structural Formula V may be represented by:

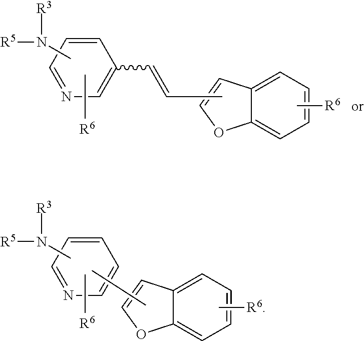

R⁵ may be H, hydroxyl, H—R²—, HO—R²—, H—R²—N(R³)—, or HO—R²—N(R³)—. Each R⁶ may independently be —H, —OH, —O-alkyl, —S-alkyl, —NH₂, or —F.

In some embodiments of Structural Formula IV, the phospholipid-polymer-aromatic conjugate may be represented by:

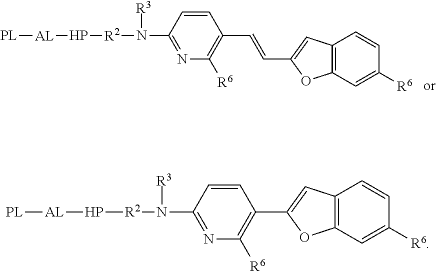

Similarly, binding ligands of Structural Formula V may be represented by:

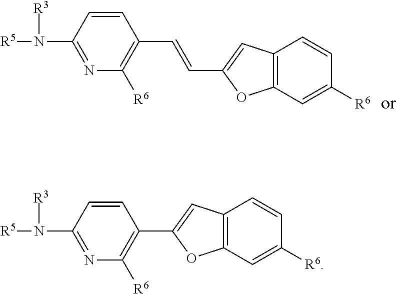

R⁵ may be H, hydroxyl, H—R²—, HO—R²—, H—R²—N(R³)—, or HO—R²—N(R³)—. Each R⁶ may independently be —H, —OH, —O-alkyl, —S-alkyl, —NH₂, or —F.

In several embodiments, the phospholipid-polymer-aromatic conjugate may be represented by one of:

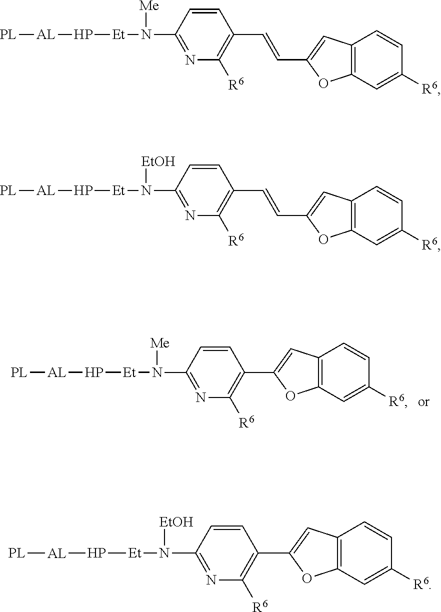

Each R⁶ may independently be —H, —OH, —O-alkyl, —S-alkyl, —NH₂, or —F.

In various embodiments, the phospholipid-polymer-aromatic conjugate may be represented by one of:

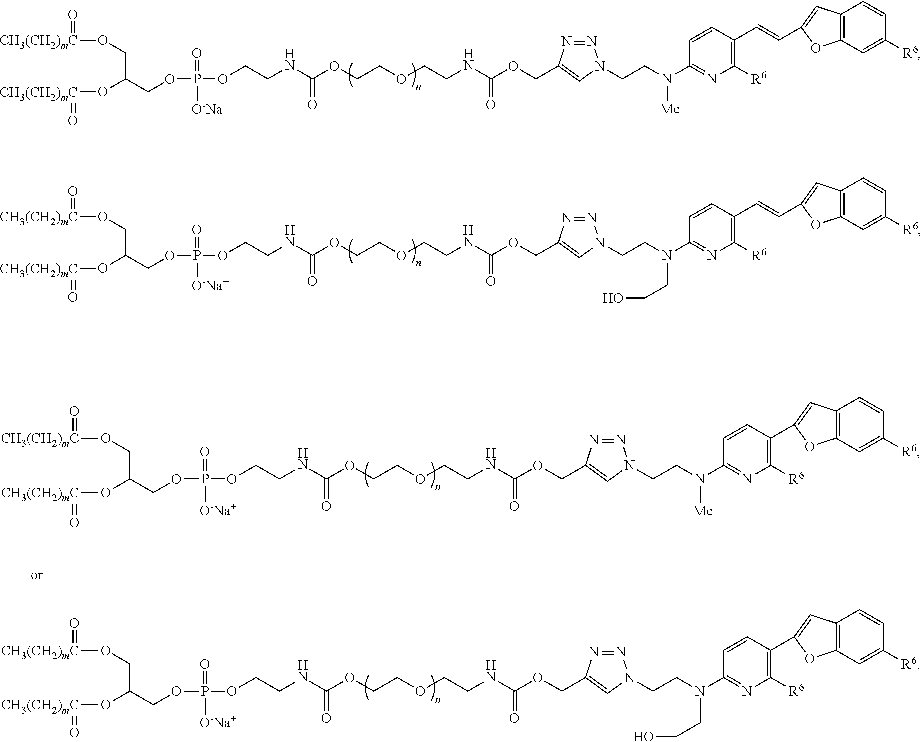

The variable n may be any integer from about 10 to about 100, for example, about 60 to about 100, about 70 to about 90, about 75 to about 85, or about 77. The variable m may be one of: 12, 13, 14, 15, 16, 17, or 18. For example, n may be 77 and m may be 14. In another example, n may be 77 and m may be 16. Each R⁶ may independently be —H, —OH, —O-alkyl, —S-alkyl, —NH₂, or —F, for example, the conjugate may be one of:

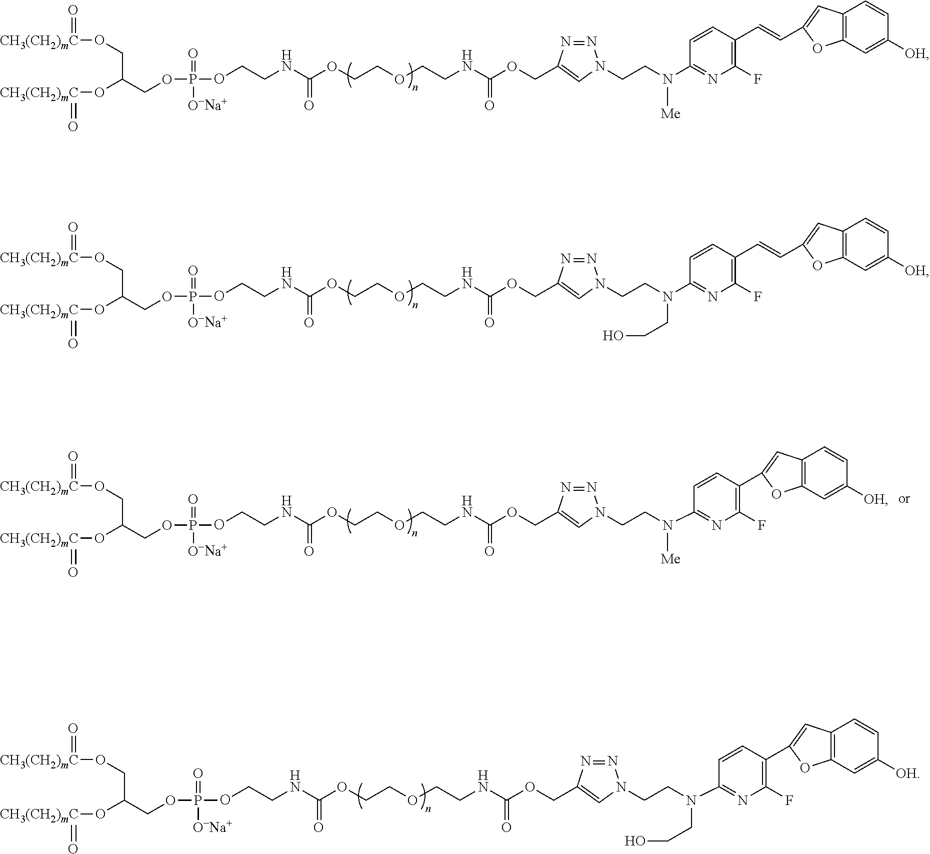

Examples of conjugates and binding ligands in which Het is a fused polycyclic group that contains at least one heteroaromatic ring containing at least two ring heteroatoms, at least one ring heteroatom being nitrogen and at least one ring heteroatom being oxygen are provided below. In some embodiments of Structural Formula IV, the phospholipid-polymer-aromatic conjugate may be represented by:

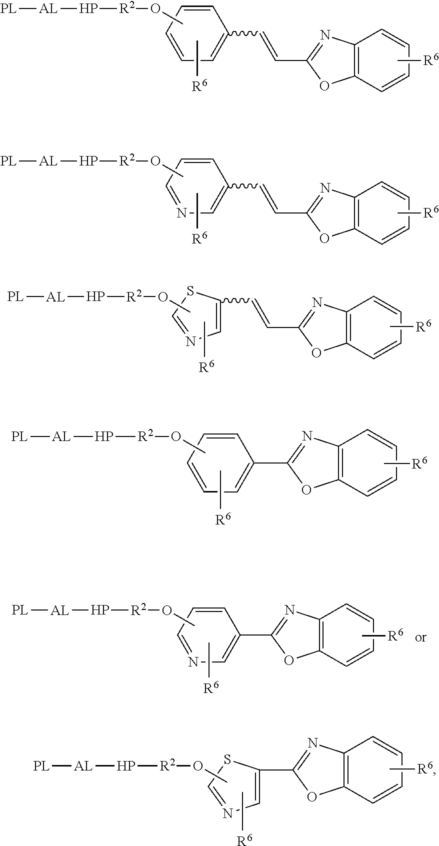

Similarly, the compound of Structural Formula V may be represented by:

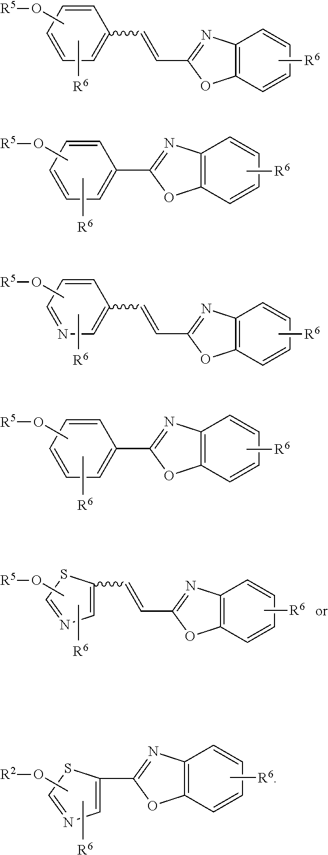

R⁵ may be H, hydroxyl, H—R²—, HO—R²—, H—R²—N(R³)—, or HO—R²—N(R³)—. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments, the phospholipid-polymer-aromatic conjugate may be represented by:

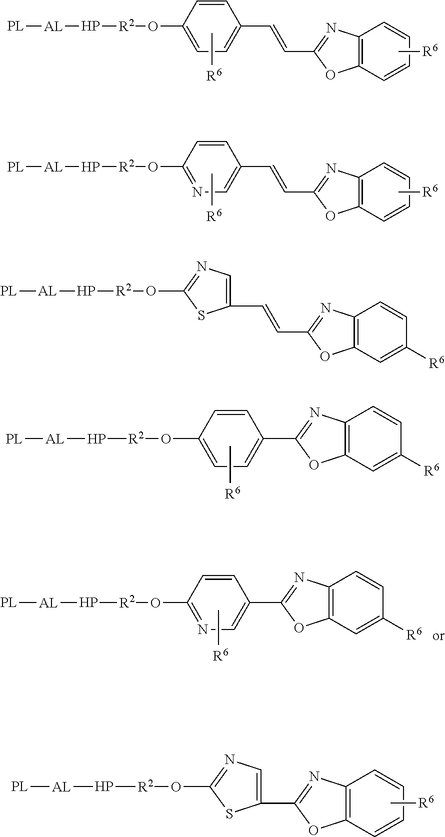

Similarly, binding ligands of Structural Formula V may be represented by:

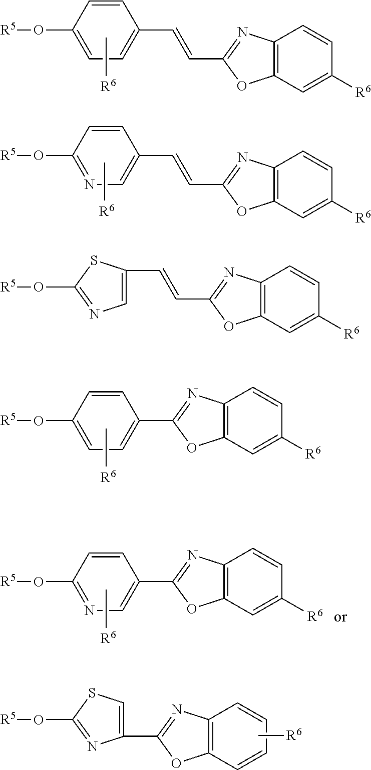

R⁵ may be H, hydroxyl, H—R²—, HO—R²—, H—R²—N(R³)—, or HO—R²—N(R³)—. In some embodiments, R⁵ may be hydroxyl. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like. In several embodiments, the phospholipid-polymer-aromatic conjugate may be represented by:

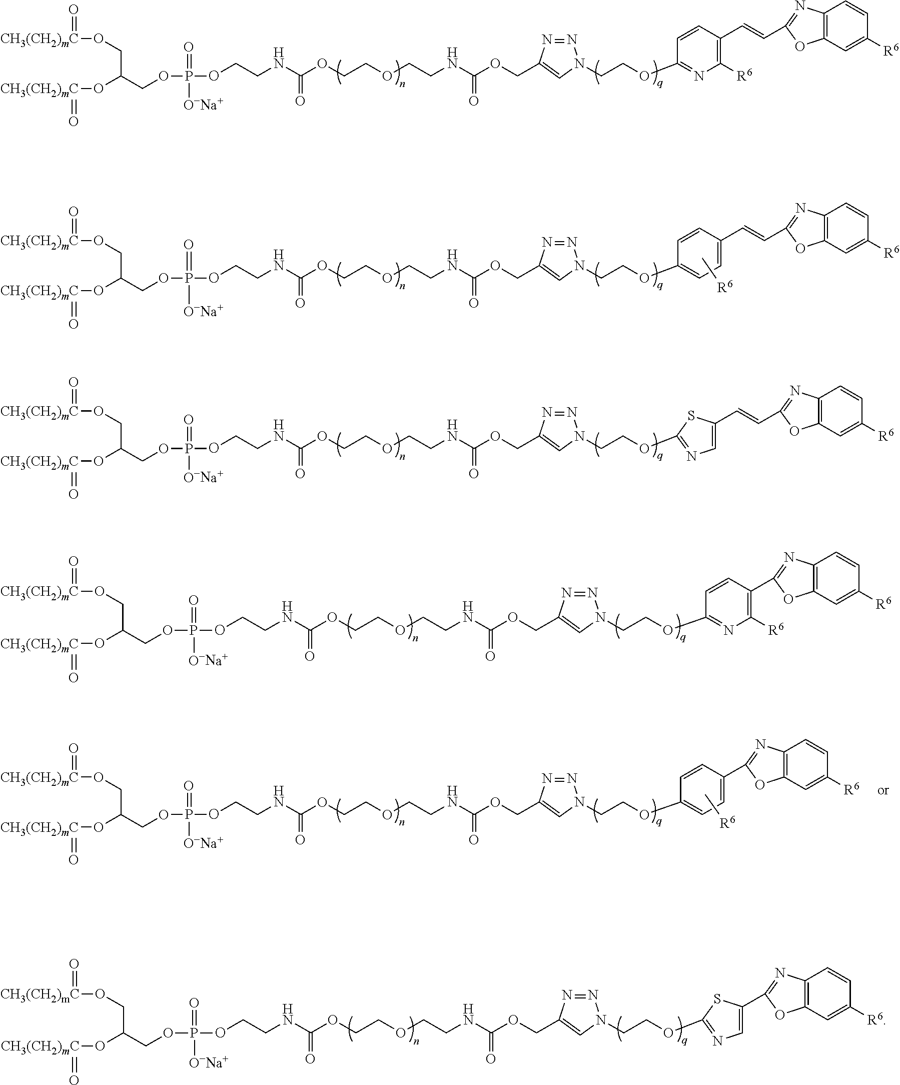

The variable n may be any integer from about 10 to about 100, for example, about 60 to about 100, about 70 to about 90, about 75 to about 85, about 77, and the like. The variable m may be one of: 12, 13, 14, 15, 16, 17, or 18. The variable q may be independently selected from a range of between about 1 and about 12, between about 1 and about 8, or between about 1 and about 4, e.g., 1, 2, 3, or 4. For example, n may be 77, q may be 4, and m may be 14. In another example, n may be 77, q may be 1, and m may be 16. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments, the phospholipid-polymer-aromatic conjugate may be represented by:

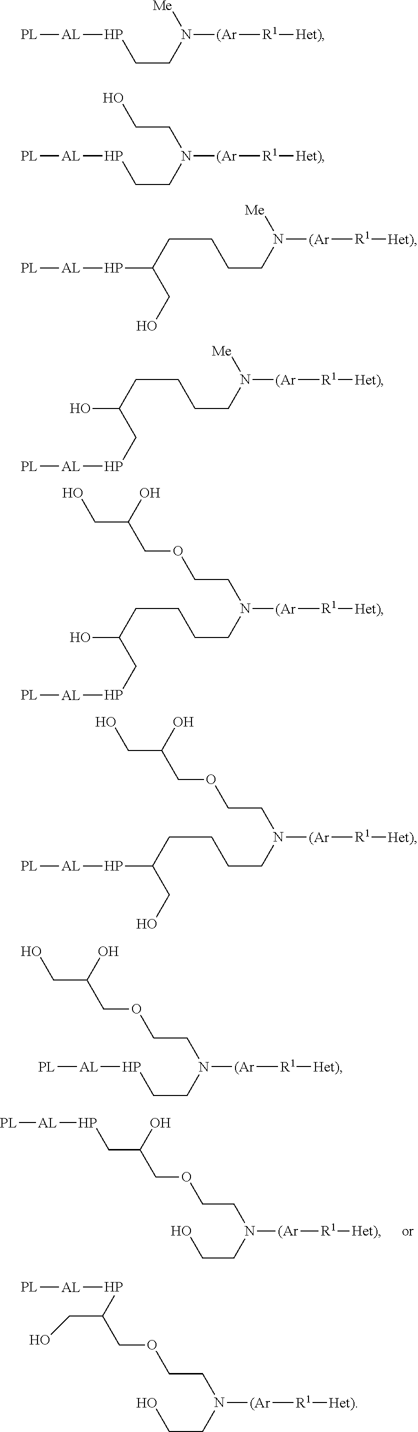

The group (Ar—R¹-Het) may be bonded to the rest of the phospholipid-polymer-aromatic conjugate by Ar or Het, e.g., by Ar.

In some embodiments of Structural Formula IV, the phospholipid-polymer-aromatic conjugate may be represented by:

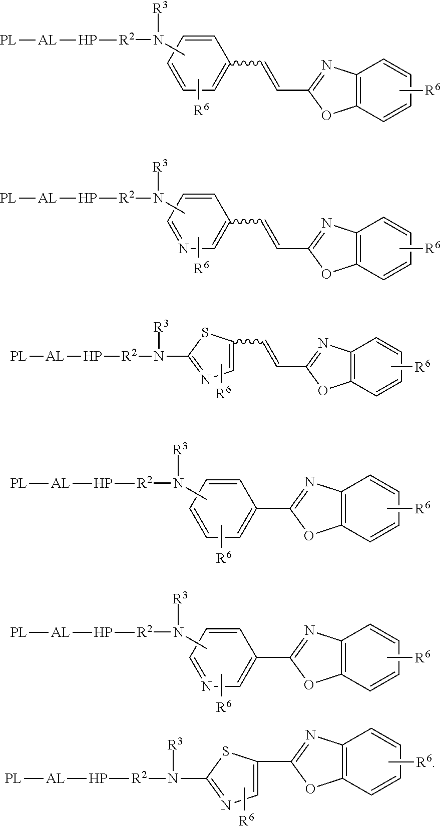

Similarly, the compound of Structural Formula V may be represented by:

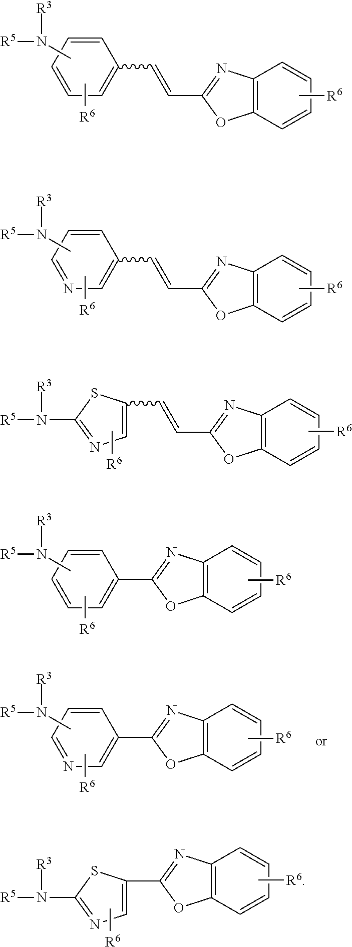

R⁵ may be H, hydroxyl, H—R²—, HO—R²—, H—R²—N(R³)—, or HO—R²—N(R³)—. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments of Structural Formula IV, the phospholipid-polymer-aromatic conjugate may be represented by:

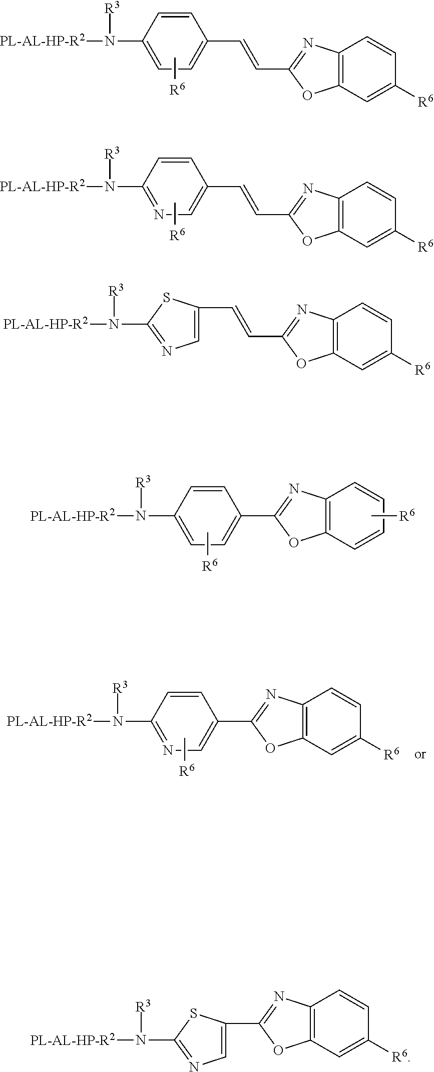

Similarly, the compound of Structural Formula V may be represented by:

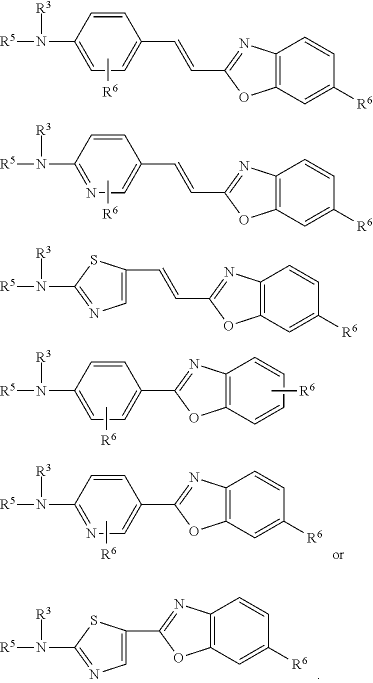

R⁵ may be H, hydroxyl, H—R²—, HO—R²—, H—R²—N(R³)—, or HO—R²—N(R³)—. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In several embodiments, the phospholipid-polymer-aromatic conjugate may be represented by one of:

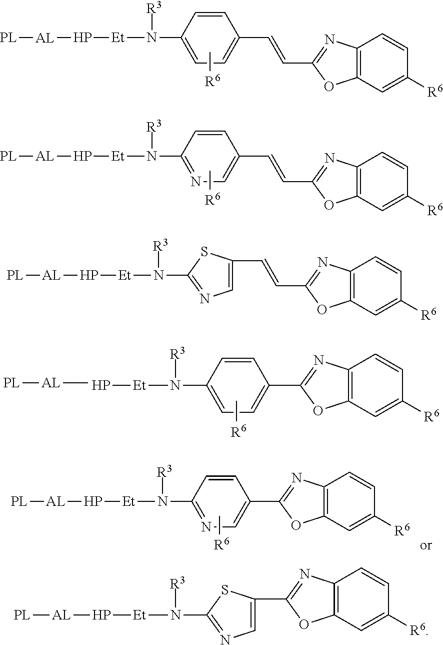

Each R⁶ may independently be —H, —OH, alkyl, —O-alkyl, —F, or —O-fluoroalkyl. Each R³ may independently be H, Me, or EtOH. Each amine, thiazole, and benzothiazole nitrogen may independently and optionally be alkylated to form a quaternary ammonium accompanied by a pharmaceutically acceptable anion, e.g., a halide ion, an acetate ion, and the like.

In various embodiments, the phospholipid-polymer-aromatic conjugate may be represented by one of:

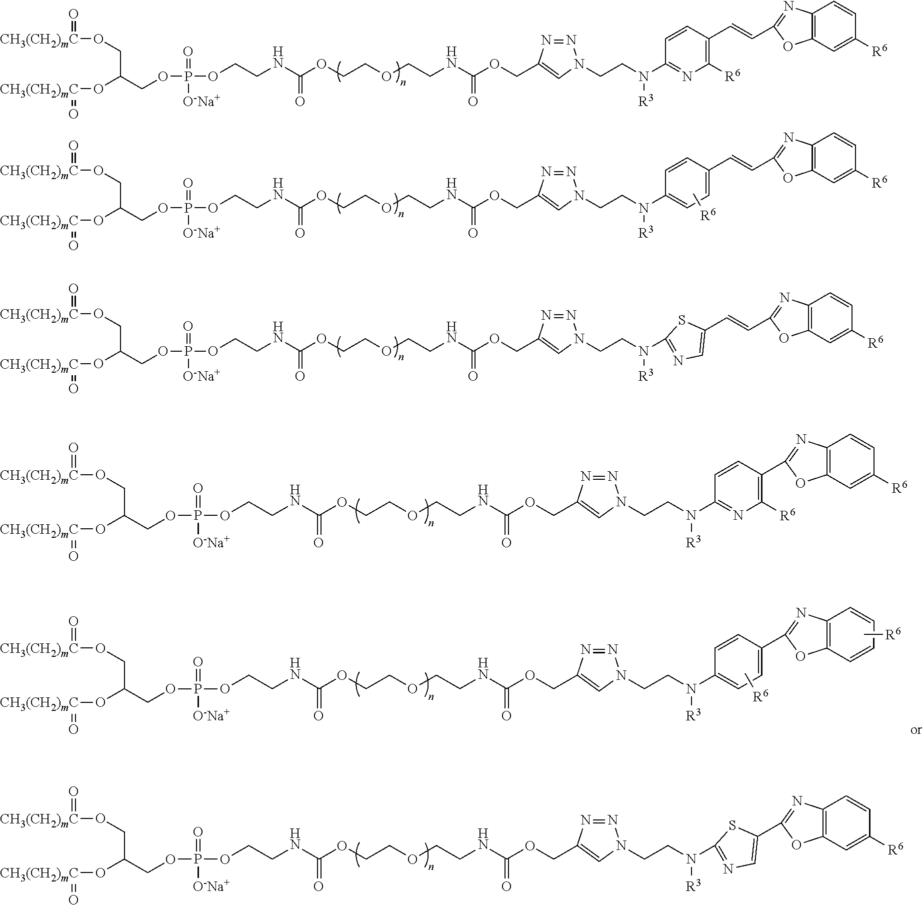

The variable n may be any integer from about 10 to about 100, for example, about 60 to about 100, about 70 to about 90, about 75 to about 85, or about 77. The variable m may be one of: 12, 13, 14, 15, 16, 17, or 18. For example, n may be 77 and m may be 14. In another example, n may be 77 and m may be 16. Each R⁶ may be independently be —H, —OH, alkyl, —O-alkyl, —F or —O-fluoroalkyl. Each R³ may independently be H, Me, or EtOH. Each amine, thiazole, and benzothiazole nitrogen may independently and optionally be alkylated to form a quaternary ammonium accompanied by a pharmaceutically acceptable anion, e.g., a halide ion, an acetate ion, and the like. For example, the conjugate may be one of:

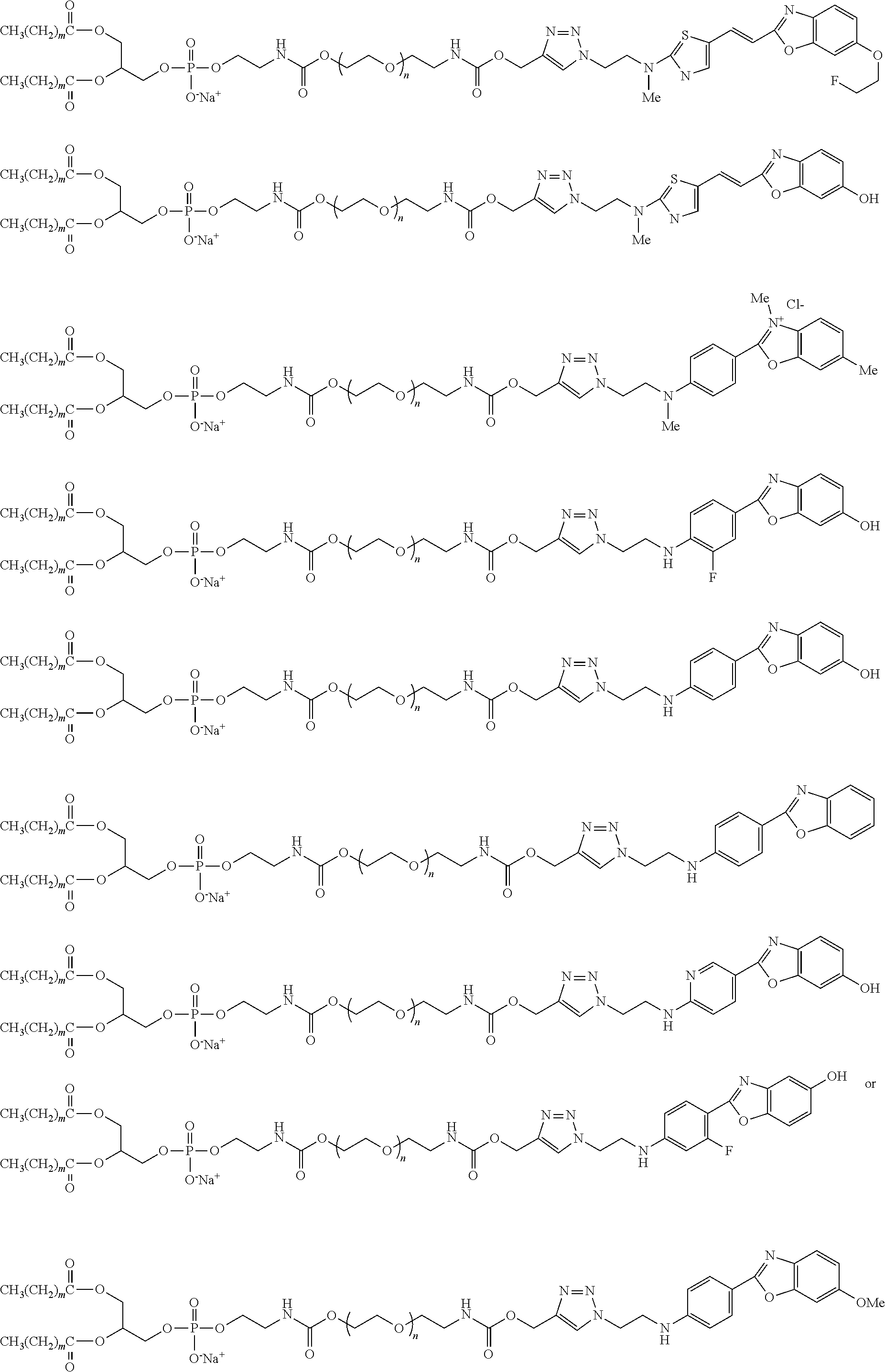

Examples of conjugates and binding ligands in which Het is a fused polycyclic group that contains at least one heteroaromatic ring containing at least one ring heteroatom, the at least one ring heteroatom being sulfur are provided below. In some embodiments of Structural Formula IV, the phospholipid-polymer-aromatic conjugate may be represented by:

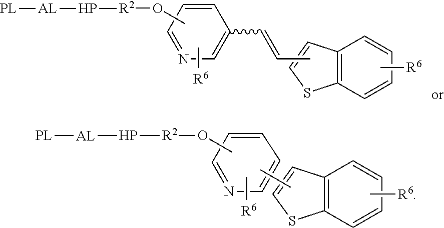

Similarly, the compound of Structural Formula V may be represented by:

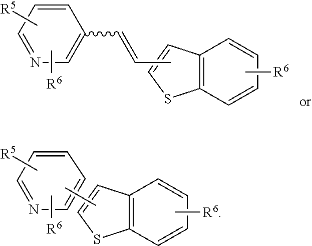

R⁵ may be H, hydroxyl, H—R²— HO—Rⁱ—, H—R²—N(R³)—, or HO—R²—N(R³)—. R⁶ may independently be —H, —OH, —O-alkyl, —S-alkyl, —NH₂, or —F.

In some embodiments, the phospholipid-polymer-aromatic conjugate may be represented by:

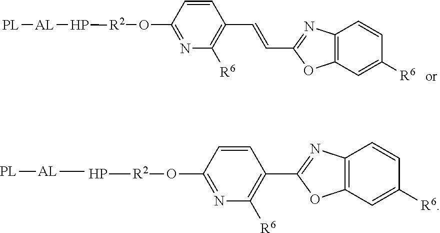

Similarly, the compound of Structural Formula V may be represented by:

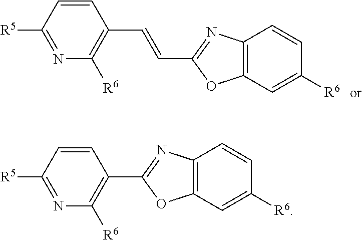

R⁵ may be H, hydroxyl, H—R²—, HO—R²—, H—R²—N(R³)—, or HO—R²—N(R³)—. In some embodiments, R⁵ may be hydroxyl. Each R⁶ may independently be —H, —OH, —O-alkyl, —S-alkyl, —NH₂, or —F. In several embodiments, the phospholipid-polymer-aromatic conjugate may be represented by:

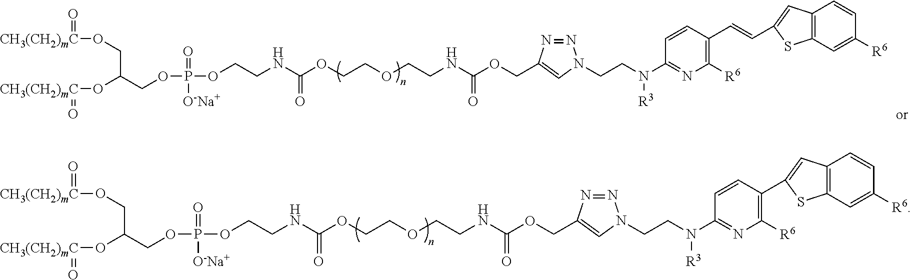

The variable n may be any integer from about 10 to about 100, for example, about 60 to about 100, about 70 to about 90, about 75 to about 85, about 77, and the like. The variable m may be one of: 12, 13, 14, 15, 16, 17, or 18. The variable q may be independently selected from a range of between about 1 and about 12, between about 1 and about 8, or between about 1 and about 4, e.g., 1, 2, 3, or 4. For example, n may be 77, q may be 4, and m may be 14. In another example, n may be 77, q may be 1, and m may be 16. Each R⁶ may independently be —H, —OH, —O-alkyl, —S-alkyl, —NH₂, or —F.

In some embodiments, the phospholipid-polymer-aromatic conjugate may be represented by:

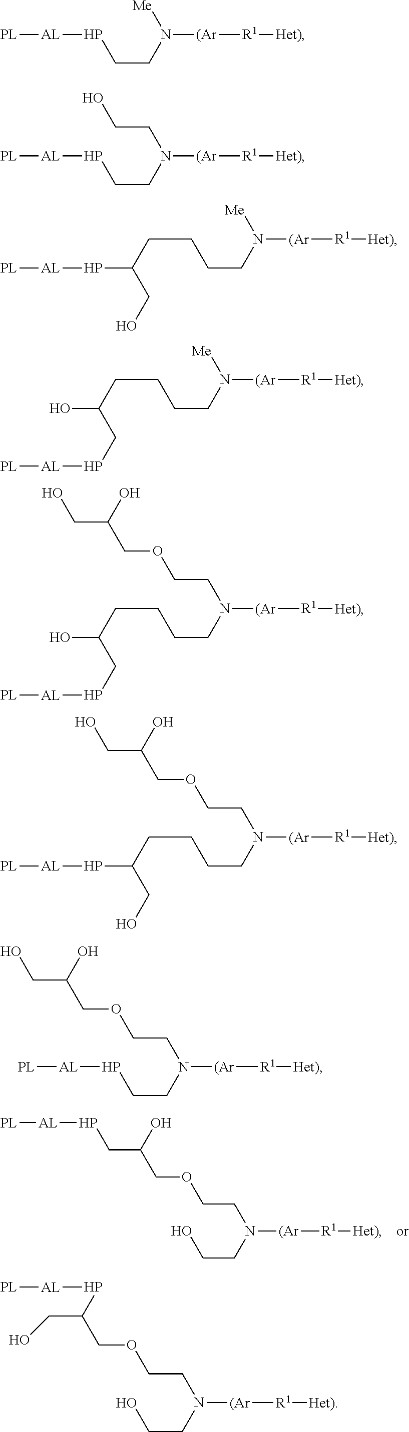

The binding ligand (Ar—R¹-Het) may be bonded to the rest of the phospholipid-polymer-aromatic conjugate by Ar or Het, e.g., by Ar.

In some embodiments of Structural Formula IV, the phospholipid-polymer-aromatic conjugate may be represented by:

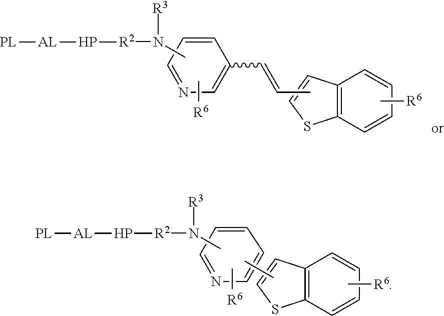

Similarly, the compound of Structural Formula V may be represented by:

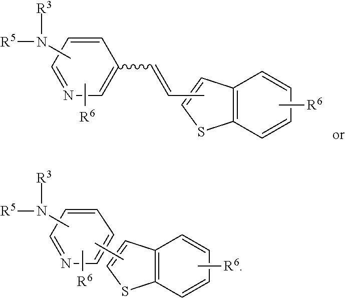

R⁵ may be H, hydroxyl, H—R²—, HO—R²—, H—R²—N(R³)—, or HO—R²—N(R³)—. Each R⁶ may independently be —H, —OH, —O-alkyl, —S-alkyl, —NH₂, or —F.

In some embodiments of Structural Formula IV, the phospholipid-polymer-aromatic conjugate may be represented by:

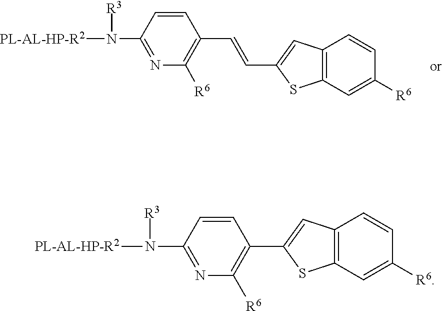

Similarly, the binding ligand of Structural Formula V may be represented by:

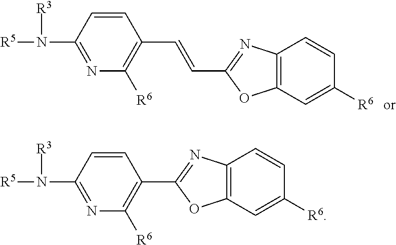

R⁵ may be H, hydroxyl, H—R²—, HO—R²—, H—R²—N(R³)—, or HO—R²—N(R³)—. Each R⁶ may independently be —H, —OH, —O-alkyl, —S-alkyl, —NH₂, or —F.

In several embodiments, the phospholipid-polymer-aromatic conjugate may be represented by one of:

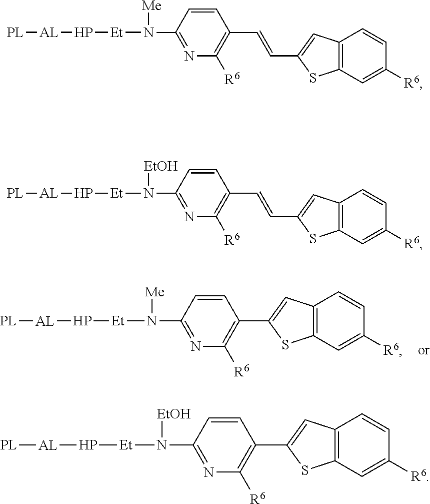

Each R⁶ may independently be —H, —OH, —O-alkyl, —S-alkyl, —NH₂, or —F.

In various embodiments, the phospholipid-polymer-aromatic conjugate may be represented by one of:

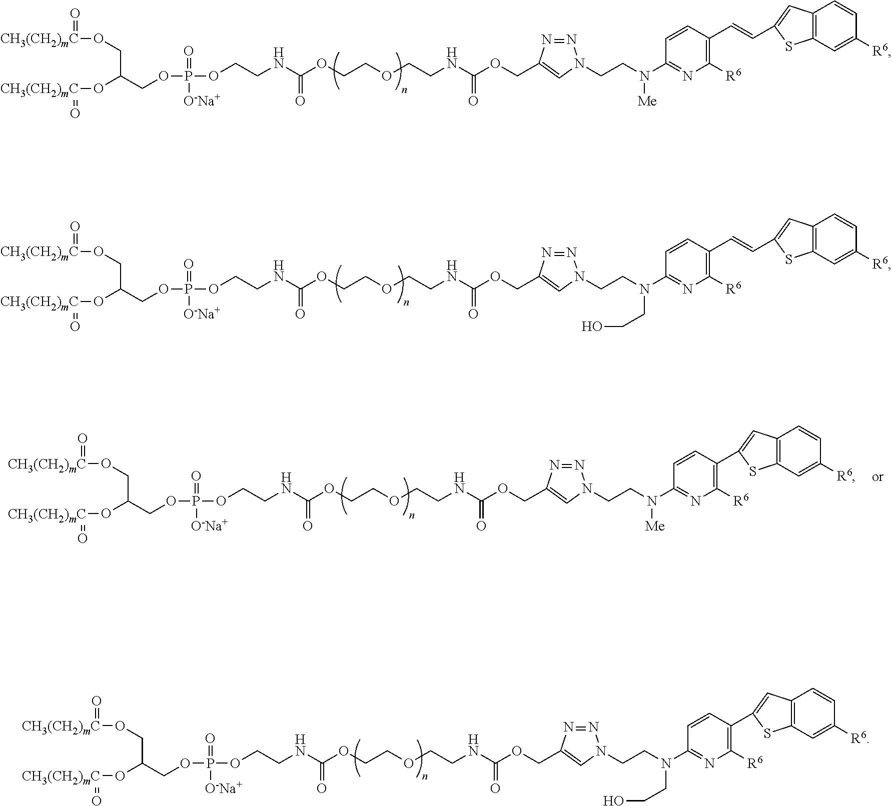

The variable n may be any integer from about 10 to about 100, for example, about 60 to about 100, about 70 to about 90, about 75 to about 85, or about 77. The variable m may be one of: 12, 13, 14, 15, 16, 17, or 18. For example, n may be 77 and m may be 14. In another example, n may be 77 and m may be 16. Each R⁶ may independently be —H, —OH, —O-alkyl, —S-alkyl, —NH₂, or —F, for example, the conjugate may be one of:

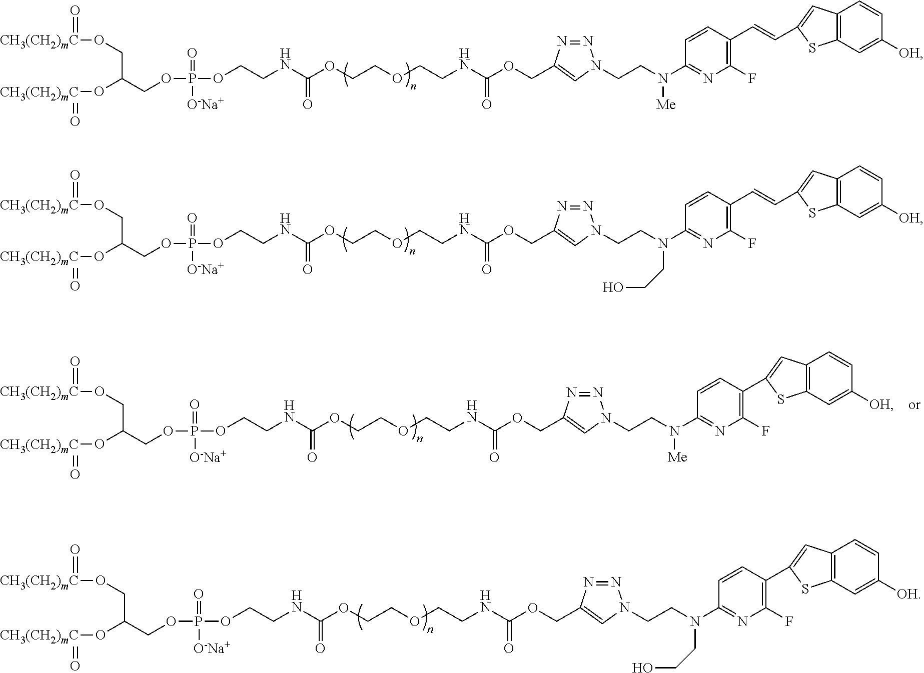

Examples of conjugates and binding ligands in which Het may be a fused polycyclic group that contains at least one heteroaromatic ring containing at least two ring heteroatoms, at least one ring heteroatom being nitrogen and at least one ring heteroatom being sulfur are provided below. In some embodiments of Structural Formula IV, the phospholipid-polymer-aromatic conjugate may be represented by:

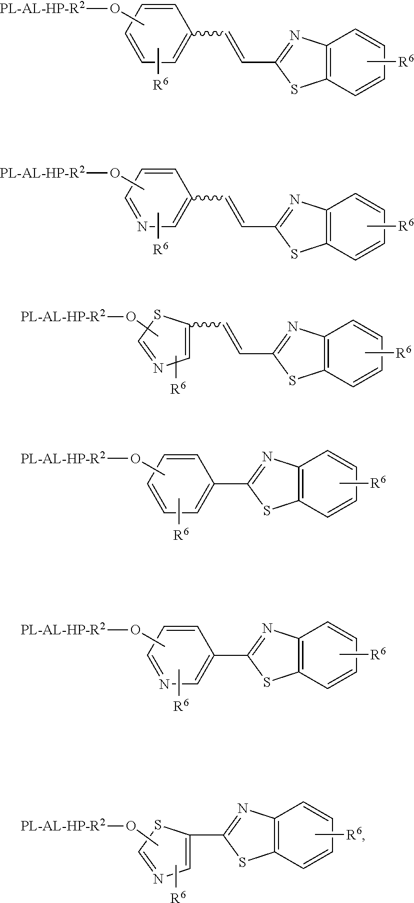

Similarly, the compound of Structural Formula V may be represented by:

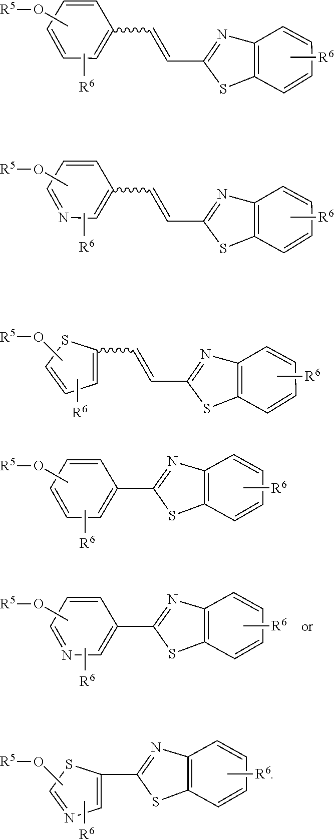

R⁵ may be H, hydroxyl, H—R²—, HO—R²—, H—R²—N(R³)—, or HO—R²—N(R³)—. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments, the phospholipid-polymer-aromatic conjugate may be represented by:

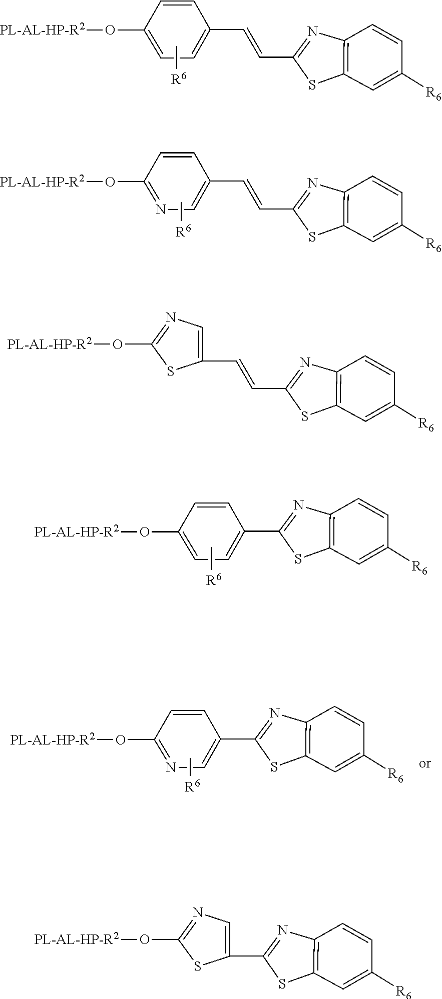

Similarly, the compound of Structural Formula V may be represented by:

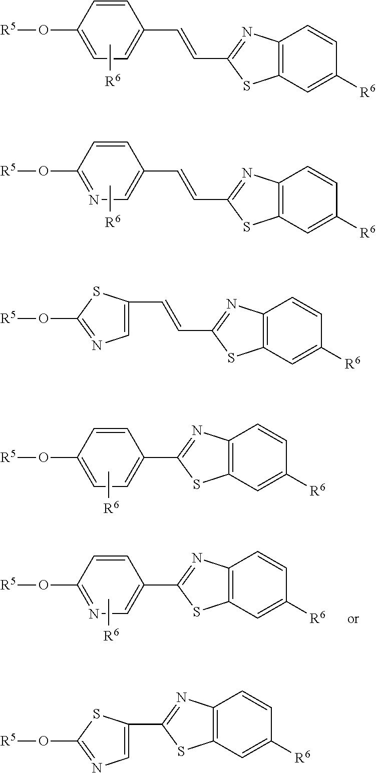

R⁵ may be H, hydroxyl, H—R²—, HO—R²—, H—R²—N(R³)—, or HO—R²—N(R³)—. In some embodiments, R⁵ may be hydroxyl. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like. In several embodiments, the phospholipid-polymer-aromatic conjugate may be represented by:

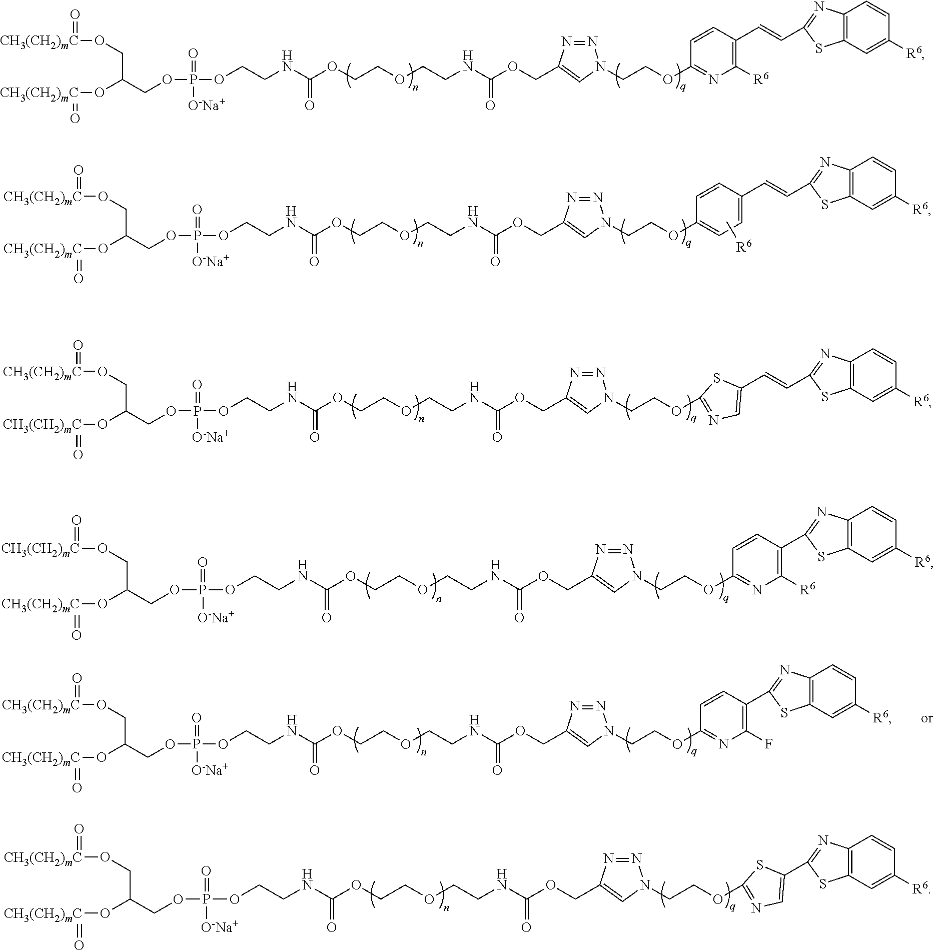

The variable n may be any integer from about 10 to about 100, for example, about 60 to about 100, about 70 to about 90, about 75 to about 85, about 77, and the like. The variable m may be one of: 12, 13, 14, 15, 16, 17, or 18. The variable q may be independently selected from a range of between about 1 and about 12, between about 1 and about 8, or between about 1 and about 4, e.g., 1, 2, 3, or 4. For example, n may be 77, q may be 4, and m may be 14. In another example, n may be 77, q may be 1, and m may be 16. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments, the phospholipid-polymer-aromatic conjugate may be represented by:

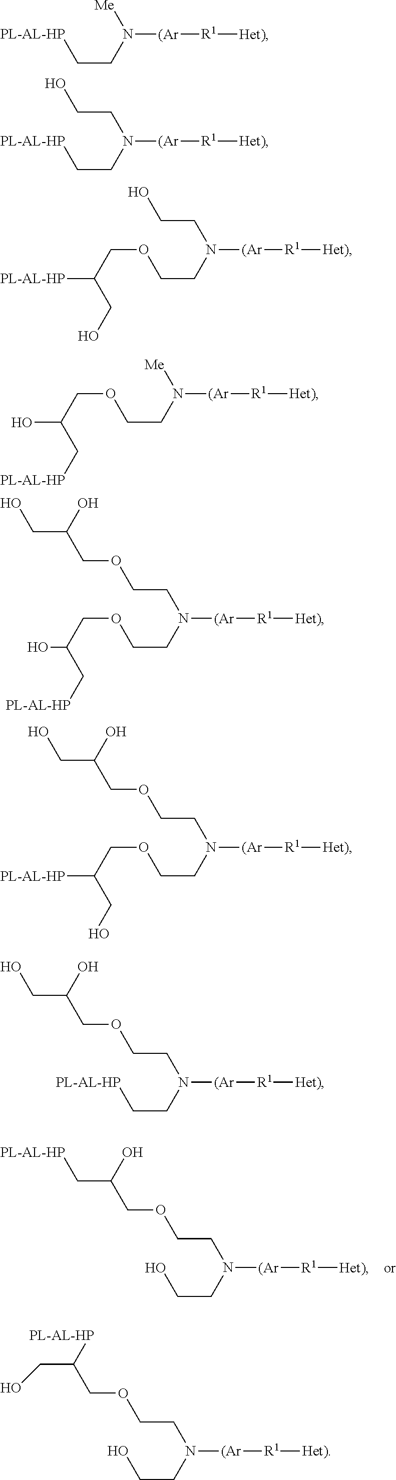

The group (Ar—R¹-Het) may be bonded to the rest of the phospholipid-polymer-aromatic conjugate by Ar or Het, e.g., by Ar.

In some embodiments of Structural Formula IV, the phospholipid-polymer-aromatic conjugate may be represented by:

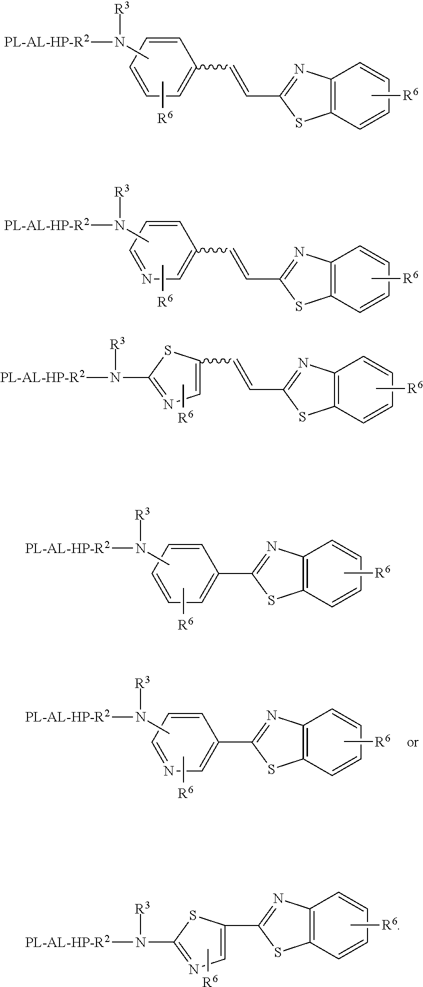

Similarly, the compound of Structural Formula V may be represented by:

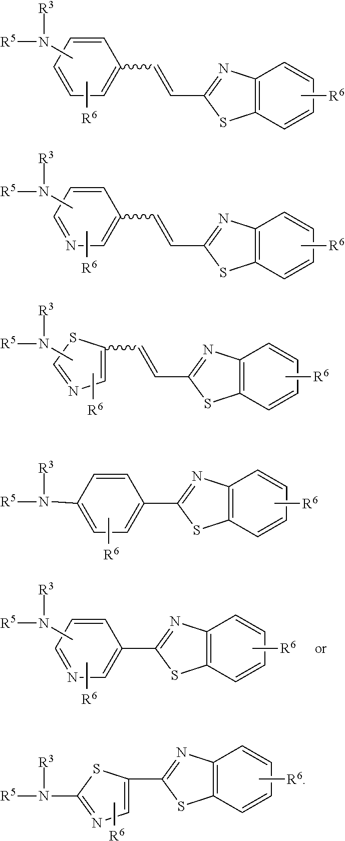

R⁵ may be H, hydroxyl, H—R²—, HO—R²—, H—R²—N(R³)—, or HO—R²—N(R³)—. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments of Structural Formula IV, the phospholipid-polymer-aromatic conjugate may be represented by:

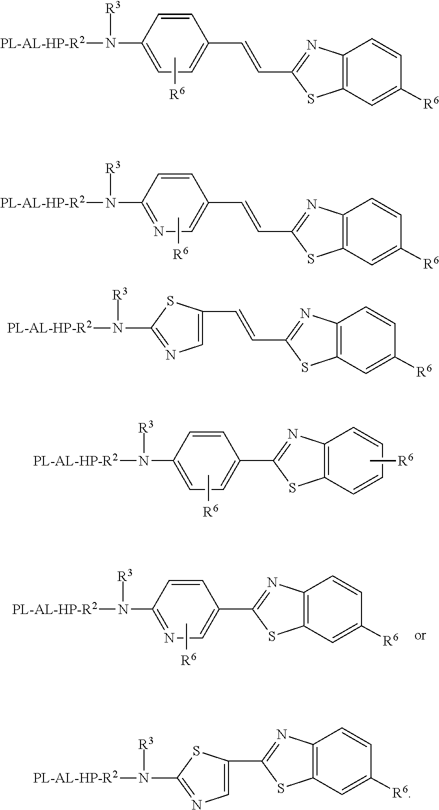

Similarly, the compound of Structural Formula V may be represented by:

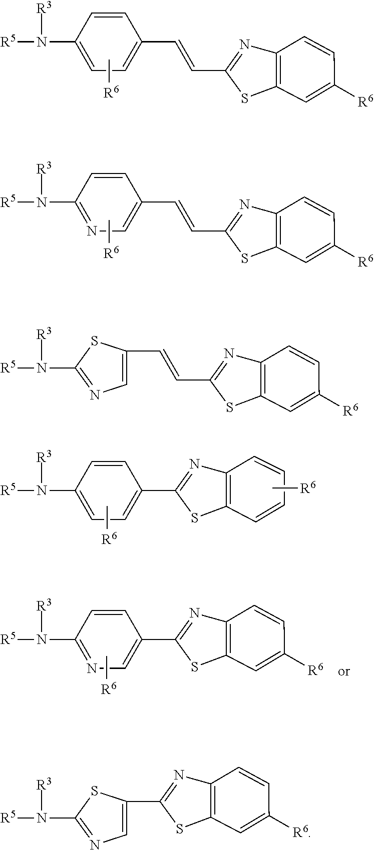

R⁵ may be H, hydroxyl, H—R²—, HO—R²—, H—R²—N(R³)—, or HO—R²—N(R³)—. Each R⁶ may independently be —H, —OH, alkyl, —O-alkyl, —F, or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In several embodiments, the phospholipid-polymer-aromatic conjugate may be represented by one of:

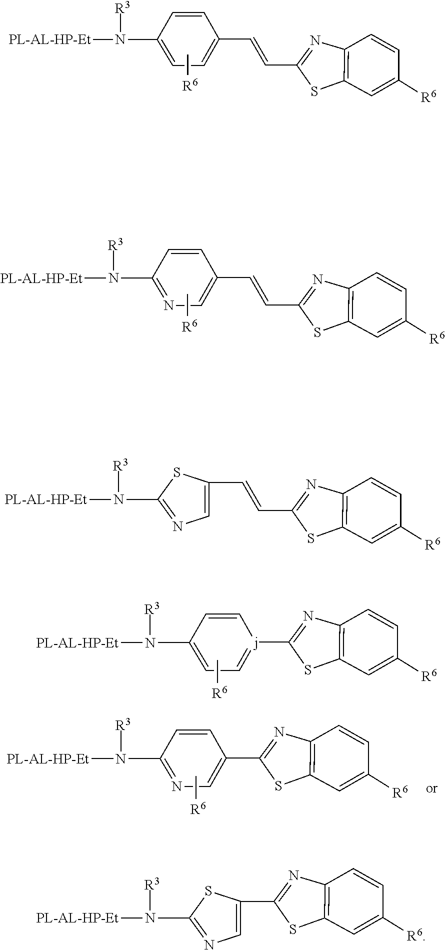

Each R⁶ may independently be —H, —OH, alkyl, —O-alkyl, —F, or —O-fluoroalkyl. Each R³ may independently be H, Me, or EtOH. Each amine, thiazole, and benzothiazole nitrogen may independently and optionally be alkylated to form a quaternary ammonium accompanied by a pharmaceutically acceptable anion, e.g., a halide ion, an acetate ion, and the like.

In various embodiments, the phospholipid-polymer-aromatic conjugate may be represented by one of:

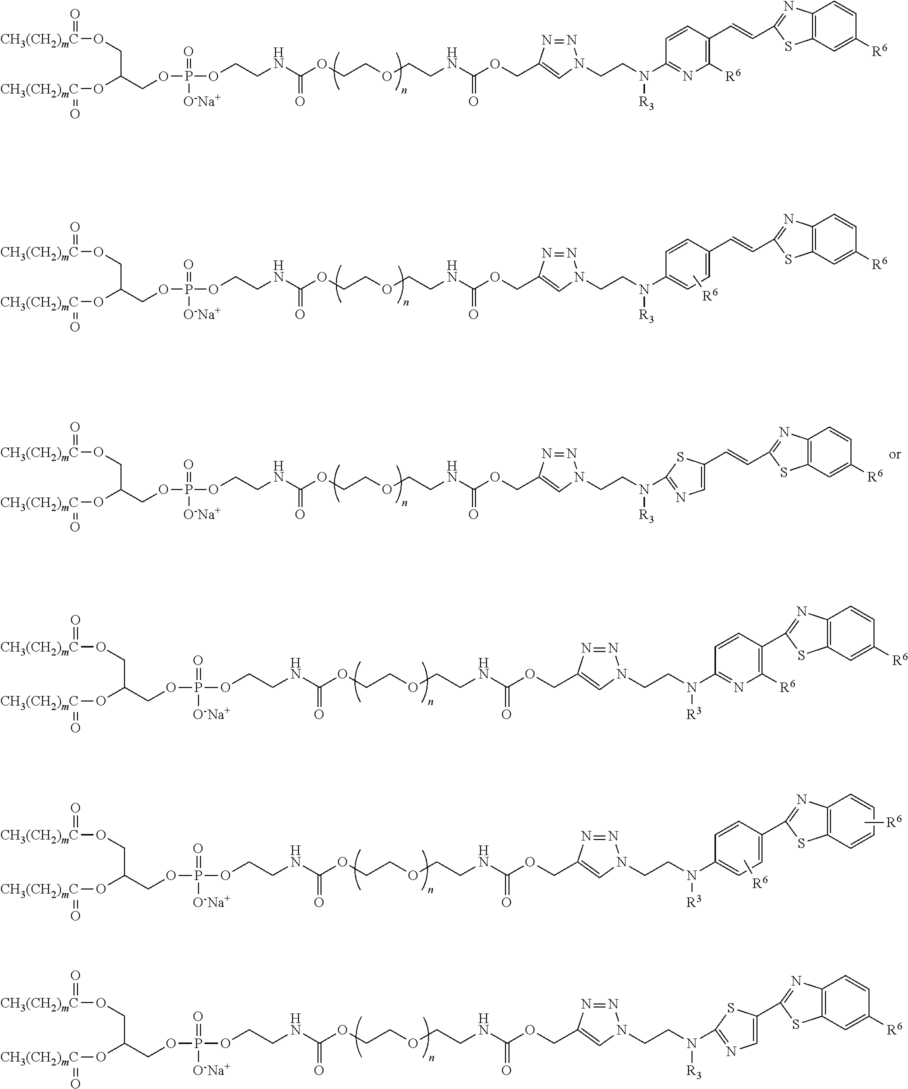

variable n may be any integer from about 10 to about 100, for example, about 60 to about 100, about 70 to about 90, about 75 to about 85, or about 77. The variable m may be one of: 12, 13, 14, 15, 16, 17, or 18. For example, n may be 77 and m may be 14. In another example, n may be 77 and m may be 16. Each R⁶ may independently be —H, —OH, alkyl, —O-alkyl, —F, or —O— fluoroalkyl. Each R³ may independently be H, Me, or EtOH. Each amine, thiazole, and benzothiazole nitrogen may independently and optionally be alkylated to form a quaternary ammonium accompanied by a pharmaceutically acceptable anion, e.g., a halide ion, an acetate ion, and the like. For example, the conjugate may be one of:

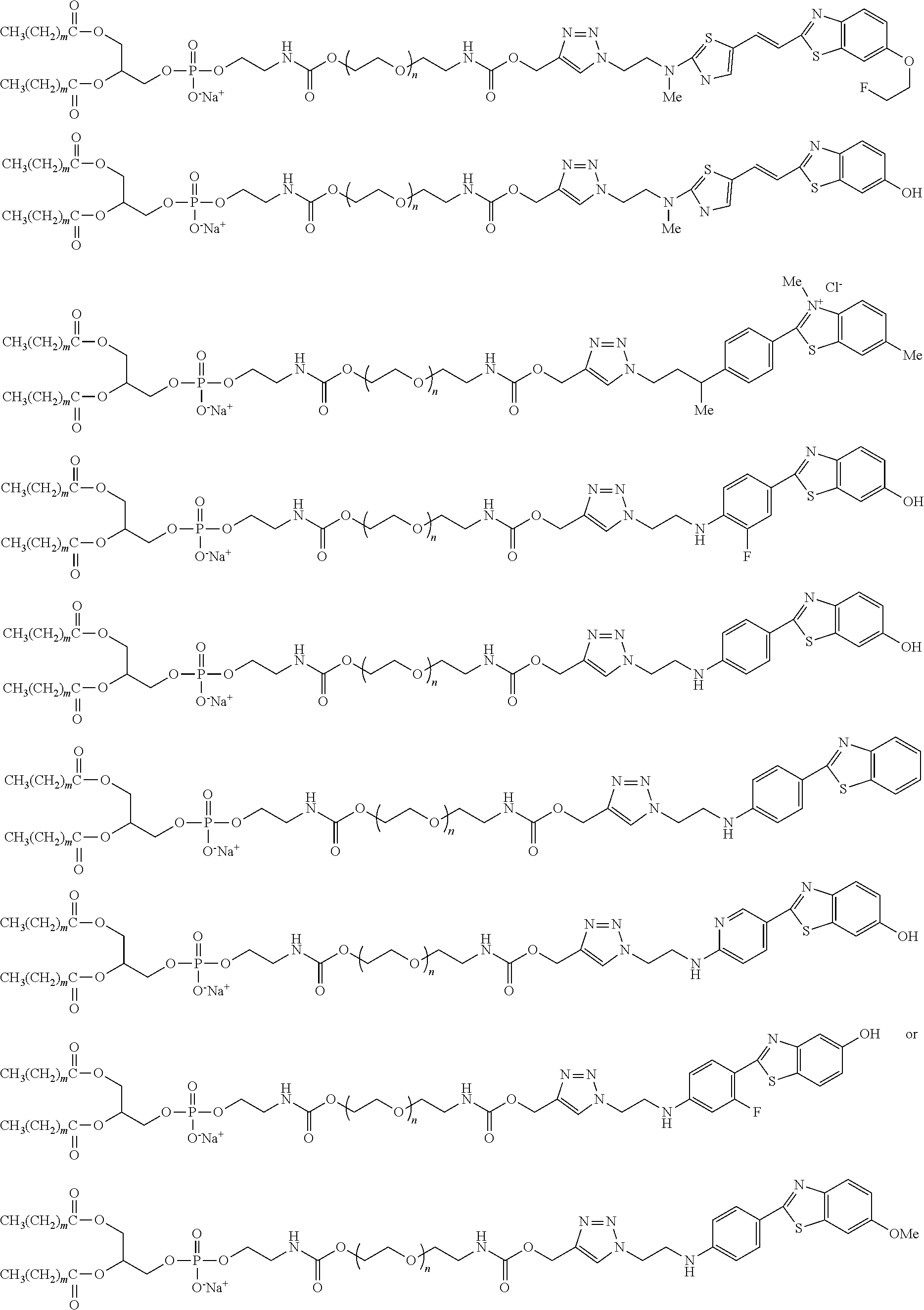

The invention also includes phospholipid-polymer-aromatic conjugates represented by Structural Formula VI:

PL-AL-HP-X—((Ar¹—R¹)ₚ—Ar²) (VI)

or a pharmaceutically acceptable alt thereof. PL may be a phospholipid. AL may be an aliphatic linkage. HP may be a hydrophilic polymer. X may be a bond, —O—, —R²—O—, —R²—O(C═O)—, —R²—N(R³)—O(C═O)—, —R²—N(R³)(C═O)—, or —R²—N(R³)—. R¹ may be C₂-C₆ alkyl or alkenyl. The variable p may be 0 or 1. R² may be a linking group including 1 to 6 carbon atoms. R² may include one of: alkylene or alkoxyalkylene. R³ may be hydrogen, C₁-C₆ alkyl, or C₁-C₆ alkoxyalkyl. Ar¹ may be a monocyclic or polycyclic group. Ar¹ may include at least one aromatic or heteroaromatic ring. Ar² may be a fused polycyclic aromatic hydrocarbon. X may be bonded to one of Ar¹ or Ar². The X, Ar¹, R¹, Ar², and variables therein such as R² and R³ may further be substituted. For example, R² may be substituted with zero, one or more of: hydroxyl, C₁-C₆ alkyl, and C₁-C₆ hydroxyalkyl. Ar¹, Ar², R¹, and R³ other than hydrogen may be independently substituted with 1, 2, or 3 of R⁶. Each R⁶ may be independently selected from —H; halogen; optionally alkylated methylenemalononitrile; —OH; —SH; alkyl; —O-alkyl; —S-alkyl; aryl; —O-aryl or —(O-alkylene)₁₋₆ optionally substituted with —OH or halogen; —NH₂; —NH— alkyl; —N-dialkyl; carboxyl; sulfonyl; carbamoyl; and glycosyl. In some embodiments, each R⁶ may independently be —H, —OH, alkyl, —O-alkyl, halogen; 1-(methyl)methylenemalononitrile; or —O-fluoroalkyl. The variables, e.g., X, Ar¹, R¹, Ar², and the like may represent the same moieties as in Structural Formula II described herein.

In some embodiments, a binding ligand represented by Structural Formula VII is provided:

R⁵—((Ar¹—R¹)ₚ—Ar²) (VII)

or a pharmaceutically acceptable salt thereof, wherein the variables, e.g., Ar¹, R¹, Ar², R⁵ and the like may represent the same moieties as in Structural Formula VI of the phospholipid-polymer-aromatic conjugate, or as in Structural Formula III of the binding ligand described herein.

In Structural Formula VII, R⁵ may be hydrogen, hydroxyl, H—R²—, HO—R²—, H—R²—N(R³)—, or HO—R²—N(R³)—. In some embodiments, R⁵ may be hydroxyl, H—R²—, HO—R²—, H—R²—N(R³)—, or HO—R²—N(R³)—. R⁵ may be H—R²—, HO—R²—, H—R²—N(R³)—, or HO—R²—N(R³)—. R⁵ may be H—R²— or H—R²—N(R³)—. R⁵ may be HO—R²— or HO—R²—N(R³)—. R⁵ may be H—R²— or HO—R²—.

In various embodiments of Structural Formulas VI and VII, R¹ may be C₂ alkyl or alkenyl. For example, R¹ may be C₂-C₆ alkenyl. R¹ may be C₂-C₆ alkenyl in a trans or cis configuration, for example, trans. R¹ may be trans 1,2-ethenyl.

In some embodiments of Structural Formulas VI and VII, one, two, three, or four ring atoms of the heteroaromatic rings included by Ar¹ each independently may be one of: N, O, or S. Ar¹ may include at least one heteroaromatic ring selected from the group consisting of: pyridine, pyrimidine, pyrazine, pyridazine, thiophene, furan, pyrrole, thiazole, oxazole, diazole, thiadiazole, oxadiazole, and triazole. Ar¹ may include, for example, one of: phenyl, pyridine, pyrimidine, pyrazine, pyridazine, thiophene, furan, pyrrole, thiazole, oxazole, diazole, thiadiazole, oxadiazole, triazole, benzofuran, indole, benzothiophene, thienopyrimidine, benzooxazole, benzothiazole, benzooxadiazole, or benzothiadiazole. Ar¹ may include one of phenyl or indole. Ar¹ may include phenyl, pyridine, or thiazole.

Ar² may be one of naphthalene, anthracene, phenanthrene, 1H-indene, 1H-cyclopenta[b]naphthalene, 9H-fluorene, 1H-cyclopenta[a]naphthalene, 1,5-dihydro-s-indacene, or 1,6-dihydro-as-indacene. Ar² may be one of naphthalene, anthracene, phenanthrene, 1H-indene, or 9H-fluorene. Ar² may be one of naphthalene and and 1H-indene. Ar² may be naphthalene.

In several embodiments of Structural Formulas VI and VII, Ar¹ and Ar² may be independently substituted with zero, one or more of: F, C₁, Br, I, 1-(alkyl)methylenemalononitrile, alkyl, aryl, —OH, —O-alkyl, —O-aryl, —NH₂, —NH-alkyl, —N— dialkyl, carboxyl, sulfonyl, carbamoyl, and glycosyl.

In various embodiments of Structural Formulas VI and VII, each amine and heteroaromatic ring nitrogen may independently and optionally be alkylated to form a quaternary ammonium accompanied by a pharmaceutically acceptable anion, e.g., a halide ion, an acetate ion, and the like. For example, each amine, thiazole, and benzothiazole nitrogen may independently and optionally be alkylated to form a quaternary ammonium accompanied by a pharmaceutically acceptable anion, e.g., a halide ion, an acetate ion, and the like.

In various embodiments of Structural Formulas VI and VII, the phospholipid-polymer-aromatic conjugate may be represented by PL-AL-HP—O—((Ar¹—R¹)ₚ-Ar²). The compound may be represented by H—O—((Ar¹—R¹)ₚ-Ar²). Ar² and/or Ar¹ may be substituted by —O-alkyl. Ar² and/or Ar¹ may be substituted by methoxy.

In some embodiments of Structural Formula VI, the phospholipid-polymer-aromatic conjugate may be represented by:

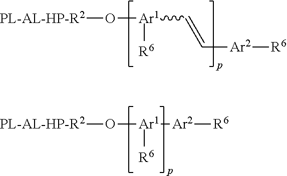

Similarly, the binding ligand of Structural Formula VII may be represented by:

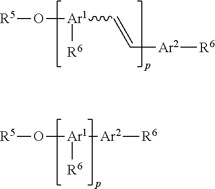

R⁵ may be H, hydroxyl, H—R²—, HO—R²—, H—R²—N(R³)—, or HO—R²—N(R³)—. The variable p may be 0 or 1. Each R⁶ may independently be —H, —OH, alkyl, —O-alkyl, halogen; 1-(methyl)methylenemalononitrile; or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments, the phospholipid-polymer-aromatic conjugate may be represented by:

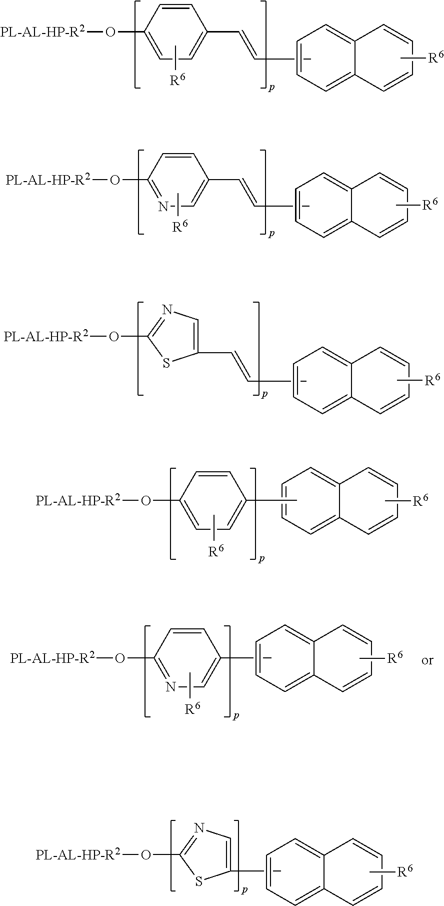

Similarly, the compound of Structural Formula VII may be represented by:

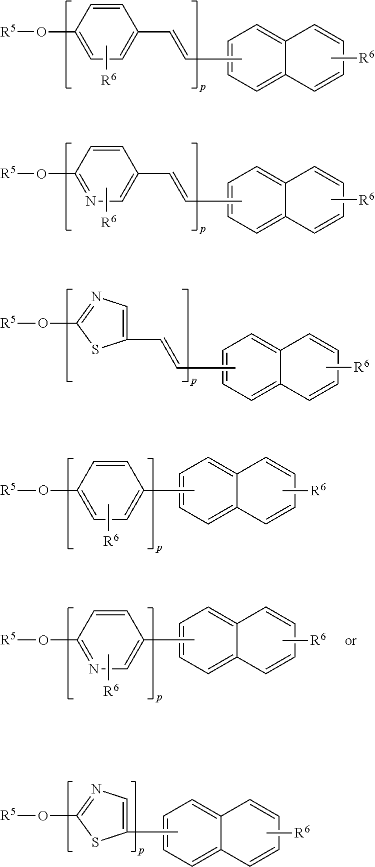

R⁵ may be H, hydroxyl, H—R²—, HO—R²—, H—R²—N(R³)—, or HO—R²—N(R³)—. In some embodiments, R⁵ may be hydroxyl. The variable p may be 0 or 1. Each R⁶ may independently be —H, —OH, alkyl, —O-alkyl, halogen; 1-(methyl)methylenemalononitrile; or —O— fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like. In several embodiments, the phospholipid-polymer-aromatic conjugate may be represented by:

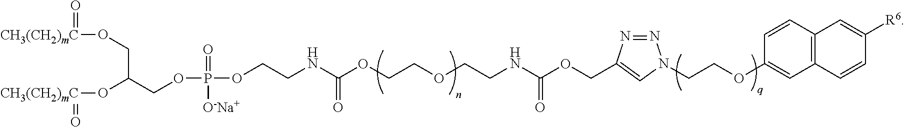

The variable n may be any integer from about 10 to about 100, for example, about 60 to about 100, about 70 to about 90, about 75 to about 85, about 77, and the like. The variable m may be one of: 12, 13, 14, 15, 16, 17, or 18. The variable q may be independently selected from a range of between about 1 and about 12, between about 1 and about 8, or between about 1 and about 4, e.g., 1, 2, 3, or 4. For example, n may be 77, q may be 4, and m may be 14. In another example, n may be 77, q may be 1, and m may be 16. The variable p may be 0 or 1. Each R⁶ may independently be —H, —OH, alkyl, —O-alkyl, halogen; 1-(methyl)methylenemalononitrile; or —O— fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments, the phospholipid-polymer-aromatic conjugate may be represented by:

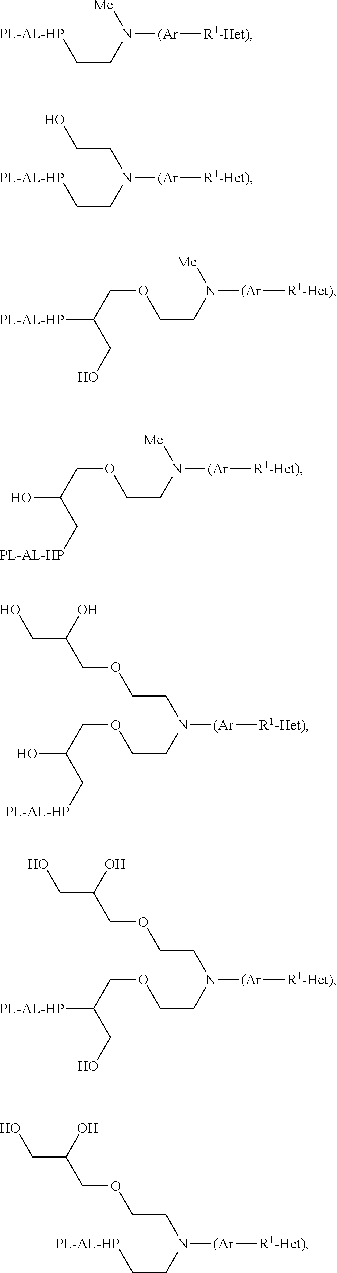

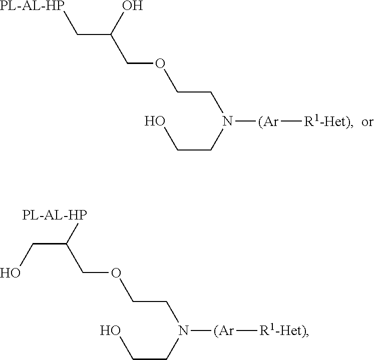

The group ((Ar¹—R¹)ₚ-Ar²) may be bonded to the rest of the phospholipid-polymer-aromatic conjugate by Ar¹ or Ar², e.g., by Ar¹.

In some embodiments of Structural Formula VI, the phospholipid-polymer-aromatic conjugate may be represented by:

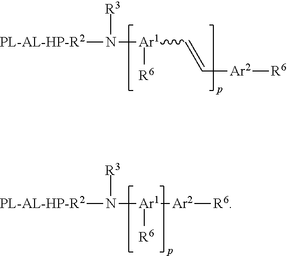

Similarly, the binding ligand of Structural Formula VII may be represented by:

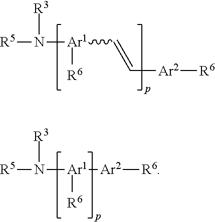

R⁵ may be H, hydroxyl, H—R²—, HO—R²—, H—R²—N(R³)—, or HO—R²—N(R³)—. The variable p may be 0 or 1. Each R⁶ may independently be —H, —OH, alkyl, —O-alkyl, halogen; 1-(methyl)methylenemalononitrile; or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In some embodiments of Structural Formula VI, the phospholipid-polymer-aromatic conjugate may be represented by:

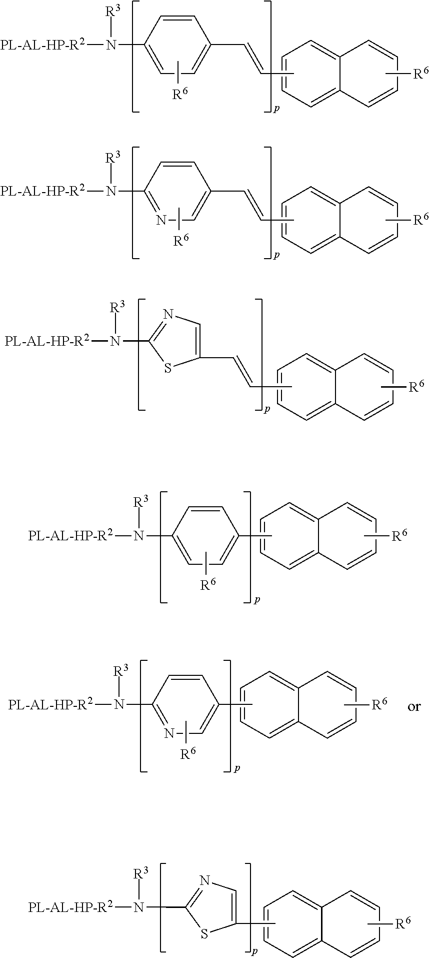

Similarly, the binding ligand of Structural Formula VII may be represented by:

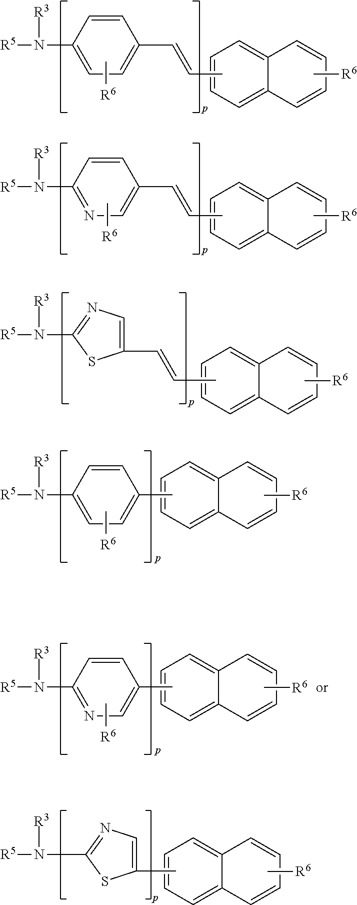

R⁵ may be H, hydroxyl, H—R²—, HO—R²—, H—R²—N(R³)—, or HO—R²—N(R³)—. The variable p may be 0 or 1. Each R⁶ may independently be —H, —OH, alkyl, —O-alkyl, halogen; 1-(methyl)methylenemalononitrile; or —O-fluoroalkyl. Each amine, thiazole, and benzothiazole nitrogen may independently and optionally be alkylated to form a quaternary ammonium accompanied by a pharmaceutically acceptable anion, e.g., a halide ion, an acetate ion, and the like.

In several embodiments, the phospholipid-polymer-aromatic conjugate may be represented by one of:

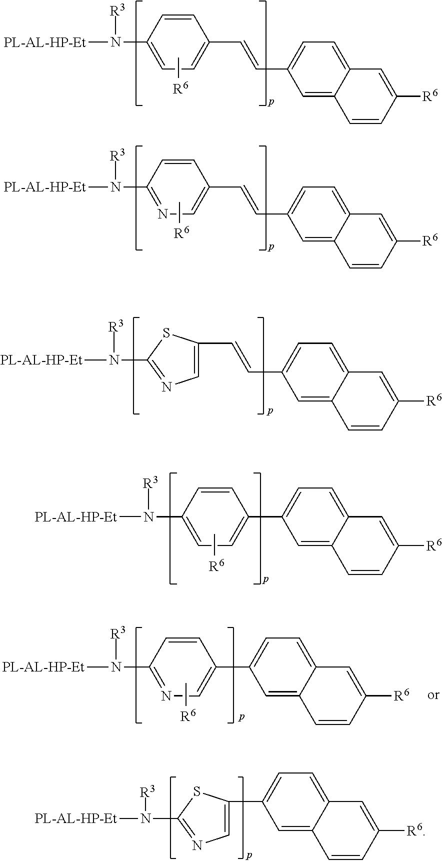

Each R⁶ may independently be —H, —OH, alkyl, —O-alkyl, halogen; 1-(methyl)methylenemalononitrile; or —O-fluoroalkyl. The variable p may be 0 or 1. Each R³ may independently be H, Me, or EtOH. Each amine, thiazole, and benzothiazole nitrogen may independently and optionally be alkylated to form a quaternary ammonium accompanied by a pharmaceutically acceptable anion, e.g., a halide ion, an acetate ion, and the like.

In various embodiments, the phospholipid-polymer-aromatic conjugate may be represented by one of:

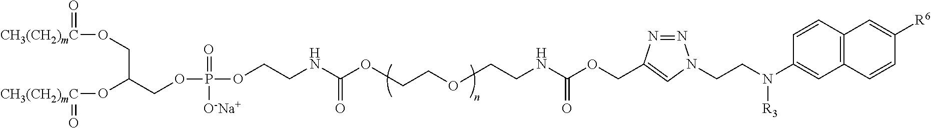

The variable n may be any integer from about 10 to about 100, for example, about 60 to about 100, about 70 to about 90, about 75 to about 85, or about 77. The variable m may be one of: 12, 13, 14, 15, 16, 17, or 18. For example, n may be 77 and m may be 14. In another example, n may be 77 and m may be 16. The variable p may be 0 or 1. Each R⁶ may independently be —H, —OH, alkyl, —O-alkyl, halogen; 1-(methyl)methylenemalononitrile; or —O-fluoroalkyl. Each R³ may independently be H, Me, or EtOH. Each amine, thiazole, and benzothiazole nitrogen may independently and optionally be alkylated to form a quaternary ammonium accompanied by a pharmaceutically acceptable anion, e.g., a halide ion, an acetate ion, and the like. For example, the conjugate may be one of:

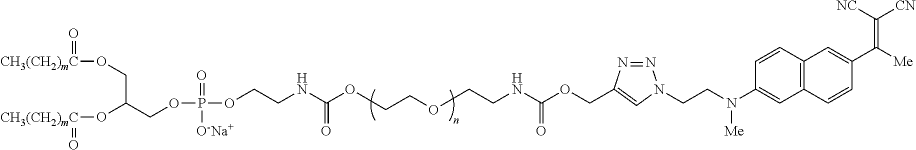

### Polymers and Phospholipids

The phospholipid-polymer aromatic conjugate includes a phospholipid-polymer region that facilitates incorporation of the conjugate into a membrane such as that present in a liposome. In some embodiments, the phospholipid moiety PL in the phospholipid-polymer-aromatic conjugate may be represented by the following structural formula:

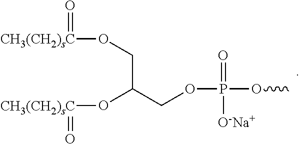

The variable s may be one of: 12, 13, 14, 15, 16, 17, or 18. For example, s may be 14 or 16. In various embodiments, the phospholipid moiety in the phospholipid-polymer-aromatic conjugate may be one of: 1,2-dipalmitoyl-sn-glycero-3-phosphocholine (DPPC), 1,2-distearoyl-sn-glycero-3-phosphoethanolamine (DSPE), 1,2-distearoyl-sn-glycero-3-phosphocholine (DSPC), or 1,2-Dipalmitoyl-sn-glycero-3-phospho ethanolamine (DPPE). Suitable phospholipids may also include those disclosed herein, and may further include those disclosed in U.S. Pat. No. 7,785,568 issued to Annapragada et al., which is incorporated by reference herein in its entirety. Suitable polymer derivatized phospholipids may include those disclosed herein, and may further include those disclosed in U.S. Pat. No. 7,785,568, the entire contents of which are incorporated herein by reference.

In some embodiments, the polymer moiety in the phospholipid-polymer-aromatic conjugate may include a hydrophilic polymer, e.g., a poly(alkylene oxide) polymer. The hydrophilic poly(alkylene oxide) may include between about 10 and about 100 repeat units, and may have, e.g., a molecular weight ranging from 500-10,000 Daltons. The hydrophilic poly(alkylene oxide) may include, for example, poly(ethylene oxide) (“PEG”), poly (propylene oxide) (“PPO”), and the like. The hydrophilic polymer HP may be conjugated to the phospholipid moiety via an amide or carbamate group, as described herein. The polymer moiety in the phospholipid-polymer-aromatic conjugate may be conjugated to the aromatic moiety via an amide, carbamate, poly (alkylene oxide), triazole, combinations thereof, and the like. For example, the polymer moiety in the phospholipid-polymer-aromatic conjugate may be represented by one of the following structural formula:

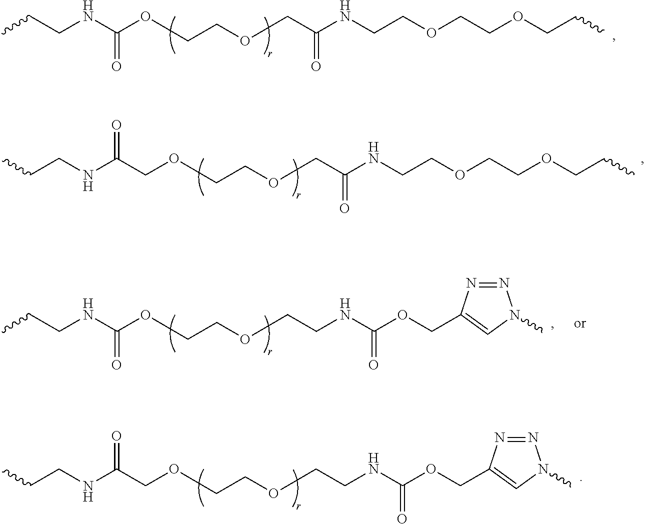

The variable r may be independently selected in a range of between about 10 to about 100, between about 60 to about 100, between about 70 to about 90, between about 75 to about 85, or about 77.

In several embodiments, the phospholipid-polymer moiety PL-HP— in the phospholipid-polymer-aromatic conjugate may be represented by one of the following structural formula:

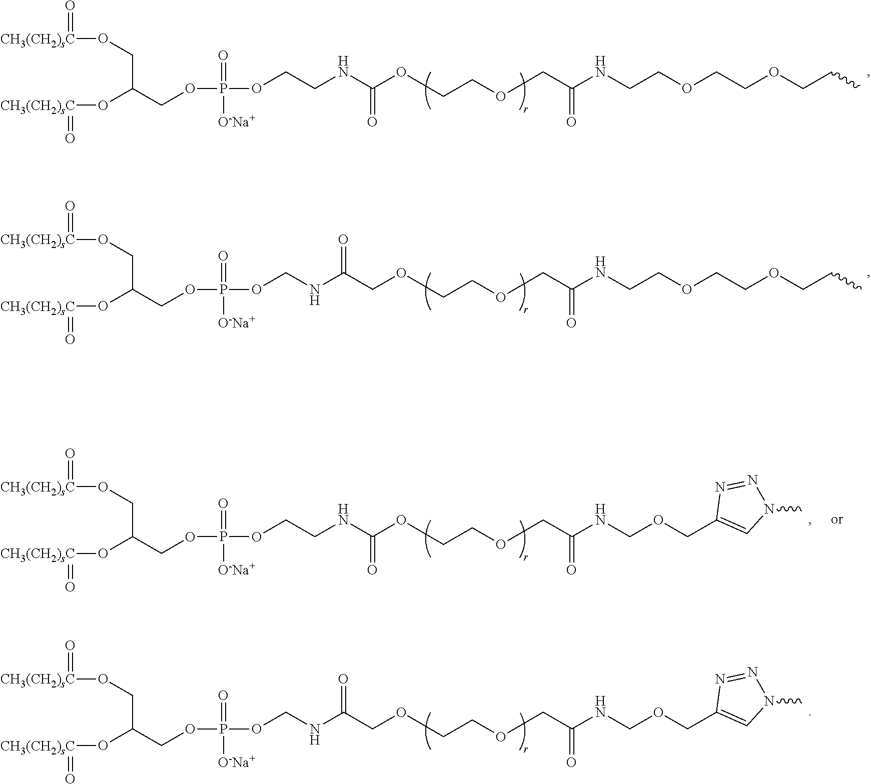

The variable r may be independently selected in a range of between about 10 to about 100, between about 60 to about 100, between about 70 to about 90, between about 75 to about 85, or about 77. The variable s may be independently selected in a range of between about 12 and about 18, or one of: 12, 13, 14, 15, 16, 17, or 18, or one of 12, 14, 16, or 18, or 14 or 16. For example, r may be about 77 and s may be 14. In another example, r may be about 77 and s may be 16.

In some embodiments, q is a range of repeat units of between about one of: 1 and 12, 1 and 8, or 1 and 4; r is a range of repeat units of between about one of: 10 and 100, 60 and 100, 70 and 90, or 75 and 85; and s is one of: 12, 13, 14, 15, 16, 17, or 18. In some embodiments, q is from 1 to 8; r is between 70 and 90; and s is one of: 12, 14, 16, or 18. In some embodiments, q is from 1 to 4; r is between about 70 and 90; and s is one of: 12, 14, 16, or 18. In some embodiments, q is from 1 to 4; r is between about 75 and 85, e.g., 77; and s is one of: 14 or 16. In several embodiments, q is about 4; r is about 77; and s is about 14. For example, the conjugate may include Conjugate A or Conjugate A′ in FIG. 3.

As used herein, an “aliphatic linkage” represented by AL includes any aliphatic group useful for linking between a phospholipid PL and a hydrophilic polymer HP. Such aliphatic linkages may include, for example, C₂-C₁₀ alkylene groups, which may include heteroatoms via one or more moieties such as amides, carbamates, and the like. For example, in the conjugate below:

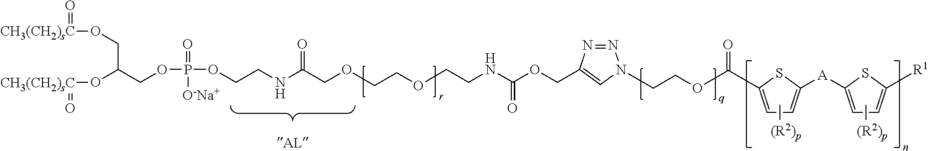

the aliphatic linkage AL, —CH₂CH₂NH(C═O)CH₂O—, includes an amide moiety. Further, for example, in the conjugate below:

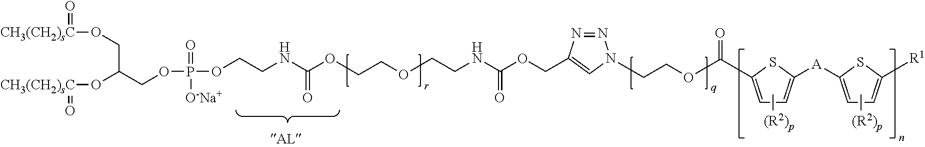

the aliphatic linkage AL, —CH₂CH₂NH(C═O)O—, includes a carbamate moiety. AL may include aliphatic linkages derived from dicarboxylic acids, such as succinic acid, and may include two amides, two carbamates, an amide and a carbamate, and the like.

Such aliphatic linkages are known in the art for linking between a phospholipid and a hydrophilic polymer, and may be found, for example, in commercial sources of phospholipid-PEG compounds, and functionalized phospholipid-PEG conjugation precursors, which may be represented as PL-AL-PEG-NH₂, PL-AL-PEG-CO₂H, and the like. It should be noted that it is common in the art and in commercial sources to refer to such compounds in abbreviated form without reference to the aliphatic linkage, where the presence of the aliphatic linkage is implied. For example, 1,2-distearoyl-sn-glycero-3-phosphoethanolamine-N-[methoxy(polyethylene glycol)-2000] CAS No. 147867-65-0, in which the aliphatic linking group is the amide containing group —CH₂CH₂NH(C═O)CH₂O—, is commonly referred to in the art and commercially as “DSPE-mPEG-2000.” Commercial materials recited herein in the conventional abbreviated manner, such as “DSPE-mPEG-2000,” should be understood to include corresponding aliphatic linkages.

Further, it has now been now found for such compounds that among various commercial sources and even different lots from the same commercial source may contain a mixture of compounds with different aliphatic linkers, e.g., a mixture of compounds having amine and carbamate aliphatic linkers. The results described in the Examples were found to be similar using various different conjugates with AL including carbamate, amide, and mixtures thereof.

Accordingly, in various embodiments, the aliphatic linker represented by AL may include a carbamate or an amide. The liposomes, methods, and conjugates described herein may include phospholipid-polymer-aromatic compound conjugates wherein AL includes a carbamate, an amide, or a mixture of such conjugates.

### Liposomal Compositions

In various embodiments, a liposomal composition is provided. A liposome, as is known by those skilled in the art, is a roughly spherical vesicle comprising at least one lipid bilayer which forms a membrane that surrounds a generally aqueous core. The membrane may include the phospholipid-polymer-aromatic conjugate or a pharmaceutically acceptable salt thereof according to any of the embodiments described herein.

In some embodiments, the membrane of a liposomal composition may include the phospholipid-polymer-aromatic conjugate represented by Structural Formula I. In further embodiments, the liposomal composition can include a phospholipid-polymer conjugate according to Structural formula II, IV, or VI. In some embodiments, the liposomal composition includes a phospholipid-polymer conjugate according to Structural Formula II.

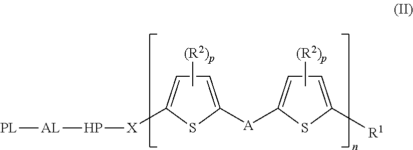

or a pharmaceutically acceptable salt thereof.

In embodiments including a liposome, an imaging agent may also be included. The imaging agent may be selected from imaging agents detectable with a suitable technique for in vivo imaging, such as PET, SPECT, NMR, MRS, MRI, and CAT. For example, the imaging agent may be a nonradioactive magnetic resonance imaging (MRI) contrast enhancing agent. The imaging agent may be at least one of encapsulated by or bound to the membrane. For example, the nonradioactive magnetic resonance imaging (MRI) contrast enhancing agent may be both encapsulated by and bound to the membrane, e.g., to provide a dual contrast agent liposome. The liposomal composition may be characterized by a per-particle relaxivity in mM⁻¹s⁻¹ of at least about one or more of about: 100,000, 125,000, 150,000, 165,000, 180,000, 190,000, and 200,000. Detecting the liposomal formulation may include detecting using magnetic resonance imaging, for example, in a magnetic field range of between about 1 T to about 3.5 T, or about 1.5 to about 3 T. The nonradioactive MRI contrast enhancing agent may include gadolinium. For example, the nonradioactive MRI contrast enhancing agent may include (diethylenetriaminepentaacetic acid)-bis(stearylamide), gadolinium salt (Gd-DTPA-BSA). Gadolinium paramagnetic chelates such as GdDTPA, GdDOTA, GdHPDO3A, GdDTPA-BMA, and GdDTPA-BSA are known MRI contrast agents. See U.S. Pat. No. 5,676,928 issued to Klaveness et al., which is incorporated by reference herein in its entirety.

In several embodiments, the liposomal composition may include a radioactive contrast enhancing agent that is at least one of encapsulated by or bound to the membrane. The radioactive contrast enhancing agent may include, for example, those agents deemed appropriate for use with SPECT imaging and/or PET imaging in the National Institute of Health's Molecular Imaging and Contrast Agent Database (“MICAD”).

In some embodiments, the membrane may include one or more stabilizing excipients. The one or more stabilizing excipients may include a sterol, e.g., cholesterol, or a fatty acid.

In several embodiments, the membrane may include a first phospholipid. The membrane may include a second phospholipid. The second phospholipid may be derivatized with a hydrophilic polymer that may include, for example, a hydrophilic poly(alkylene oxide). The hydrophilic poly(alkylene oxide) may include between about 10 and about 100 repeat units. The hydrophilic poly(alkylene oxide) may include, for example, poly(ethylene oxide), poly (propylene oxide) and the like. As used herein, the phospholipid moieties in each of the “first phospholipid,” the “second phospholipid,” and in the phospholipid-polymer-aromatic conjugate may be selected independently.

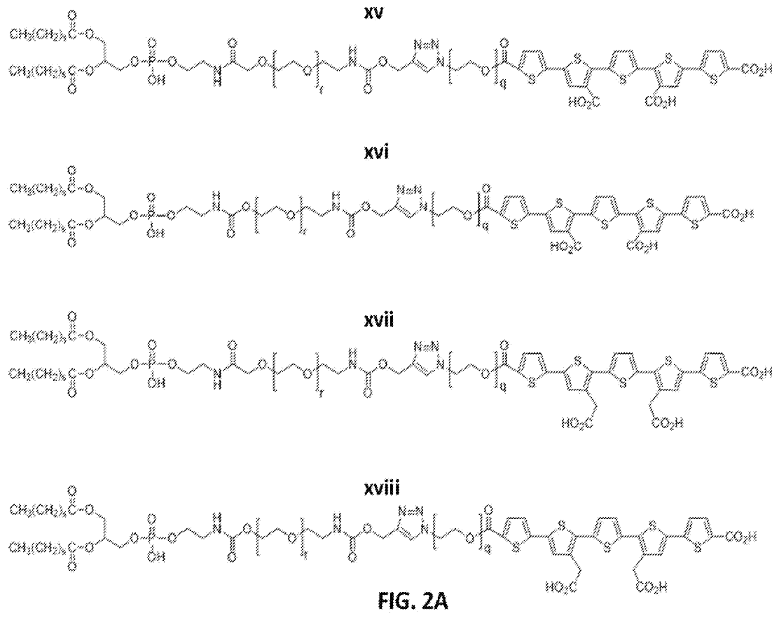

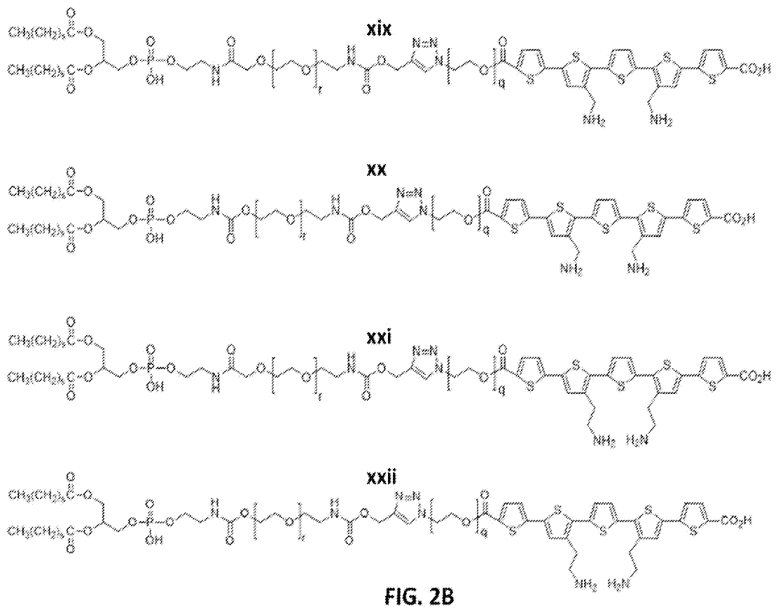

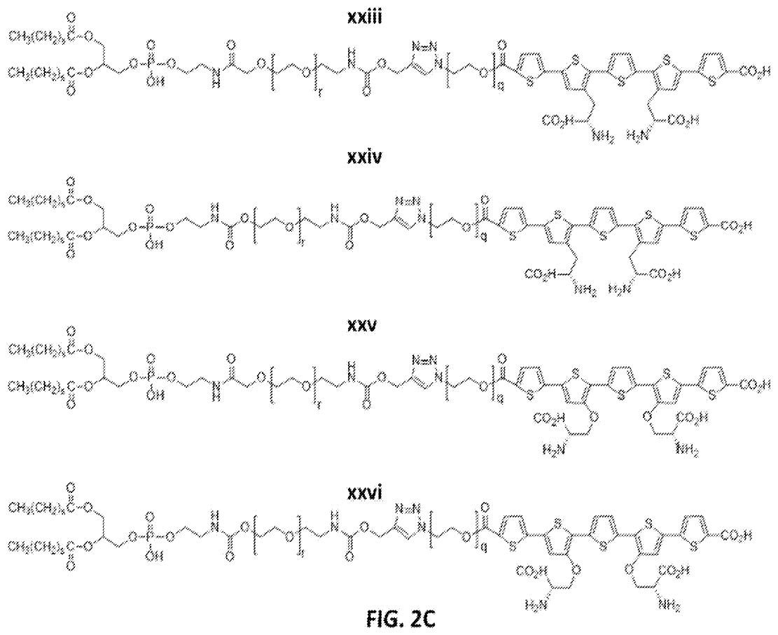

In various embodiments, the membrane of the liposome composition may include: DPPC; cholesterol; diethylenetriamine pentaacetic acid)-bis(stearylamide), gadolinium salt; and 1,2-distearoyl-sn-glycero-3-phosphoethanolamine-N— [methoxy (polyethylene glycol)-2000](“DSPE-mPEG-2000”; CAS No. 147867-65-0). The phospholipid-polymer-aromatic conjugate may be represented by one of Structural Formulas xv-xxvi in FIGS. 2A, 2B, and 2C, or a pharmaceutically acceptable salt thereof. The variable q may be a range of repeat units of between about one of: 1 and 12, 1 and 8, or 1 and 4; r may be in a range of repeat units of between about one of: 10 and 100, 60 and 100, 70 and 90, or 75 and 85; and s may be one of: 12, 13, 14, 15, 16, 17, or 18. In some embodiments, q may be from 1 to 8; r may be between 70 and 90; and s may be one of: 12, 14, 16, or 18. In some embodiments, q may be from 1 to 4; r may be between about 75 and 85; and s may be one of: 14 or 16.

In various embodiments, the membrane of the liposome composition may include: DPPC; cholesterol; diethylenetriamine pentaacetic acid)-bis(stearylamide), gadolinium salt; and 1,2-distearoyl-sn-glycero-3-phosphoethanolamine-N— [methoxy (polyethylene glycol)-2000](“DSPE-mPEG-2000”; CAS No. 147867-65-0). The phospholipid-polymer-aromatic conjugate may be represented by one or both of Conjugate A or Conjugate A′ in FIG. 3, e.g., Conjugate A, or a pharmaceutically acceptable salt thereof.

### Methods of Imaging and Diagnosis

Another aspect of the invention provides a method for imaging one or more misfolded and/or aggregated proteins in a subject. The method may include introducing into the subject a detectable quantity of the liposomal composition. The method may include allowing sufficient time for the liposomal composition to be associated with the one or more misfolded and/or aggregated proteins. The method may include detecting the liposomal composition associated with the one or more misfolded and/or aggregated proteins. The membrane of the liposome includes a phospholipid-polymer-aromatic conjugate represented by Structural Formula I, II, IV, or VI, or a pharmaceutically acceptable salt thereof.

In some embodiments, the detecting may include detecting using magnetic resonance imaging. In another example, the detecting may include detecting by fluorescence imaging (FI). The detecting may include detecting by SPECT imaging and/or PET imaging, and the non-radioactive contrast enhancing agent may be replaced with a radioactive contrast enhancing agent. The radioactive contrast enhancing agent may include, for example, those agents deemed appropriate for use with SPECT imaging and/or PET imaging in the National Institute of Health's Molecular Imaging and Contrast Agent Database (“MICAD”). Any other suitable type of imaging methodology known by those skilled in the art is contemplated, including, but not limited to, PET imaging.

In several embodiments, the one or more misfolded proteins may include one or more of: prion protein, beta-amyloid (Aβ), α-synuclein (αS), and tau. For example, the one or more misfolded proteins may include prion protein. The one or more misfolded proteins may include Aβ protein. The one or more misfolded proteins may include Aβ and tau protein. The one or more misfolded proteins may include αS protein. The one or more misfolded proteins may include αS and tau protein.

In various embodiments, the method may include diagnosing the subject with Alzheimer's disease according to detecting the liposomal composition associated with the one or more misfolded proteins comprising one or both of Aβ and tau. The method may include diagnosing the subject with Parkinson's disease according to detecting the liposomal composition associated with the one or more misfolded proteins comprising one or both of αS and tau. The method may include diagnosing the subject with a prion disease according to detecting the liposomal composition associated with the one or more misfolded proteins comprising prion protein.

In some embodiments, the method may include identifying the subject as potentially having Alzheimer's disease according to detecting the liposomal composition associated with the one or more misfolded proteins including one or more amyloid deposits. The method may include subjecting the subject to an analysis for tau, e.g., using the disclosed liposome, or analyzing for neurofibrillary tangles using, for example, a PET analysis for tau neurofibrillary tangles. The method may include diagnosing the patent with Alzheimer's disease upon determining the presence of misfolded tau or tau neurofibrillary tangles in conjunction with detecting the liposomal composition associated with the one or more amyloid deposits.

### Imaging Kits

Another aspect of the invention provides a kit for imaging one or more misfolded and/or aggregated proteins in a subject. The kit may include instructions and the liposomal composition. The instructions may direct a user to introduce into the subject a detectable quantity of the liposomal composition. The instructions may direct the user to allow sufficient time for the liposomal composition to be associated with the one or more misfolded and/or aggregated protein. The instructions may direct the user to detect the liposomal composition associated with the one or more misfolded and/or aggregated proteins. The membrane of the liposome may include the phospholipid-polymer-aromatic conjugate represented by Structural Formula I, II, IV, or VI.

The kit can also include instructions for using the kit to carry out a method of detecting one or more misfolded proteins. In various embodiments, the instructions may direct a user to carry out any of the method steps described herein. For example, the instructions may direct a user to diagnose the patient with Alzheimer's disease according to detecting the liposomal composition associated with the one or more amyloid deposits. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

Components of the kits may be in different physical states. For example, some components may be lyophilized and some in aqueous solution. Some may be frozen. Individual components may be separately packaged within the kit. Other useful tools for performing the methods of the invention or associated testing, therapy, or calibration may also be included in the kits, including buffers, enzymes, fluorescent reagents, enhancing agents (e.g. paramagnetic ions) for magnetic resonance imaging (MRI), gels, plates, detectable labels, vessels, etc. Kits may also include a sampling device for obtaining a biological sample from a subject, such as a syringe or needle.

Various embodiments of the liposomal composition, the method, the phospholipid-polymer-aromatic conjugate for use in the method, the liposomal composition for use in the method, and the kits may employ the phospholipid-polymer-aromatic conjugate represented by Structural Formula I, and accordingly, each such embodiment explicitly contemplates each variable and value for Structural Formula I, as described herein. Moreover, in various embodiments of the binding ligand represented by Structural Formula I, each variable and value can include those described for more detailed Structural Formula, such as Structural Formula II and III.

For example, in various embodiments of Structural Formulas II and III, A may be C₂ alkyl or alkenyl. For example, A may be C₂-C₆ alkenyl. A may be C₂-C₆ alkenyl in a trans or cis configuration, for example, trans. A may be trans 1,2-ethenyl. A may be one of: thienylene, vinylene-thienylene, thienylene-vinylene, or vinylene-thienylene-vinylene, substituted with 0, 1, or 2 of R³.

### Definitions

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When “only A or B but not both” is intended, then the term “only A or B but not both” will be employed. Thus, use of the term “or” herein is the inclusive, and not the exclusive use. As used in the specification and the claims, the singular forms “a,” “an,” and “the” include the plural. Finally, where the term “about” is used in conjunction with a number, it is intended to include ±10% of the number. For example, “about 10” may mean from 9 to 11.

As used herein, when present as a terminal element of a chemical structure, the wavy bond symbol “ [symbol] ” indicates the position of attachment of the depicted structure to another described or depicted structure, for example, between an aromatic moiety represented by Structural Formula I to the remainder of the phospholipid-polymer-aromatic conjugate. When present as a connecting element within a chemical structure or connecting a defined group to a chemical structure, the wavy bond symbol “ [symbol] ” indicates a bond that encompasses all possible stereoisomeric or configurational possibilities. For example, in corresponding context, “ [symbol] ” may indicate a cis or trans configuration at a double bond, an R or S configuration at a stereocenter, and the like.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above and include, without limitation, haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments, the number of ring carbon atoms ranges from 3 to 5, 3 to 6, or 3 to 7. Bi— and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclic groups include aromatic (also referred to as heteroaryl) and non-aromatic ring compounds containing 3 or more ring members of which one or more is a heteroatom such as, but not limited to, N, O, and S. In some embodiments, the heterocyclyl group contains 1, 2, 3 or 4 heteroatoms. In some embodiments, heterocyclic groups include mono-, bi- and tricyclic rings having 3 to 16 ring members, whereas other such groups have 3 to 6, 3 to 10, 3 to 12, or 3 to 14 ring members. Heterocyclic groups encompass aromatic, partially unsaturated and saturated ring systems, such as, for example, imidazolyl, imidazolinyl and imidazolidinyl groups. The phrase “heterocyclic group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclic groups that have other groups, such as alkyl, oxo or halo groups, bonded to one of the ring members. Rather, these are referred to as “substituted heterocyclic groups.” Heterocyclic groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclic groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the technology are designated by use of the suffix, “ene.” For example, divalent alkyl groups are alkylene groups, divalent aryl groups are arylene groups, divalent heteroaryl groups are heteroarylene groups, and so forth. Substituted groups having a single point of attachment to the compound of the technology are not referred to using the “ene” designation. Thus, for example, chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include, but are not limited to, methoxy, ethoxy, propoxy, butoxy, pentoxy, hexoxy, and the like. Examples of branched alkoxy groups include, but are not limited to, isopropoxy, sec-butoxy, tert-butoxy, isopentoxy, isohexoxy, and the like. Examples of cycloalkoxy groups include, but are not limited to, cyclopropyloxy, cyclobutyloxy, cyclopentyloxy, cyclohexyloxy, and the like. Representative substituted alkoxy groups may be substituted one or more times with substituents such as those listed above.

The term “amine” (or “amino”), as used herein, refers to NR^{a}R^{b} groups, wherein R^{a} and R^{b} are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. The term “alkylamino” is defined as NR^{c}R^{d}, wherein at least one of R^{c} and R^{d} is alkyl and the other is alkyl or hydrogen. The term “arylamino” is defined as NR^{c}R^{f}, wherein at least one of Re and R is aryl and the other is aryl or hydrogen.

The term “halogen” or “halo,” as used herein, refers to bromine, chlorine, fluorine, or iodine. In some embodiments, the halogen is fluorine. In other embodiments, the halogen is chlorine or bromine.

In various embodiments, the liposomal composition and the phospholipid-polymer-aromatic conjugate used in the method may include any values described herein for the liposomal composition and the phospholipid-polymer-aromatic conjugate.

### EXAMPLES

Certain embodiments are described below in the form of examples. It is impossible to depict every potential application of the invention. Thus, while the embodiments are described in considerable detail, it is not the intention to restrict or in any way limit the scope of the appended claims to such detail, or to any particular embodiment.

General: All reagents were obtained from Sigma-Aldrich (St. Louis, Mo.) and used without further purification. Proton nuclear magnetic resonances (H NMR) spectra were recorded at 600 MHz on a Bruker 600 NMR spectrometer (Bruker, Billerica, Mass.). Carbon nuclear magnetic resonances (¹³C NMR) spectra were recorded at 150 MHz on a Bruker 600 NMR spectrometer. Chemical shifts are reported in parts per million (ppm) from an internal standard acetone (2.05 ppm), chloroform (7.26 ppm), or dimethylsulfoxide (2.50 ppm) for ¹H NMR; and from an internal standard of either residual acetone (206.26 ppm), chloroform (77.00 ppm), or dimethylsulfoxide (39.52 ppm) for ¹³C NMR. NMR peak multiplicities are denoted as follows: s (singlet), d (doublet), t (triplet), q (quartet), bs (broad singlet), dd (doublet of doublet), tt (triplet of triplet), ddd (doublet of doublet of doublet), and m (multiplet). Coupling constants (J) are given in hertz (Hz). High resolution mass spectra (HRMS) were obtained from The Ohio State University Mass Spectrometry and Proteomics Facility, Columbus Ohio; HRMS and matrix-assisted laser desorption/ionization (MALDI) spectra were also obtained from Mass Spectrometry Unit of the BioScience Research Collaborative at Rice University, Houston, Tex. Thin layer chromatography (TLC) was performed on silica gel 60 F254 plates (EMD Chemical Inc., Gibbstown, N.J.) and components were visualized by ultraviolet light (254 nm) and/or phosphomolybdic acid, 20 wt % solution in ethanol. SiliFlash silica gel (230-400 mesh) was used for all column chromatography.

The following methods may be used or adapted to synthesize the conjugates represented by Structural Formula II and compounds represented by Structural Formula III.

### Example 1: Preparation of Conjugate a Using 3+2 “Click” Chemistry

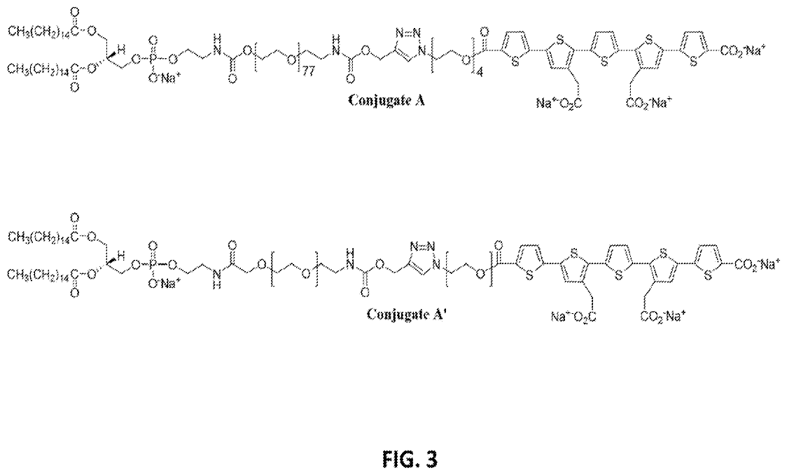

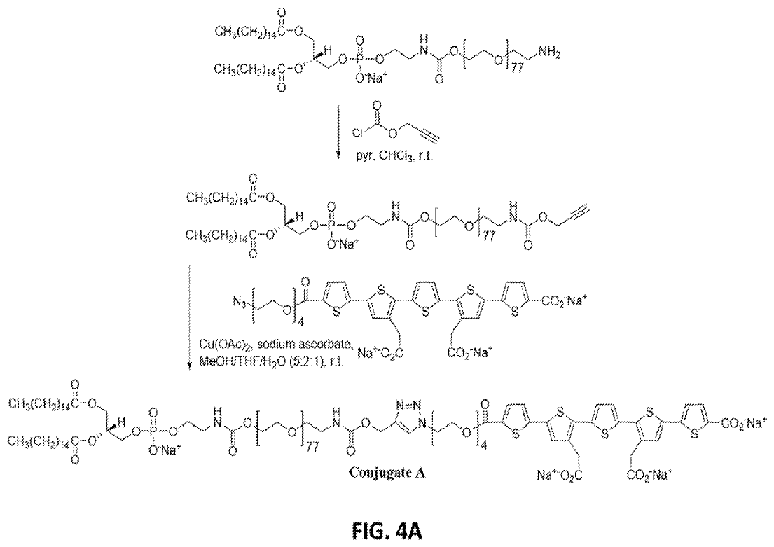

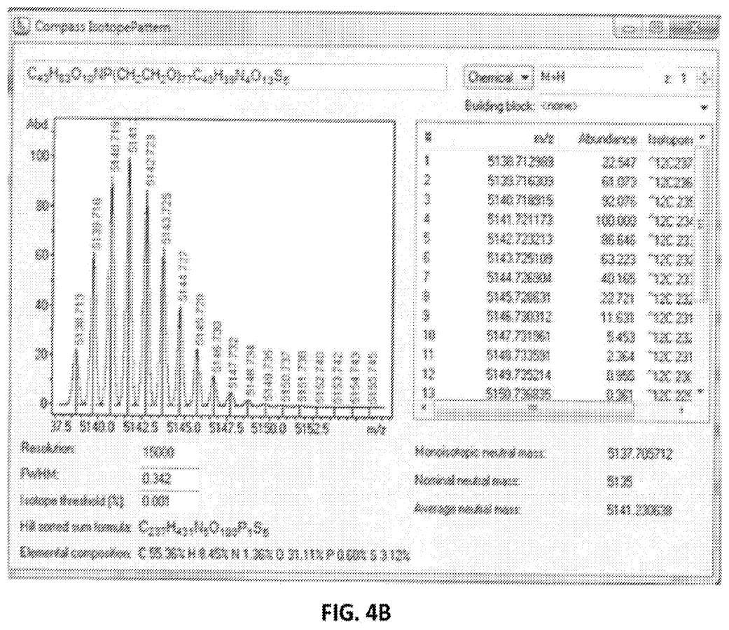

The reactions of Example 1 were conducted according to the scheme shown in FIG. 4A. To a solution of DSPE-PEG_{34K}-NH₂ (1.0 g, 0.24 mmol), pyridine (5 mL, 62.1 mmol), and chloroform (5 mL) was added propargyl chloroformate (50 μL, 0.51 mmol). The resulting mixture was allowed to stir at ambient temperature overnight. The chloroform was removed under reduced pressure and the resulting residue was diluted with a 1:4 EtOH:H₂O solution (20 mL). The solution containing the crude carbamate was loaded into a 2000 MWCO dialysis bag and dialyzed against MES buffer (50 mM, 5 L) for 12 h and twice against water (5 L) for 12 h each. The solution was freeze-dried and the product DSPE-PEG-alkyne (1.08 g, quant.) was obtained as a grey powder, the molecular weight of which was confirmed by MALDI.

To a solution of the DSPE-PEG-alkyne (143.7 mg, 0.034 mmol) and azide-tetraethylene glycol-functionalized pentameric formyl thiophene acetic acid, (“N₃-p-FTAA,” 40 mg, 0.049 mmol) in methanol (3 mL), ethyl acetate (1 mL) and water (1 mL), were added sodium ascorbate (1.07 mg, 0.005 mmol) and copper(II)acetate (0.49 mg, 0.002 mmol). The resulting mixture was stirred at room temperature overnight. The organic solvents were removed in vacuo and the resulting residue diluted with 20% ethanol/water mixture (20 mL). The diluted residue was then loaded in a 2000 MWCO dialysis bag and dialyzed against MES buffer (50 mM, 5 litters) and then water (2×5 liters), for 12 hours each. The water was then removed, by freeze drying to obtain Conjugate A as a white powder (148 mg).

### Example 2A: Preparation of Conjugate a Liposomes

1,2-dihexadecanoyl-sn-glycero-3-phosphocholine (DPPC), Cholesterol (CHOL), Conjugate A, DSPE-DOTA-Gd, and 1,2-distearoyl-sn-glycero-3-phosphoethanolamine-N-[methoxy(polyethylene glycol)-2000] (DSPE-mPEG-2000), in the molar proportions 31:85:40:0.5:25:2.5, respectively, were dissolved in tert-butanol (1 mL), and histidine (10 mM)/saline (150 mM) buffer (9 mL, pH 7.5) was then added. The colloid was hydrated at 60° C. for 45 minutes, and further extruded in a 10 mL Lipex extruder using a 400 nm (10 passes), then followed by 200 nm (10 passes) Nuclepore Track-Etch Membranes. The extruded mixture was then diafiltered using a MicroKros cross-flow diafiltration cartridge (500kD, 20 cm² surface area), using histidine/saline buffer (pH 7.5) and collecting 10 mL fractions (16-time collections). The final liposomes (50 mM, 10 mL) were characterized by dynamic light scattering (DLS) and ICP-AES analysis, and then stored at 4° C.

### Example 2B: Synthesis of Aβ Fibrils

Aβ fibrils were synthesized according to the method of Klunk et al. Ann Neurol, 2004; 55: 306-19, the entire teachings of which are incorporated herein by reference. Briefly, Aβ₍₁₋₄₀₎ peptide (rPeptide, Bogart, GA) was dissolved in phospho-buffered saline, pH 7.4 to a final concentration of 433 μg/mL (100 μM). The solution was stirred using a magnetic stir bar at 700 rpm for 4 h at room temperature to drive the formation of fibrils. The stock solution was aliquoted and stored at −80° C. for future use. The stock solutions were stirred thoroughly before removing aliquots for binding assays to maintain a homogenous suspension of fibrils. The stock solutions were stirred thoroughly prior to removing aliquots for binding assays, to insure a homogenous suspension of fibrils.

### Example 2C: Binding of pFTAA, Conjugate A-Liposomes to Aβ₍₁₋₄₀₎

Conjugate A-liposomes (50 mM, 1 mL) prepared as described above were centrifuged at 14,700 RPM for 10 minutes and at room temperature, and the concentration of free ligand (p-FTAA) in the supernatant was determined by fluorescence (Ex-360 nm, Em-535 nm). Aβ₍₁₋₄₀₎ fibrils in PBS (20 μM, pH 7.5) were incubated with free ligand or Conjugate-A-liposomes at different concentrations and in a reaction volume of 0.3 mL. The set-up was gently agitated at room temperature for 2.5 hours. The fibrils were washed (×3) with 0.3 mL PBS each wash and the supernatant collected at 14, 700 RPM after 2 minutes. The fluorescence for ligands bound to fibrils was obtained from the unbound ligands (supernatant) at excitation and emission wavelengths mentioned above. The binding constant (k_{b}) was determined by plotting the fraction coverage of free ligand or Conjugate-A-liposomes on fibrils against incubation concentration.

### Examples 2A-2C: Discussion

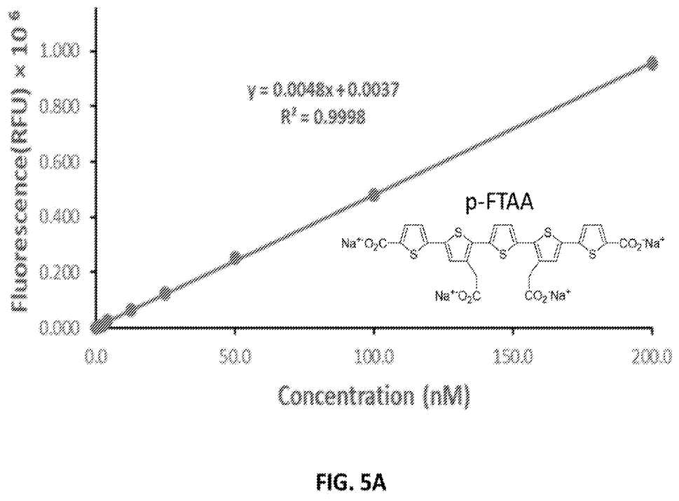

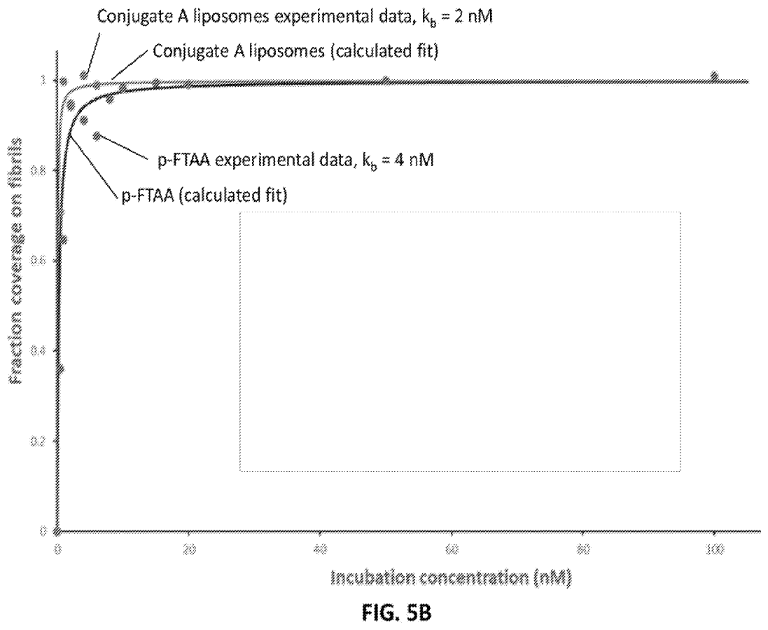

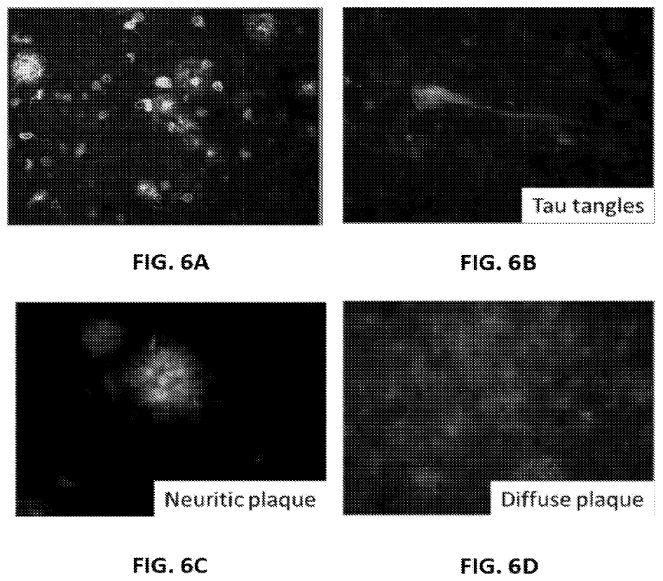

FIG. 4B is a mass spectrum showing that the found average neutral mass for Conjugate-A was 5141.23, calculated for molecular weight 5142.21 (C₂₃₇H₄₃₁N₅O₁₀₀PS₅). The concentrations of phosphorus (25.43 mM) and gadolinium (10.38 mM) in the 50 mM batch of Conjugate-A-liposomes prepared were determined by ICP-AES analysis. Free ligand p-FTAA and Conjugate-A-liposomes were quantified (>43% in the supernatant) used for binding curve assay, using a standard curve (FIG. 5A). The supernatant from centrifuged Conjugate-A-liposomes was used in binding experiments, but not the intact Conjugate-A-liposomes, to get rid of aggregates which can impact binding efficiency. Surprisingly and unexpectedly, the binding constant (k_{b}) for Conjugate-A-liposomes was 2.0 nM, half of that for the free ligand, p-FTAA, which was 4 nM. (FIG. 5B) Moreover, despite the much larger size compared to the free ligand, the Conjugate-A-liposomes readily entered deep into brain tissues to stain concentrated Aβ deposits (FIG. 6A), tau tangles (FIG. 6B), neuritic plaques (FIG. 6C), and diffuse plaques (FIG. 6D).

### Example 3A: Preparation of Conjugate a Liposomes

Liposomes of 50 mM lipid content were prepared by dissolving 1,2-dihexadecanoyl-sn-glycero-3-phosphocholine (DPPC), Cholesterol (CHOL), 1,2-distearoyl-sn-glycero-3-phosphoethanolamine-N-[methoxy(polyethylene glycol)-2000] (DSPE-mPEG-2000) and Conjugate A, in the molar proportions 31:85:40:0.5:25:2.5, respectively, in ethanol (1 mL). The ethanolic colloid was hydrated at 62° C. for 45 minutes with histidine saline (10 mM)/saline (150 mM) buffer (9 mL, pH 7.5) to form liposomes. The liposomes were extruded in a 10 mL Lipex extruder using a 400 nm (3 passes), then followed by 100 nm (4 passes) Nuclepore Track-Etch Membranes. The extruded mixture was then diafiltered overnight using a MicroKros cross-flow diafiltration cartridge (500kD, 20 cm² surface area), using histidine/saline buffer (pH 7.5) to remove ethanol. The final liposomes (50 mM, 10 mL) were characterized by dynamic light scattering (DLS) and ICP-AES analysis, and then stored at 4° C.

### Example 3B: Binding Studies with α-Synuclein Fibrils

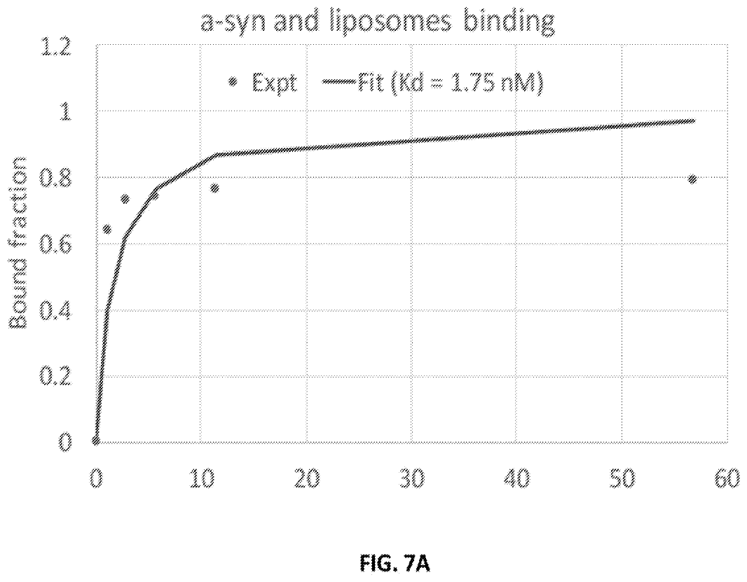

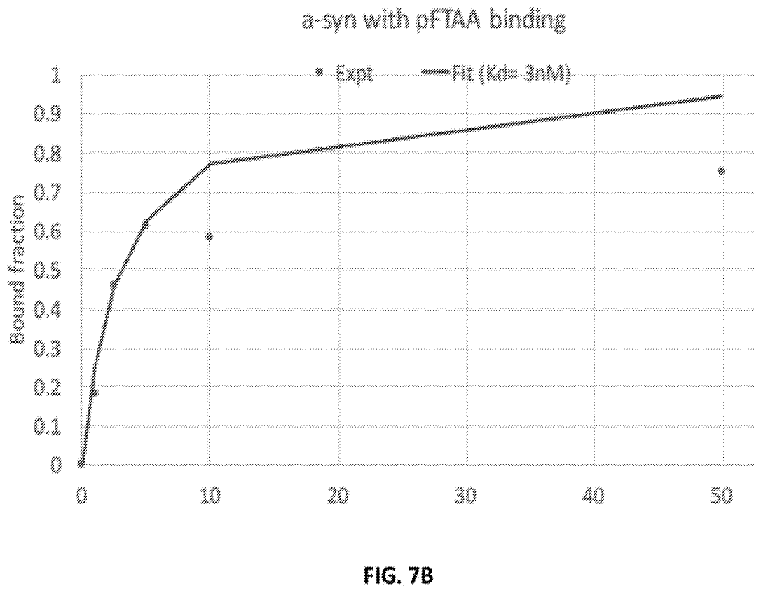

Different dilutions of Conjugate A liposomes or p-FTAA were added and mixed with 5 μM of α-Synuclein fibrils and incubated for 2 hours. The mixture was centrifuged at 21000 g for 5 mins. The supernatant was removed and assayed for fluorescence of p-FTAA that was unbound to the fibrils. Excitation of 405 nm and emission of 574 nm emission was used. A standard curve from known concentrations of liposomes or pFTAA was made. The bound fraction was calculated as the difference between the total pFTAA or pFTAA-liposomes that was incubated and the unbound fraction. A dissociation constant, K_{d} of 1.75 nM was determined for Conjugate A liposomes (FIG. 7A). A dissociation constant of 3 nM was determined for the p-FTAA molecule (FIG. 7B).

### Example 4A: Formation of Tau Fibrils

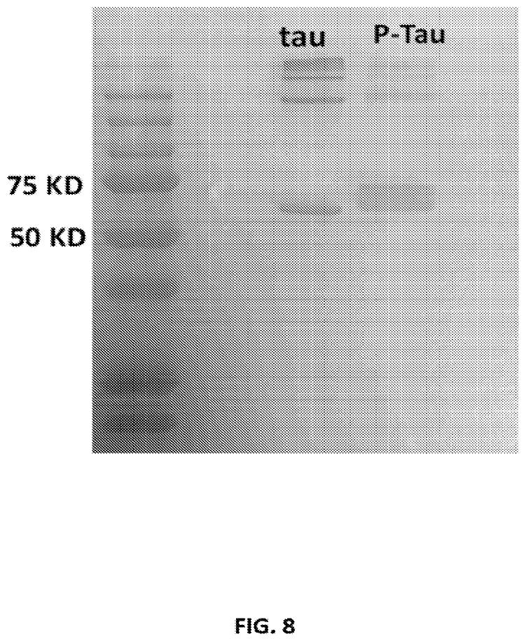

Lyophilized tau-441 (2N₄R) was dissolved in buffer containing 40 mM HEPES, 5 mM EGTA, 3 mM MgCl2, pH 7.5. Tau was phosphorylated with GSK-3b in the presence of 2 mM ATP at 30° C. for 40 hours. Tau was used at 30-75 μM concentration, and GSK-3b was used at 0.02-0.08 U/pmol of tau. SDS PAGE gel was run to confirm the phosphorylation of tau (FIG. 8).

To make fibrils, the phosphorylated tau was reacted with arachidonic acid (ARA). The p-tau was diluted in 10 mM HEPES, 1 mM EDTA, 5 mM DTT and 150 mM NaCl at pH˜7.6 to final concentration of 32 μM and ARA at 37 times molar excess of tau was added. The mixture was incubated for 2 days at 37° C. for 2 days. Fresh DTT was supplemented daily. The oligomers formed were used as seeds for fibril formation. 8 μM of tau seeds were used to form fibrils with 30 μM p-tau in 10 mM HEPES, 1 mM EDTA, 5 mM DTT and 150 mM NaCl at pH˜7.6. ARA was used at 37 times molar excess over p-tau monomer and incubated at 37° C. for 2 days. Fresh DTT was added every day.

Fibril formation was monitored by monitoring Thioflavin T fluorescence. Thioflavin T is non-fluorescent molecule, but in the presence of tau aggregates, it binds to tau fibrils and exhibits fluorescence with excitation of 405 nm and emission of 535 nm.

### Example 4B: Binding Studies with Tau Fibrils

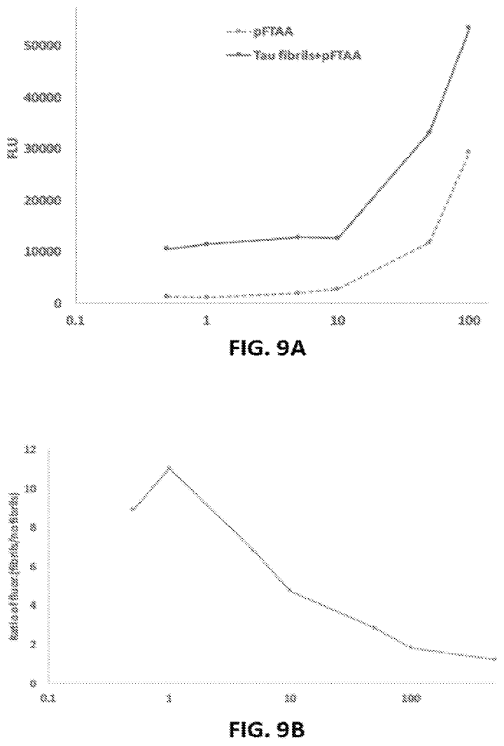

Different dilutions of p-FTAA molecule were added and mixed with 0.85 μM of tau fibrils and incubated for 2 hours. The mixture was assayed as is without separation of bound and unbound fibrils. Excitation at 360 nm and emission at 535 nm was used for detection of p-FTAA binding to tau fibrils. The p-FTAA mix with tau fibrils showed increase in fluorescence in comparison to p-FTAA only fluorescence (FIG. 9A) suggesting binding of pFTAA to tau fibrils. The ratio of the fluorescence of fibril—pFTAA mix to pFTAA only is shown in FIG. 9B.

As stated above, while the present application has been illustrated by the description of embodiments, and while the embodiments have been described in considerable detail, it is not the intention to restrict or in any way limit the scope of the appended claims to such detail. Additional advantages and modifications will readily appear to those skilled in the art, having the benefit of this application. Therefore, the application, in its broader aspects, is not limited to the specific details and illustrative examples shown. Departures may be made from such details and examples without departing from the spirit or scope of the general inventive concept.

1-33. (canceled)

34. A liposomal composition, comprising:
a first phospholipid;
a sterically bulky excipient that is capable of stabilizing the liposomal composition;
a second phospholipid that is derivatized with a first polymer;
a macrocyclic gadolinium-based imaging agent; and
a third phospholipid that is derivatized with a second polymer, the second polymer being conjugated to a targeting ligand, the targeting ligand being represented by: 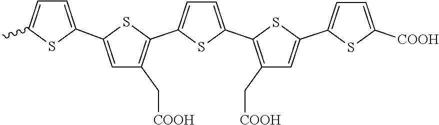
or a salt thereof, and
wherein the third phospholipid that is derivatized with a second polymer comprises: 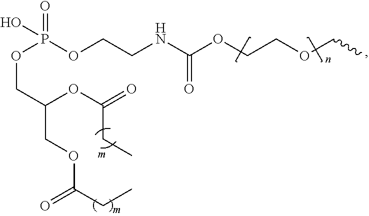
or a salt thereof, wherein the variable n is any integer from about 70 to about 90, and wherein the variable m is one of: 12, 13, 14, 15, 16, 17, or 18.

35. The liposomal composition of claim 34, wherein the sterically bulky excipient that is capable of stabilizing the liposomal composition comprises cholesterol (“Chol”).

36. The liposomal composition of claim 34, wherein the second phospholipid that is derivatized with a first polymer comprises 1,2-distearoyl-sn-glycero-3-phosphoethanolamine-N-(methoxy (polyethylene glycol)-2000) (“DSPE-mPEG2000”).

37. The liposomal composition of claim 34, wherein the macrocyclic gadolinium-based imaging agent comprises:

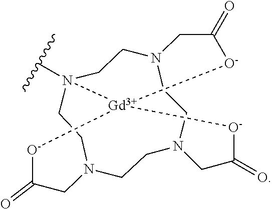

38. The liposomal composition of claim 37, wherein the macrocyclic gadolinium-based imaging agent is conjugated to a fourth phospholipid to comprise: 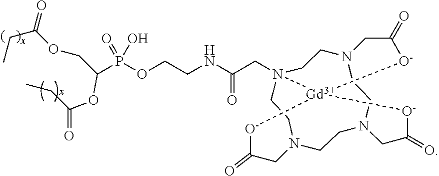
or a salt thereof, and wherein the variable x is one of: 12, 13, 14, 15, 16, 17, or 18.

39. The liposomal composition of claim 38, wherein the variable x is 16 (the conjugate: “Gd(III)-DOTA-DSPE”).

40. The liposomal composition of claim 34, characterized in that the liposomal composition exhibits a binding constant (K_{b}) with respect to amyloid beta fibrils that is less than the K_{b} with respect to amyloid beta fibrils exhibited by the free targeting ligand.

41. The liposomal composition of claim 34, characterized in that the liposomal composition exhibits a dissociation constant (K_{d}) with respect to α-synuclein fibrils that is less than the K_{d} with respect to α-synuclein fibrils exhibited by the free targeting ligand.

42. A liposomal composition, comprising:
a macrocyclic gadolinium-based imaging agent conjugated to a first phospholipid comprising: 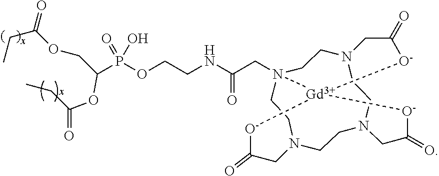
or a salt thereof, and wherein the variable x is one of 12, 13, 14, 15, 16, 17, or 18; and
a second phospholipid that is derivatized with a polymer, the polymer being conjugated to a targeting ligand, the conjugate of the second phospholipid, the polymer, and the targeting ligand comprising: 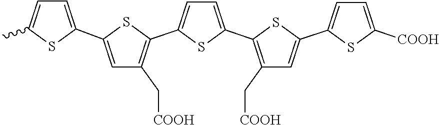
or a salt thereof, and 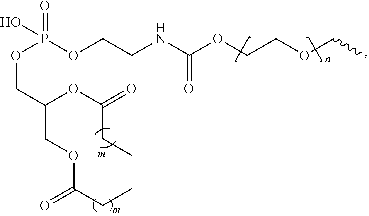
or a salt thereof, wherein the variable n is any integer from about 70 to about 90, and wherein the variable m is one of: 12, 13, 14, 15, 16, 17, or 18.

43. The liposomal composition of claim 42, characterized in that the liposomal composition exhibits a binding constant (K_{b}) with respect to amyloid beta fibrils that is less than the K_{b} with respect to amyloid beta fibrils exhibited by the free targeting ligand.

44. The liposomal composition of claim 42, characterized in that the liposomal composition exhibits a dissociation constant (K_{d}) with respect to α-synuclein fibrils that is less than the K_{d} with respect to α-synuclein fibrils exhibited by the free targeting ligand.

45. A liposomal composition, comprising:
DPPC;
Chol;
DSPE-mPEG2000;
Gd(III)-DOTA-DSPE; and
a phospholipid that is derivatized with a polymer, the polymer being conjugated to a targeting ligand, the conjugate of the phospholipid, the polymer, and the targeting ligand comprising: 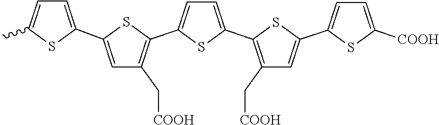
or a salt thereof, and 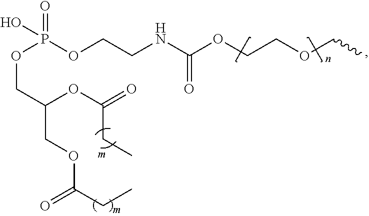
or a salt thereof, wherein the variable n is any integer from about 70 to about 90, and wherein the variable m is one of: 12, 13, 14, 15, 16, 17, or 18.

46. The liposomal composition of claim 45, wherein the molar proportions of the components are DPPC: about 32; cholesterol: about 40; the conjugate: about 0.5; Gd(III)-DOTA-DSPE: about 25; and DSPE-mPEG2000: about 2.5.

47. The liposomal composition of claim 45, characterized in that the liposomal composition exhibits a binding constant (K_{b}) with respect to amyloid beta fibrils that is less than the K_{b} with respect to amyloid beta fibrils exhibited by the free targeting ligand.

48. The liposomal composition of claim 45, characterized in that the liposomal composition exhibits a dissociation constant (K_{d}) with respect to α-synuclein fibrils that is less than the K_{d} with respect to α-synuclein fibrils exhibited by the free targeting ligand.
